(19) United States
(12) Patent Application Publication
(10) Pub. No.: US 2026/0325587 A1
(43) Pub. Date: Oct. 22, 2026

(54) COMPOSITIONS FOR AND METHODS OF MODULATING THE GUT MICROBIOME

(71) Applicants: The George Washington University, Washington, DC (US); The United States Government as represented by the Department of Veterans Affairs, Washington, DC (US)

(72) Inventors: Cindy Liu, Washington, DC (US); Daniel Park, Washington, DC (US); Lance B. Price, Washington, DC (US); James R. Johnson, Washington, DC (US)

(21) Appl. No.: 19/654,028

(22) Filed: Apr. 21, 2026

Related U.S. Application Data
(63) Continuation of application No. PCT/US2024/054449, filed on Nov. 4, 2024.
(60) Provisional application No. 63/547,302, filed on Nov. 3, 2023.

(51) Int. Cl. A61K 35/74; A61P 31/04; G01N 33/50

(52) U.S. Cl. CPC A61K 35/74; A61P 31/04; G01N 33/502; G01N 2800/52

(57) ABSTRACT

Disclosed herein are methods of reducing the risk of developing a drug-resistant E. coli infection and methods of treating and/or preventing drug-resistant E. coli colonization and/or infection.

### I. CROSS-REFERENCE TO RELATED APPLICATIONS

This application is a continuation of International Patent Application No. PCT/US2024/054449, filed on Nov. 4, 2024, which claims priority to U.S. Provisional Application No. 63/547,302 filed on Nov. 3, 2023, the entire disclosures of which are hereby incorporated by reference, for all purposes, as if fully set forth herein.

### II. STATEMENT REGARDING FEDERALLY SPONSORED RESEARCH OR DEVELOPMENT

This invention was made with government support under AI117654 awarded by the National Institute of Allergy and Infectious Diseases, AI104681 awarded by the National Institute of Health, and CX000920 awarded by the Department of Veterans Affairs. The government has certain rights in the invention.

### III. BACKGROUND

Escherichia coli (E. coli) sequence type 131 (ST131), especially its recently emerged H30R subclone and the two main clonal subsets within H30R (H30R1 and H30Rx) cause a substantial portion of anti-microbial resistant infections in the U.S. and worldwide. (Johnson J R, et al. (2017) Open Forum Infect Dis. 4: ofx089). Both H30R1 and H30Rx are characterized by fluoroquinolone resistance. Additionally, many H30R strains-especially within H30Rx—are co-resistant to multiple other antimicrobial agents, most notably-advanced beta-lactams due to plasmidic or chromosomal extended-spectrum beta-lactamases and, increasingly, carbapenemases.

A key feature of ST131-H30R (including the H30R1 and H30Rx subsets) that underlies its pandemic emergence is its ability to stably colonize the human gastrointestinal tract, (Tchesnokova V L, et al. (2020) Clin Infect Dis. 70:937-939; Johnson J R, et al. (2022) J Infect Dis. 225:2197-2207). This is mediated, at least in part, through bacterial factors such as FimH, (Sokurenko E V, et al. (1998) Proc Natl Acad Sci USA. 95:8922-8926; Connell I, et al. (1996) Proc Natl Acad Sci USA. 93:9827-9832) fluoroquinolone resistance, (Johnson J R, et al. (2012) Antimicrob Agents Chemother. 56:2364-2370) and accessory traits such as iron uptake systems and protectins. (Johnson J R, et al. (2022) J Infect Dis. 225:2197-2207). Thus, gut colonization by ST131-H30R significantly increases the risk for subsequent infection. (Tchesnokova V L, et al. (2020) Clin Infect Dis. 70:937-939). Individuals can become colonized by antibiotic-resistant E. coli via horizontal transmission, such as through exposures from international travel (Langelier C, et al. (2019) Emerg Infect Dis. 25:1380-1383) within households, (Valverde A, et al. (2008) J Clin Microbiol. 46:2796-2799) or in the healthcare setting. (Hilty M, et al. (2012) Clin Infect Dis. 55:967-975).

There is thus an urgent need to develop approaches to decreasing transmission and infections by ST131 E. coli, especially H30R1 and H30Rx.

### IV. BRIEF DESCRIPTION OF THE FIGURES

FIG. 1 shows the participant flow for inclusion into cross-sectional and longitudinal analyses, for the ST131, H30, and H30R categories. Flowchart for the cross-sectional and longitudinal data, including characterization of the number of families, individuals, samples, and molecular characterization of E. coli isolates. High-level associations between taxa and E. coli ST131 carriage are provided at the bottom, with cross-sectional association findings being validated in a subset of individuals with available longitudinal data.

FIG. 2A-FIG. 2B show the gut microbiota genera proportional abundance among exposed, sustained H30-negative (FIG. 2A) and sustained H30R loss participants (FIG. 2B). Each group of adjoined columns represents a sequential (from left to right) time series of samples from an individual participant. Columns represent the proportional abundance of genera at each time point. Colored horizontal bars above the columns represent H30R (red) and ST131 (non-H30R, orange) status over time for each participant. Arrows represent the time point where H30R is no longer detected for participants with sustained loss, which is generally accompanied by loss of Collinsella.

FIG. 3 shows phylum-level proportional abundance of gut microbiome taxa among participants at baseline, ordered by Actinobacteria proportional abundance and stratified by ST131 and Escherichia/Shigella status.

FIG. 4 shows a genus-level proportional abundance of gut microbiome taxa among participants at baseline, ordered by Collinsella proportional abundance and stratified by ST131 and Escherichia/Shigella status.

FIG. 5A-FIG. 5B show a phylum-level proportional abundance of gut microbiome taxa among participants at baseline, stratified by ST131 carriage status.

FIG. 6A-FIG. 6B show a phylum-level alpha diversity (Shannon index) at baseline, stratified by carriage status of (FIG. 6A) ST131-negative and ST131-H30R subclones, and (FIG. 6B) ST131-negative and ST131-positive sequence types.

### V. BRIEF SUMMARY

Disclosed herein is a composition, comprising: one or more bacteria belonging to the Alistipes genus. Disclosed herein is a composition, comprising: one or more bacteria belonging to the Akkermansia genus. Disclosed herein is a composition, comprising: one or more bacteria belonging to the Alistipes genus and one or more bacteria belonging to the Akkermansia genus.

Disclosed herein is a composition, comprising: one or more bacteria belonging to the Alistipes genus and one or more probiotics. Disclosed herein is a composition, comprising: one or more bacteria belonging to the Akkermansia genus and one or more probiotics. Disclosed herein is a composition, comprising: one or more bacteria belonging to the Alistipes genus and one or more bacteria belonging to the Akkermansia genus and one or more probiotics. Disclosed herein is a composition, comprising: one or more bacteria belonging to the Alistipes genus and one or more prebiotics. Disclosed herein is a composition, comprising: one or more bacteria belonging to the Akkermansia genus and one or more prebiotics. Disclosed herein is a composition, comprising: one or more bacteria belonging to the Alistipes genus and one or more bacteria belonging to the Akkermansia genus and one or more prebiotics.

Disclosed herein is a composition, comprising: one or more agents that can reduce the intestinal carriage associated with the colonization of drug-resistant E. coli bacteria, thereby reducing the risk of an infection. Disclosed herein is a composition, comprising: one or more agents that can reduce the proportional abundance of one or more bacteria associated with the carriage and/or persistence of drug-resistant E. coli bacteria, thereby reducing the degree and/or severity of colonization by a drug-resistance E. coli.

Disclosed herein is a composition, comprising: one or more agents that can inhibit proliferation of Asaccharobacter, Bifidobacterium, Collinsella, Eggerthella, Gardnerella, Gordonibacter, or any combination thereof in the gut microbiome of the subject.

Disclosed herein is a composition, comprising: one or more agents that can increase proliferation of Alistipes and/or Akkermansia.

Disclosed herein is a method of reducing the risk of developing a drug-resistant E. coli infection, the method comprising administering to a subject at risk of developing an E. coli infection a therapeutically effective amount of a composition comprising one or more bacteria belonging to the Alistipes genus and/or Akkermansia genus, wherein following the administering step the composition reduces the intestinal carriage associated with the colonization of drug-resistant E. coli bacteria, thereby reducing the risk of infection. Disclosed herein is a method of reducing the risk of developing a drug-resistant E. coli infection, the method comprising administering to a subject at risk of developing a drug-resistant E. coli infection a therapeutically effective amount of a composition that modulates the intestinal carriage associated with the colonization of drug-resistant E. coli bacteria, thereby reducing the risk of a drug-resistant E. coli infection.

Disclosed herein is a method of reducing the degree and/or severity of a drug-resistant E. coli infection in a subject, the method comprising administering to a subject in need thereof a therapeutically effective amount of a composition comprising one or more bacteria belonging to the Alistipes genus and/or Akkermansia genus, wherein following the administering step the composition reduces the intestinal carriage and/or persistence of one or more E. coli bacteria, thereby reducing the degree and/or severity of an infection of a drug-resistant E. coli. Disclosed herein is a method of reducing the degree and/or severity of a drug-resistant E. coli colonization in a subject, the method comprising administering to a subject in need thereof a therapeutically effective amount of a composition comprising one or more bacteria belonging to the Alistipes genus and/or Akkermansia genus, wherein following the administering step the composition reduces the intestinal carriage and/or persistence of one or more E. coli bacteria, thereby reducing the degree and/or severity of colonization by a drug-resistance E. coli.

Disclosed herein is a method of reducing the degree and/or severity of a drug-resistant E. coli colonization in a subject, the method comprising administering to a subject in need thereof a therapeutically effective amount of a composition that modulates the intestinal carriage associated with the colonization of drug-resistant E. coli bacteria, wherein, following the administering step, the composition reduces the proportional abundance of one or more bacteria associated with the carriage and/or persistence of drug-resistant E. coli bacteria, thereby reducing the degree and/or severity of colonization by a drug-resistance E. coli. Disclosed herein is a method of increasing resistance to a drug-resistant E. coli infection and/or colonization in a subject, the method comprising administering to a subject in need thereof a therapeutically effective amount of a composition comprising one or more bacteria belonging to the Alistipes genus and/or Akkermansia genus, wherein following the administering step the composition reduces the intestinal carriage and/or persistence of one or more E. coli bacteria, thereby increasing the subject's resistance to an infection and/or colonization of a drug-resistance E. coli.

Disclosed herein is a method of increasing resistance to a drug-resistant E. coli infection and/or colonization in a subject, the method comprising administering to a subject in need thereof a therapeutically effective amount of a composition comprising one or more bacteria belonging to the Alistipes genus and/or Akkermansia genus, wherein following the administering step the composition reduces the intestinal carriage and/or persistence of one or more E. coli bacteria, thereby increasing the subject's resistance to an infection and/or colonization of a drug-resistance E. coli.

### VI. DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. DEFINITIONS

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

In an aspect, when referring to any numerical value, the term “about” means a value falling within a range that is +10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

In an aspect, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

In an aspect, the term “subject” refers to the target of administration, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have a respiratory infection, be suspected of having a respiratory infection, or be at risk of developing a respiratory infection.

In an aspect, the term “diagnosed” means having been subjected to an examination by a person of skill, for example, a physician, and found to have a condition that can be diagnosed or treated by one or more of the disclosed biotherapeutics, the disclosed pharmaceutical formulations, or a combination thereof, or by one or more of the disclosed methods. For example, “diagnosed with an infection” means having been subjected to an examination by a person of skill, for example, a physician, and found to have a condition (such as, for example, a respiratory infection) that can be treated by one or more of the disclosed biotherapeutics, the disclosed pharmaceutical formulations, or any combination thereof, or by one or more of the disclosed methods. For example, “suspected of having an infection” can mean having been subjected to an examination by a person of skill, for example, a physician, and found to have a condition (such as a respiratory infection) that can likely be treated by one or more of by one or more of the disclosed biotherapeutics, the disclosed pharmaceutical formulations, or any combination thereof, or by one or more of the disclosed methods. In an aspect, an examination can be physical, can involve various tests (e.g., blood tests, genotyping, biopsies, etc.), scans (e.g., CT scans, PET scans, etc.), and assays (e.g., enzymatic assay), or a combination thereof.

A “patient” refers to a subject at risk of or afflicted with an infection such as, for example, a respiratory infection. In an aspect, a patient can refer to a subject that has been diagnosed with or is suspected of having a disease or disorder such as a respiratory infection. In an aspect, a patient can refer to a subject that has been diagnosed with or is suspected of having a respiratory infection and is seeking treatment or receiving treatment for the respiratory infection). In an aspect, a patient can be a healthy subject not having an infection and not at risk of developing an infection.

In an aspect, “probiotic” refers to a microorganism such as a bacteria or yeast that is associated with positive effect for and/or confers an advantage to a subject. In an aspect, a probiotic can comprise a single bacterial strain, a single bacterial strain (such as, for example, a strain of the bacterial species Alistipes or Akkermansia), a consortium of bacterial strains from the same genus or species, or a consortium of bacterial strains of different genera or species (e.g., Alistipes and Akkermansia). “Probiotic” as used herein refers to factors secreted from a microorganism such as a bacteria or yeast that are associated with positive effect for and/or confer an advantage to a subject. In an aspect, a probiotic can comprise factors secreted from a single bacterial strain, a single bacterial species (such as, for example, Alistipes or Akkermansia), a consortium of bacterial strains from the same genus or species, or a consortium of bacterial strains from different genera or species (e.g., Alistipes and Akkermansia.)

In an aspect, the term “postbiotics” refers to functional bioactive compounds produced by probiotics and can be used to promote health. The term postbiotics can be considered as a generic term for all synonyms and related terms for these microbial components. Therefore, postbiotics include metabolites, short chain fatty acids (SCFAs such as acetic acid, propionic acid, and butyric acid, etc.), microbial cell fractions, functional proteins, extracellular polysaccharides (EPS), cell lysates, acids, phenyl-lactic acid, volatile organic compounds (VOCs), B-vitamin synthesis (biotin, cobalamine, folic acid, nicotinic acid, pantothenic acid, pyridoxin, riboflavin, and thiamine), muropeptides derived from peptide glycans, antibacterial peptides (AMP) and pib types.

In an aspect, the phrase “identified to be in need of treatment for a respiratory infection,” or the like, refers to selection of a subject based upon need for treatment of the respiratory infection or a subject at risk of developing a respiratory infection. For example, a subject can be identified as having a need for treatment of a respiratory infection based upon an earlier diagnosis by a person of skill and thereafter subjected to treatment for the respiratory infection. In an aspect, the identification can be performed by a person different from the person making the diagnosis. In an aspect, the administration can be performed by one who performed the diagnosis.

In an aspect, “inhibit,” “inhibiting”, and “inhibition” mean to diminish or decrease an activity, level, response, condition, severity, disease, or other biological parameter. This can include, but is not limited to, the complete ablation of the activity, growth, colonization, endocytosis, spread, expression level, response, condition, severity, disease, or other biological parameter. This can also include, for example, a 10% inhibition or reduction in the activity, growth, colonization, endocytosis, spread, expression level, response, condition, severity, disease, or other biological parameter as compared to the native or control level (e.g., a subject not receiving one or more of the disclosed biotherapeutics, the disclosed pharmaceutical formulations, or a combination thereof). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to a native or control level (e.g., a subject not receiving one or more of the disclosed biotherapeutics, the disclosed pharmaceutical formulations, or a combination thereof). In an aspect, the inhibition or reduction can be 0-25%, 25-50%, 50-75%, or 75-100% as compared to native or control levels. In an aspect, a native or control level can be a pre-infection level (such as a pre-respiratory infection state).

The words “treat” or “treating” or “treatment” include palliative treatment, that is, treatment designed for the relief of symptoms rather than the curing of the infection, disease, pathological condition, or disorder; preventative treatment, that is, treatment directed to minimizing or partially or completely inhibiting the development of the associated infection, disease, pathological condition, or disorder; and supportive treatment, that is, treatment employed to supplement another specific therapy directed toward the improvement of the associated infection, disease, pathological condition, or disorder. In an aspect, the terms cover any treatment of a subject, including a mammal (e.g., a human), and includes: (i) preventing the undesired infection, physiological change, disease, pathological condition, or disorder from occurring in a subject that can be predisposed to the disease but has not yet been diagnosed as having it; (ii) inhibiting the infection, physiological change, disease, pathological condition, or disorder, i.e., arresting its development; or (iii) relieving the infection, physiological change, disease, pathological condition, or disorder, i.e., causing regression of the disease. For example, in an aspect, treating an infection can reduce the severity of an established infection in a subject by 1%-100% as compared to a control (such as, for example, an individual not having a respiratory infection). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of the infection (e.g., a respiratory infection). For example, treating an infection can reduce one or more symptoms of a disease or disorder in a subject by 1%-100% as compared to a control (such as, for example, an individual not having an infection such as a respiratory infection). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established infection. It is understood that treatment does not necessarily refer to a cure or complete ablation or eradication of an infection. However, in an aspect, treatment can refer to a cure or complete ablation or eradication of an infection (such as a respiratory infection).

In an aspect, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a respiratory infection is intended. The words “prevent”, “preventing”, and “prevention” also refer to prophylactic or preventative measures for protecting or precluding a subject (e.g., an individual) not having an infection (such as a respiratory infection) or related complication from progressing to that complication. In an aspect, preventing a respiratory infection is intended.

In an aspect, the terms “administering” and “administration” refer to any method of providing to a subject one or more of the disclosed biotherapeutics, the disclosed pharmaceutical formulations, or any combination thereof. Such methods are well known to those skilled in the art and include, but are not limited to, the following: oral administration, transdermal administration, administration by inhalation, nasal administration or administration into other sites in the upper respiratory tract, topical administration, in utero administration, intratumoral administration, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can be continuous or intermittent. Administration can comprise a combination of one or more routes.

In an aspect, the skilled person can determine an efficacious dose, an efficacious schedule, and an efficacious route of administration of one or more of the disclosed biotherapeutics, the disclosed pharmaceutical formulations, or any combination thereof to treat or prevent an infection (such as a respiratory infection). In an aspect, the skilled person can also alter, change, or modify an aspect of an administering step to improve efficacy of the disclosed biotherapeutics, the disclosed pharmaceutical formulations, or any combination thereof.

By “determining the amount” is meant both an absolute quantification of a particular analyte (e.g., biomarker for an infection) or a determination of the relative abundance of a particular pathogenic and/or non-pathogenic bacteria (e.g., ST131 E. coli). The phrase includes both direct or indirect measurements of abundance or both.

In an aspect, “modifying the method” can comprise modifying or changing one or more features or aspects of one or more steps of a disclosed method. In an aspect, a method can be altered by changing the amount of the disclosed biotherapeutic, the disclosed pharmaceutical formulation, or disclosed therapeutic agent administered to a subject, or by changing the frequency of administration of the disclosed biotherapeutic, the disclosed pharmaceutical formulation, or the disclosed therapeutic agent, by changing the duration of time that the disclosed biotherapeutic, the disclosed pharmaceutical formulation, or the disclosed therapeutic agent, or by substituting for one or more of the disclosed components and/or reagents with a similar or equivalent component and/or reagent. The same applies to all disclosed biotherapeutics, the disclosed pharmaceutical formulations, or any combination thereof, or all combinations thereof.

In an aspect, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

In an aspect, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

In an aspect, “concurrently” means (1) simultaneously in time, or (2) at different times during the course of a common treatment schedule.

In an aspect, the term “contacting” refers to bringing one or more of the disclosed biotherapeutics, the disclosed pharmaceutical formulations, or any combination thereof together with a target area or intended target area (e.g., such as an aspect of the respiratory system affected by an infection) in such a manner that the disclosed biotherapeutic, the disclosed pharmaceutical formulation, or any combination thereof can exert an effect on the intended target or targeted area either directly or indirectly. A target area or intended target area can be one or more parts of a subject's respiratory system (e.g., the subject's mouth, nose, ears, sinuses, nasopharynx or oropharynx, trachea, bronchial tubes, lungs, alveoli, bronchioles, capillaries, lung lobes, pleura, cilia, epiglottis, larynx, or any combination thereof). In an aspect, a target area or intended target area can be any cell or any organ infected by E. coli. In an aspect, a target area or intended target area can be any organ, tissue, or cells that are affected by an infection.

In an aspect, “determining” can refer to measuring or ascertaining the presence and severity of an infection, such as a respiratory infection. Methods and techniques used to determine the presence and/or severity of an infection are typically known to the medical arts. For example, the art is familiar with the ways to identify and/or diagnose the presence, severity, or both of an infection (such as, for example, a respiratory infection).

In an aspect, “effective amount” and “amount effective” can refer to an amount that is sufficient to achieve the desired result such as, for example, the treatment and/or prevention of an infection (e.g., a respiratory infection) or a suspected infection. In an aspect, the terms “effective amount” and “amount effective” can refer to an amount that is sufficient to achieve the desired an effect on an undesired condition (e.g., a respiratory infection). For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount of a disclosed biotherapeutic, a disclosed pharmaceutical formulation, or any combination thereof that (i) treats the particular infection, disease, condition, or disorder (e.g., a respiratory infection), (ii) attenuates, ameliorates, or eliminates one or more symptoms of the particular infection, disease, condition, or disorder (e.g., a respiratory infection), or (iii) delays the onset of one or more symptoms of the particular infection, disease, condition, or disorder described herein (e.g., a respiratory infection). The specific therapeutically effective dose level for any particular patient can depend upon a variety of factors including the disorder being treated and the severity of the infection; the disclosed biotherapeutics, the disclosed pharmaceutical formulations, or any combination thereof employed in the disclosed methods; the age, body weight, general health, sex and diet of the patient; the time of administration; the route of administration; the rate of excretion of the disclosed biotherapeutics, the disclosed pharmaceutical formulations, or any combination thereof employed; the duration of the treatment; other drugs used in combination or coincidental with the disclosed biotherapeutics, the disclosed pharmaceutical formulations, or any combination thereof, and other like factors well known in the medical arts. For example, it is well within the skill of the art to start doses of the disclosed biotherapeutics, the disclosed pharmaceutical formulations, or any combination thereof at levels lower than those required to achieve the desired therapeutic effect and to gradually increase the dosage until the desired effect is achieved. If desired, then the effective daily dose can be divided into multiple doses for purposes of administration. Consequently, a single dose of the disclosed biotherapeutics, the disclosed pharmaceutical formulations, or any combination thereof can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of an infection.

“Sequence identity” and “sequence similarity” can be determined by alignment of two peptide or two nucleotide sequences using global or local alignment algorithms. Sequences may then be referred to as “substantially identical” or “essentially similar” when they are optimally aligned. For example, sequence similarity or identity can be determined by searching against databases such as FASTA, BLAST, etc., but hits should be retrieved and aligned pairwise to compare sequence identity. Two proteins or two protein domains, or two nucleic acid sequences can have “substantial sequence identity” if the percentage sequence identity is at least 70%, 75%, 80%, 85%, 90%, 95%, 98%, 99% or more, preferably 90%, 95%, 98%, 99% or more. Such sequences are also referred to as “variants” herein, e.g., other variants of a missing, deficient, and/or mutant protein or enzyme. It should be understood that sequence with substantial sequence identity do not necessarily have the same length and may differ in length. For example, sequences that have the same nucleotide sequence but of which one has additional nucleotides on the 3′- and/or 5′-side are 100% identical.

In an aspect, the term “package insert” is used to refer to instructions customarily included in commercial packages of therapeutic products, that contain information about the indications, usage, dosage, administration, contraindications and/or warnings concerning the use of such therapeutic products.

In an aspect, the term “in combination” in the context of the administration of other therapies (e.g., other agents) includes the use of more than one therapy (e.g., drug therapy). Administration “in combination with” one or more further therapeutic agents includes simultaneous (e.g., concurrent) and consecutive administration in any order. The use of the term “in combination” does not restrict the order in which therapies are administered to a subject. By way of non-limiting example, a first therapy (e.g., a disclosed biotherapeutic or a disclosed pharmaceutical formulation) may be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., a disclosed biotherapeutic or a disclosed pharmaceutical formulation) to a subject having or diagnosed with an infection (such as, for example, a respiratory infection).

Disclosed are the components to be used to prepare the disclosed biotherapeutics and the disclosed pharmaceutical formulations used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### B. Compositions

Disclosed herein is a composition, comprising: one or more bacteria belonging to the Alistipes genus. Disclosed herein is a composition, comprising: one or more bacteria belonging to the Akkermansia genus. Disclosed herein is a composition, comprising: one or more bacteria belonging to the Alistipes genus and one or more bacteria belonging to the Akkermansia genus. Disclosed herein is a composition, comprising: one or more bacteria belonging to the Alistipes genus and one or more probiotics. Disclosed herein is a composition, comprising: one or more bacteria belonging to the Akkermansia genus and one or more probiotics. Disclosed herein is a composition, comprising: one or more bacteria belonging to the Alistipes genus and one or more bacteria belonging to the Akkermansia genus and one or more probiotics. Disclosed herein is a composition, comprising: one or more bacteria belonging to the Alistipes genus and one or more prebiotics. Disclosed herein is a composition, comprising: one or more bacteria belonging to the Akkermansia genus and one or more prebiotics. Disclosed herein is a composition, comprising: one or more bacteria belonging to the Alistipes genus and one or more bacteria belonging to the Akkermansia genus and one or more prebiotics.

Disclosed herein is a composition, comprising: one or more agents that can reduce the intestinal carriage associated with the colonization of drug-resistant E. coli bacteria, thereby reducing the risk of an infection. Disclosed herein is a composition, comprising: one or more agents that can reduce the proportional abundance of one or more bacteria associated with the carriage and/or persistence of drug-resistant E. coli bacteria, thereby reducing the degree and/or severity of colonization by a drug-resistance E. coli. Disclosed herein is a composition, comprising: one or more agents that can inhibit proliferation of Asaccharobacter, Bifidobacterium, Collinsella, Eggerthella, Gardnerella, Gordonibacter, or any combination thereof in the gut microbiome of the subject. Disclosed herein is a composition, comprising: one or more agents that can increase proliferation of Alistipes and/or Akkermansia.

In an aspect, the one or more disclosed bacteria belonging to the Alistipes genus can be isolated. In an aspect, the one or more bacteria belonging to the Alistipes genus can be pure or substantially pure. In an aspect, the one or more bacteria belonging to the Akkermansia genus can be isolated. In an aspect, the one or more bacteria belonging to the Akkermansia genus can be pure or substantially pure. In an aspect, the one or more bacteria belonging to the Alistipes genus can be isolated and viable. In an aspect, the one or more bacteria belonging to the Alistipes genus can be pure or substantially pure and viable. In an aspect, the one or more bacteria belonging to the Akkermansia genus can be isolated and viable. In an aspect, the one or more bacteria belonging to the Akkermansia genus can be pure or substantially pure and viable. In an aspect, the one or more bacteria belonging to the Alistipes genus can be recombinant and/or engineered. In an aspect, the one or more bacteria belonging to the Akkermansia genus can be recombinant and/or engineered.

In an aspect, a “prebiotic” refers to an ingredient that allows specific changes, both in the composition and/or activity in the gastrointestinal microbiota that may (or may not) confer benefits upon the host. In an aspect, a prebiotic can be a comestible food or beverage or ingredient thereof. In an aspect, a prebiotic may be a selectively fermented ingredient. Prebiotics may include complex carbohydrates, amino acids, peptides, minerals, or other essential nutritional components for the survival of the bacterial composition. Prebiotics include, but are not limited to, amino acids, biotin, fructooligosaccharide, galactooligosaccharides, hemicelluloses (e.g., arabinoxylan, xylan, xyloglucan, and glucomannan), inulin, chitin, lactulose, mannan oligosaccharides, oligofructose-enriched inulin, gums (e.g., guar gum, gum arabic and carregenaan), oligofructose, oligodextrose, tagatose, resistant maltodextrins (e.g., resistant starch), trans-galactooligosaccharide, pectins (e.g., xylogalactouronan, citrus pectin, apple pectin, and rhamnogalacturonan-I), dietary fibers (e.g., soy fiber, sugarbeet fiber, pea fiber, corn bran, and oat fiber) and xylooligosaccharides.

In an aspect, a disclosed composition can comprise one or more bacteriophages capable of killing E. coli. In an aspect, a disclosed E. coli-killing bacteriophage can comprise BP539, BP700, BP753, BP814, BP953, BP954, BP970, BP1002, BP1151, BP1155, BP1168, BP1176, BP1197, BP1226, BP1229, or any combination thereof. In an aspect, a disclosed E. coli-killing bacteriophage can comprise MV36.2, JJ2050.2, C3, C19T, JJ6.1, or any combination thereof.

In an aspect, a disclosed composition can (i) reduce the risk of developing a drug-resistant E. coli infection in a subject, (ii) reduce the risk of developing a colonization of drug-resistant E. coli in a subject, (iii) increase resistance to a drug-resistant E. coli infection and/or colonization in a subject, (iv) reduce the degree and/or severity of a drug-resistant E. coli infection in a subject, (v) reduce the degree and/or severity of a drug-resistant E. coli colonization in a subject, or (vi) any combination thereof.

In an aspect, a disclosed composition can increase and/or stimulate proliferation of one or more bacteria in the subject's gut microbiome and/or gastrointestinal tract. In an aspect, a disclosed composition can inhibit proliferation of one or more bacteria belonging to the Actinobacteria phylum in the subject's gut and/or gastrointestinal tract.

In an aspect, a disclosed composition can modulate the intestinal carriage and persistence of one or more bacteria. In an aspect, a disclosed composition can decrease and/or disrupt the intestinal carriage and persistence of one or more bacteria. In an aspect, a disclosed composition can inhibit proliferation of one or more bacteria in the subject's gut microbiome and/or gastrointestinal tract.

In an aspect, a disclosed composition can inhibit proliferation of one or more bacteria belonging to the Actinobacteria phylum in the subject's gut and/or gastrointestinal tract. In an aspect, a disclosed composition can inhibit proliferation of Asaccharobacter, Bifidobacterium, Collinsella, Eggerthella, Gardnerella, Gordonibacter, or any combination thereof in the subject's gut and/or gastrointestinal tract. In an aspect, a disclosed composition can inhibit proliferation of Collinsella in the subject's gut and/or gastrointestinal tract. In an aspect, a disclosed composition can inhibit proliferation of Gardnerella and/or Gordonibacter in the subject's gut and/or gastrointestinal tract. In an aspect, a disclosed composition can inhibit proliferation of one or more bacteria belonging to the Firmicutes phylum in the subject's gut and/or gastrointestinal tract. In an aspect, a disclosed composition can inhibit proliferation of Enterococcus, Lactobacillus, Streptococcus, Weissella, or any combination thereof in the subject's gut and/or gastrointestinal tract. In an aspect, a disclosed composition can inhibit proliferation of Lactobacillus and/or Streptococcus in the subject's gut and/or gastrointestinal tract.

In an aspect, a disclosed composition can increase and/or stimulate proliferation of one or more bacteria in the subject's gut microbiome and/or gastrointestinal tract. In an aspect, a disclosed composition can increase and/or stimulate proliferation of one or more bacteria belonging to the Bacteroidetes phylum in the subject's gut microbiome and/or gastrointestinal tract. In an aspect, a disclosed composition can increase and/or stimulate proliferation of one or more bacteria belonging to the Verrucomicrobia phylum in the subject's gut microbiome and/or gastrointestinal tract. In an aspect, a disclosed composition can that increase and/or stimulate proliferation of Alistipes and/or Akkermansia the subject's gut microbiome and/or gastrointestinal tract.

In an aspect, a disclosed composition can inhibit proliferation of one or more E. coli bacteria in the gastrointestinal tract of the subject. In an aspect, a disclosed composition can inhibit proliferation of one or more E. coli bacteria in the gut microbiome of the subject. In an aspect, a disclosed composition can inhibit proliferation of E. coli bacteria comprise sequence type 131 (ST131) in the gastrointestinal tract of the subject. In an aspect, a disclosed composition can inhibit proliferation of E. coli bacteria comprise sequence type 131 (ST131) in the gut microbiome of the subject. In an aspect, a disclosed composition can inhibit proliferation of H30R in the gastrointestinal tract of the subject. In an aspect, a disclosed composition can inhibit proliferation of H30R in the gut microbiome of the subject. In an aspect, a disclosed composition can inhibit proliferation of H30Rx in the gastrointestinal tract of the subject. In an aspect, a disclosed composition can inhibit proliferation of H30Rx in the gut microbiome of the subject.

In an aspect, a disclosed composition can inhibit proliferation of one or more other bacterial infections in one or more systems of a subject. In an aspect, a disclosed composition can inhibit proliferation of one or more other bacterial infections in one or more organs of a subject. In an aspect, a disclosed composition can decrease and/or disrupt the intestinal carriage and persistence of one or more bacteria. In an aspect, a disclosed composition can increase and/or stimulate proliferation of one or more bacteria in the subject's gut microbiome and/or gastrointestinal tract. In an aspect, a disclosed composition can increase and/or stimulate proliferation of one or more bacteria in the subject's gut microbiome and/or gastrointestinal tract. In an aspect, a disclosed composition can increase and/or stimulate proliferation of one or more bacteria belonging to the Bacteroidetes phylum in the subject's gut microbiome and/or gastrointestinal tract.

Disclosed herein is a pharmaceutical formulation comprising a disclosed composition and at least one pharmaceutically acceptable carrier.

Disclosed herein is a pharmaceutical formulation comprising one or more bacteria belonging to the Alistipes genus and at least one pharmaceutically acceptable carrier. Disclosed herein is a pharmaceutical formulation comprising one or more bacteria belonging to the Akkermansia genus and at least one pharmaceutically acceptable carrier. Disclosed herein is a pharmaceutical formulation comprising one or more bacteria belonging to the Alistipes genus and one or more bacteria belonging to the Akkermansia genus and at least one pharmaceutically acceptable carrier. Disclosed herein is a pharmaceutical formulation comprising one or more bacteria belonging to the Alistipes genus and one or more probiotics and at least one pharmaceutically acceptable carrier. Disclosed herein is a pharmaceutical formulation comprising one or more bacteria belonging to the Akkermansia genus and one or more probiotics and at least one pharmaceutically acceptable carrier. Disclosed herein is a pharmaceutical formulation comprising one or more bacteria belonging to the Alistipes genus and one or more bacteria belonging to the Akkermansia genus and one or more probiotics and at least one pharmaceutically acceptable carrier. Disclosed herein is a pharmaceutical formulation comprising one or more bacteria belonging to the Alistipes genus and one or more prebiotics and at least one pharmaceutically acceptable carrier. Disclosed herein is a pharmaceutical formulation comprising one or more bacteria belonging to the Akkermansia genus and one or more prebiotics and at least one pharmaceutically acceptable carrier. Disclosed herein is a pharmaceutical formulation comprising one or more bacteria belonging to the Alistipes genus and one or more bacteria belonging to the Akkermansia genus and one or more prebiotics and at least one pharmaceutically acceptable carrier.

Disclosed herein is a pharmaceutical formulation comprising one or more bacteriophages capable of killing E. coli. In an aspect of a disclosed pharmaceutical formulation, a disclosed E. coli-killing bacteriophage can comprise BP539, BP700, BP753, BP814, BP953, BP954, BP970, BP1002, BP1151, BP1155, BP1168, BP1176, BP1197, BP1226, BP1229, or any combination thereof. In an aspect of a disclosed pharmaceutical formulation, a disclosed E. coli-killing bacteriophage can comprise MV36.2, JJ2050.2, C3, C19T, JJ6.1, or any combination thereof.

Disclosed herein is a pharmaceutical formulation comprising one or more agents that can reduce the intestinal carriage associated with the colonization of drug-resistant E. coli bacteria and at least one pharmaceutically acceptable carrier. Disclosed herein is a pharmaceutical formulation comprising one or more agents that can reduce the proportional abundance of one or more bacteria associated with the carriage and/or persistence of drug-resistant E. coli bacteria and at least one pharmaceutically acceptable carrier. Disclosed herein is a pharmaceutical formulation comprising one or more agents that can inhibit proliferation of Asaccharobacter, Bifidobacterium, Collinsella, Eggerthella, Gardnerella, Gordonibacter, or any combination thereof in the gut microbiome of the subject and at least one pharmaceutically acceptable carrier. Disclosed herein is a pharmaceutical formulation comprising one or more agents that can increase proliferation of Alistipes and/or Akkermansia and at least one pharmaceutically acceptable carrier.

In an aspect, a disclosed pharmaceutical formulation can (i) reduce the risk of developing a drug-resistant E. coli infection in a subject, (ii) reduce the risk of developing a colonization of drug-resistant E. coli in a subject, (iii) increase resistance to a drug-resistant E. coli infection and/or colonization in a subject, (iv) reduce the degree and/or severity of a drug-resistant E. coli infection in a subject, (v) reduce the degree and/or severity of a drug-resistant E. coli colonization in a subject, or (vi) any combination thereof.

In an aspect, a disclosed pharmaceutical formulation can increase and/or stimulate proliferation of one or more bacteria in the subject's gut microbiome and/or gastrointestinal tract. In an aspect, a disclosed pharmaceutical formulation can inhibit proliferation of one or more bacteria belonging to the Actinobacteria phylum in the subject's gut and/or gastrointestinal tract.

In an aspect, a disclosed pharmaceutical formulation can modulate the intestinal carriage and persistence of one or more bacteria. In an aspect, a disclosed pharmaceutical formulation can decrease and/or disrupt the intestinal carriage and persistence of one or more bacteria. In an aspect, a disclosed pharmaceutical formulation can inhibit proliferation of one or more bacteria in the subject's gut microbiome and/or gastrointestinal tract.

In an aspect, a disclosed pharmaceutical formulation can inhibit proliferation of one or more bacteria belonging to the Actinobacteria phylum in the subject's gut and/or gastrointestinal tract. In an aspect, a disclosed composition can inhibit proliferation of Asaccharobacter, Bifidobacterium, Collinsella, Eggerthella, Gardnerella, Gordonibacter, or any combination thereof in the subject's gut and/or gastrointestinal tract. In an aspect, a disclosed composition can inhibit proliferation of Collinsella in the subject's gut and/or gastrointestinal tract. In an aspect, a disclosed pharmaceutical formulation can inhibit proliferation of Gardnerella and/or Gordonibacter in the subject's gut and/or gastrointestinal tract. In an aspect, a disclosed pharmaceutical formulation can inhibit proliferation of one or more bacteria belonging to the Firmicutes phylum in the subject's gut and/or gastrointestinal tract. In an aspect, a disclosed pharmaceutical formulation can inhibit proliferation of Enterococcus, Lactobacillus, Streptococcus, Weissella, or any combination thereof in the subject's gut and/or gastrointestinal tract. In an aspect, a disclosed pharmaceutical formulation can inhibit proliferation of Lactobacillus and/or Streptococcus in the subject's gut and/or gastrointestinal tract.

In an aspect, a disclosed pharmaceutical formulation can increase and/or stimulate proliferation of one or more bacteria in the subject's gut microbiome and/or gastrointestinal tract. In an aspect, a disclosed pharmaceutical formulation can increase and/or stimulate proliferation of one or more bacteria belonging to the Bacteroidetes phylum in the subject's gut microbiome and/or gastrointestinal tract. In an aspect, a disclosed pharmaceutical formulation can increase and/or stimulate proliferation of one or more bacteria belonging to the Verrucomicrobia phylum in the subject's gut microbiome and/or gastrointestinal tract. In an aspect, a disclosed pharmaceutical formulation can that increase and/or stimulate proliferation of Alistipes and/or Akkermansia the subject's gut microbiome and/or gastrointestinal tract.

In an aspect, a disclosed pharmaceutical formulation can inhibit proliferation of one or more E. coli bacteria in the gastrointestinal tract of the subject. In an aspect, a disclosed pharmaceutical formulation can inhibit proliferation of one or more E. coli bacteria in the gut microbiome of the subject. In an aspect, a disclosed pharmaceutical formulation can inhibit proliferation of E. coli bacteria comprise sequence type 131 (ST131) in the gastrointestinal tract of the subject. In an aspect, a disclosed pharmaceutical formulation can inhibit proliferation of E. coli bacteria comprise sequence type 131 (ST131) in the gut microbiome of the subject. In an aspect, a disclosed pharmaceutical formulation can inhibit proliferation of H30R in the gastrointestinal tract of the subject. In an aspect, a disclosed pharmaceutical formulation can inhibit proliferation of H30R in the gut microbiome of the subject. In an aspect, a disclosed pharmaceutical formulation can inhibit proliferation of H30Rx in the gastrointestinal tract of the subject. In an aspect, a disclosed pharmaceutical formulation can inhibit proliferation of H30Rx in the gut microbiome of the subject.

In an aspect, a disclosed pharmaceutical formulation can inhibit proliferation of one or more other bacterial infections in one or more systems of a subject. In an aspect, a disclosed pharmaceutical formulation can inhibit proliferation of one or more other bacterial infections in one or more organs of a subject. In an aspect, a disclosed pharmaceutical formulation can decrease and/or disrupt the intestinal carriage and persistence of one or more bacteria. In an aspect, a disclosed pharmaceutical formulation can increase and/or stimulate proliferation of one or more bacteria in the subject's gut microbiome and/or gastrointestinal tract. In an aspect, a disclosed pharmaceutical formulation can increase and/or stimulate proliferation of one or more bacteria in the subject's gut microbiome and/or gastrointestinal tract. In an aspect, a disclosed pharmaceutical formulation can increase and/or stimulate proliferation of one or more bacteria belonging to the Bacteroidetes phylum in the subject's gut microbiome and/or gastrointestinal tract. In an aspect, a disclosed pharmaceutical formulation can further comprise a growth medium to sustain a disclosed probiotic prior to administration to the subject.

In an aspect of a disclosed pharmaceutical formulation, a disclosed probiotic can be lyophilized or freeze-dried. In an aspect of a disclosed pharmaceutical formulation, a disclosed consortium of probiotics can be lyophilized or freeze-dried.

In an aspect, a disclosed pharmaceutical formulation can comprise at least one lyoprotectant. In an aspect, a disclosed lyoprotectant can comprise peptone, glycerol, lactose, gelatin, glucose, sucrose, trehalose, dextran, maltodextrin, adonitol, sodium glutamate, or any combination thereof. Lyoprotectants are known to those skilled in the art.

In an aspect, a disclosed pharmaceutical formulation can comprise at least one gelling agent, preferably a pharmaceutically acceptable gelling agent. In an aspect, a disclosed pharmaceutical formulation can comprise at least preservative such as, for example, benzyl alcohol, cresols, benzoic acid, phenol, parabens, or sorbic acid. In an aspect, a disclosed pharmaceutical formulation can comprise at least one stabilizer such as, for example, a surfactant, a polymer, a polyol, a poloxamer, an albumin, a gelatin, a trehalose, a protein, a sugar, a polyvinylpyrrolidone, a N-acetyl-tryptophan (NAT), a caprylate (e.g., sodium caprylate), a polysorbate (e.g., P80), an amino acid, and a divalent metal cation (e.g., zinc).

In an aspect of a disclosed pharmaceutical formulation, a disclosed biotherapeutic can comprise one or more probiotics. In an aspect of a disclosed pharmaceutical formulation, a disclosed consortium of probiotics can comprise at least one strain from the bacterial genus Alistipes and/or at least one strain from the bacterial genus Akkermansia.

In an aspect of a disclosed pharmaceutical formulation, a disclosed biotherapeutic can comprise one or more factors secreted by a disclosed probiotic or secreted by a disclosed consortium of probiotics. In an aspect of a disclosed pharmaceutical formulation, a disclosed biotherapeutic can comprise one or more factors secreted by at least one strain from the bacterial genus Alistipes and/or at least one strain from the bacterial genus Akkermansia. In an aspect of a disclosed pharmaceutical formulation, a disclosed biotherapeutic can comprise one or more factors secreted by at least one strain from the bacterial genus Alistipes and/or at least one strain from the bacterial genus Akkermansia. In an aspect of a disclosed pharmaceutical formulation, a disclosed biotherapeutic can comprise one or more factors secreted by at least one strain from the bacterial genus Alistipes and/or at least one strain from the bacterial genus Akkermansia. In an aspect of a disclosed pharmaceutical formulation, a disclosed biotherapeutic can comprise one or more factors secreted by at least one strain from the bacterial genus Alistipes and/or at least one strain from the bacterial genus Akkermansia. In an aspect of a disclosed pharmaceutical formulation, a disclosed biotherapeutic can comprise one or more factors secreted by at least one strain from the bacterial genus Alistipes and/or at least one strain from the bacterial genus Akkermansia. In an aspect of a disclosed pharmaceutical formulation, a disclosed biotherapeutic can comprise one or more factors secreted by at least one strain from the bacterial genus Alistipes and/or at least one strain from the bacterial genus Akkermansia. In an aspect of a disclosed pharmaceutical formulation, a disclosed biotherapeutic can comprise comprises one or more factors secreted by at least one strain from the bacterial genus Alistipes and/or at least one strain from the bacterial genus Akkermansia. In an aspect of a disclosed pharmaceutical formulation, a disclosed biotherapeutic can comprise one or more factors secreted by at least one strain from the bacterial genus Alistipes and/or at least one strain from the bacterial genus Akkermansia.

In an aspect of a disclosed pharmaceutical formulation, a disclosed therapeutically effective dose can comprise a total of at least 100 bacterial CFUs of the one or more probiotics, a total of at least 200 to at least 300 bacterial CFUs of the one or more probiotics, a total of at least 300 to at least 400 bacterial CFUs of the one or more probiotics, a total of at least 400 to at least 500 bacterial CFUs of the one or more probiotics, a total of at least 500 to at least 600 bacterial CFUs of the one or more probiotics, a total of at least 600 to at least 700 bacterial CFUs of the one or more probiotics, a total of at least 700 to at least 800 bacterial CFUs of the one or more probiotics, a total of at least 800 to at least 900 bacterial CFUs of the one or more probiotics, a total of at least 900 to at least 1000 bacterial CFUs of the one or more probiotics, or a total of at least 1000 bacterial CFUs of one or more disclosed probiotics (such as, for example, at least one strain from the bacterial genus Alistipes and/or at least one strain from the bacterial genus Akkermansia).

In an aspect of a disclosed pharmaceutical formulation, a disclosed therapeutically effective dose can comprise a total of at least 1000 to at least 2000 bacterial CFUs of the one or more probiotics, a total of at least 2000 to at least 3000 bacterial CFUs of the one or more probiotics, a total of at least 3000 to at least 4000 bacterial CFUs of the one or more probiotics, a total of at least 4000 to at least 5000 bacterial CFUs of the one or more probiotics, a total of at least 5000 to at least 6000 bacterial CFUs of the one or more probiotics, a total of at least 6000 to at least 7000 bacterial CFUs of the one or more probiotics, a total of at least 8000 to at least 9000 bacterial CFUs of the one or more probiotics, or a total of at least 10,000 bacterial CFUs of one or more disclosed probiotics (such as, for example, at least one strain from the bacterial genus Alistipes and/or at least one strain from the bacterial genus Akkermansia).

In an aspect of a disclosed pharmaceutical formulation, a disclosed therapeutically effective dose can comprise a total of at least 100 bacterial CFUs of at least one strain from the bacterial genus Alistipes and/or at least one strain from the bacterial genus Akkermansia, a total of at least 200 to at least 300 bacterial CFUs of at least one strain from the bacterial genus Alistipes and/or at least one strain from the bacterial genus Akkermansia, a total of at least 300 to at least 400 bacterial CFUs of at least one strain from the bacterial genus Alistipes and/or at least one strain from the bacterial genus Akkermansia, a total of at least 400 to at least 500 bacterial CFUs of at least one strain from the bacterial genus Alistipes and/or at least one strain from the bacterial genus Akkermansia, a total of at least 500 to at least 600 bacterial CFUs of at least one strain from the bacterial genus Alistipes and/or at least one strain from the bacterial genus Akkermansia, a total of at least 600 to at least 700 bacterial CFUs of at least one strain from the bacterial genus Alistipes and/or at least one strain from the bacterial genus Akkermansia, a total of at least 700 to at least 800 bacterial CFUs of at least one strain from the bacterial genus Alistipes and/or at least one strain from the bacterial genus Akkermansia, a total of at least 800 to at least 900 bacterial CFUs of at least one strain from the bacterial genus Alistipes and/or at least one strain from the bacterial genus Akkermansia, a total of at least 900 to at least 1000 bacterial CFUs of at least one strain from the bacterial genus Alistipes and/or at least one strain from the bacterial genus Akkermansia, or a total of at least 1000 bacterial CFUs of at least one strain from the bacterial genus Alistipes and/or at least one strain from the bacterial genus Akkermansia.

In an aspect of a disclosed pharmaceutical formulation, a disclosed therapeutically effective dose can comprise a total of at least 1000 to at least 2000 bacterial CFUs of at least one strain from the bacterial genus Alistipes and/or at least one strain from the bacterial genus Akkermansia, a total of at least 2000 to at least 3000 bacterial CFUs of at least one strain from the bacterial genus Alistipes and/or at least one strain from the bacterial genus Akkermansia, a total of at least 3000 to at least 4000 bacterial CFUs of at least one strain from the bacterial genus Alistipes and/or at least one strain from the bacterial genus Akkermansia, a total of at least 4000 to at least 5000 bacterial CFUs of at least one strain from the bacterial genus Alistipes and/or at least one strain from the bacterial genus Akkermansia, a total of at least 5000 to at least 6000 bacterial CFUs of at least one strain from the bacterial genus Alistipes and/or at least one strain from the bacterial genus Akkermansia, a total of at least 6000 to at least 7000 bacterial CFUs of at least one strain from the bacterial genus Alistipes and/or at least one strain from the bacterial genus Akkermansia, a total of at least 8000 to at least 9000 bacterial CFUs of at least one strain from the bacterial genus Alistipes and/or at least one strain from the bacterial genus Akkermansia, or a total of at least 10,000 bacterial CFUs of at least one strain from the bacterial genus Alistipes and/or at least one strain from the bacterial genus Akkermansia.

In an aspect, a disclosed therapeutically effective dose can comprise a total of at least 10⁴ to at least 10⁵ bacterial CFUs of the one or more probiotics, a total of at least 10⁵ to at least 10⁶ bacterial CFUs of the one or more probiotics, a total of at least 10⁶ to at least 10⁷ bacterial CFUs of the one or more probiotics, a total of at least 10⁷ to at least 10⁸ bacterial CFUs of the one or more probiotics, a total of at least 10⁸ to at least 10⁹ bacterial CFUs of the one or more probiotics, a total of at least 10⁹ to at least 10¹⁰ bacterial CFUs of the one or more probiotics, a total of at least 10¹⁰ to at least 10¹¹ bacterial CFUs of the one or more probiotics, a total of at least 10¹¹ to at least 10¹² bacterial CFUs of the one or more probiotics, a total of at least 10¹² bacterial CFUs of the one or more probiotics, or more than 10¹² bacterial CFUs of one or more disclosed probiotics (such as, for example, at least one strain from the bacterial genus Alistipes and/or at least one strain from the bacterial genus Akkermansia).

In an aspect of a disclosed pharmaceutical formulation, a disclosed therapeutically effective dose can comprise at least 10³, 10⁴, 10⁵, 10⁶, 10⁷, 10⁸, 10⁹, 10¹⁰, 10¹¹, or 10¹² CFUs of a disclosed probiotic or a consortium of disclosed probiotics, preferably at least 1.2×10³, 1.4×10³, 2×10³, or 3×10³ CFUs of a disclosed probiotic or a consortium of disclosed probiotics, at least 5×10³, 5.02×10³, 5.04×10³, 5.2×10³, or 5.4×10³ CFUs of a disclosed probiotic or a consortium of disclosed probiotics, at least 1.02×10⁴, 1.04×10⁴, 1.2×10⁴, 1.4×10⁴, 1.5×10⁴, 2×10⁴, or 3×10⁴ CFUs of a disclosed probiotic or a consortium of disclosed probiotics, at least 5×10⁴, 5.02×10⁴, 5.04×10⁴, 5.2×10⁴, or 5.4×10⁴ CFUs of a disclosed probiotic or a consortium of disclosed probiotics, at least 1.02×10⁵, 1.04×10⁵, 1.2×10⁵, 1.4×10⁵, 1.5×10⁵, 2×10⁵, or 3×10⁵ CFUs of a disclosed probiotic or a consortium of disclosed probiotics, at least 1.02×10⁶, 1.04×10⁶, 1.2×10⁶, 1.4×10⁶, 2×10⁶, or 3×10⁶ CFUs of a disclosed probiotic or a consortium of disclosed probiotics, at least 1.02×10⁷, 1.04×10⁷, 1.2×10⁷, 1.4×10⁷, 2×10⁷, or 3×10⁷ CFUs of a disclosed probiotic or a consortium of disclosed probiotics, at least 1.02×10⁸, 1.04×10⁸, 1.2×10⁸, 1.4×10⁸, 2×10⁸, or 3×10⁸ CFUs of a disclosed probiotic or a consortium of disclosed probiotics, at least 1.02×10⁹, 1.04×10⁹, 1.2×10⁹, 1.4×10⁹, 2×10⁹, or 3×10⁹ CFUs of a disclosed probiotic or a consortium of disclosed probiotics, at least 10¹⁰ CFUs of a disclosed probiotic or a consortium of disclosed probiotics, at least 1.02×10¹⁰, 1.04×10¹⁰, 1.2×10¹⁰, 1.4×10¹⁰, 2×10¹⁰, or 3×10¹⁰ CFUs of a disclosed probiotic or a consortium of disclosed probiotics, at least 1.02×10¹¹, 1.04×10¹¹, 1.2×10¹¹, 1.4×10¹¹, 2×10¹¹, or 3×10¹¹ CFUs of a disclosed probiotic or a consortium of disclosed probiotics, or at least 1.02×10¹², 1.04×10¹², 1.2×10¹², 1.4×10¹², 2×10¹², or 3×10¹² CFUs of a disclosed probiotic or a consortium of disclosed probiotics per dose.

In an aspect, a disclosed therapeutically effective dose can comprise any amount or an unlimited amount of bacterial CFUs of the one or more probiotics.

In an aspect, a disclosed pharmaceutical formulation can be prepared for systemic or direct administration. In an aspect, a disclosed pharmaceutical formulation can be prepared for oral administration, intravenous administration, intranasal administration, sublingual administration, intraperitoneal administration, or any combination thereof. In an aspect, a disclosed pharmaceutical formulation can be prepared for any method of administration disclosed herein. In an aspect, a disclosed pharmaceutical formulation can be prepared for administration via multiple routes either concurrently or sequentially.

In an aspect, a disclosed pharmaceutical formulation can further comprise (i) one or more active agents, (ii) one or more biologically active agents, (iii) one or more pharmaceutically active agents, (iv) one or more immune-based therapeutic agents, (v) one or more clinically approved agents, (vi) one or more anti-fungal agents, (vii) one or more anti-bacterial agents, (viii) one or more corticosteroids, (ix) one or more analgesics, (x) one or more anti-viral agents, (xi) one or more immunostimulants, or (xii) any combination thereof. In an aspect, a disclosed pharmaceutical formulation can comprise one or more anti-bacterial agents. In an aspect of a disclosed pharmaceutical formulation, the dose of an active agent, a biologically active agent, a pharmaceutically active agent, an immune-based therapeutic active agent, a clinically approved agent, or an anti-bacterial agent can be about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

### Biologically Active Agents

In an aspect, the term “biologically active agent” or “biologic active agent” or “bioactive agent” means an agent that is capable of providing a local or systemic biological, physiological, or therapeutic effect in the biological system to which it is applied. For example, the bioactive agent can act to control infection or inflammation, enhance cell growth and tissue regeneration, control tumor growth, act as an analgesic, promote anti-cell attachment, and enhance bone growth, among other functions. Other suitable bioactive agents can include anti-viral agents, vaccines, hormones, antibodies (including active antibody fragments sFv, Fv, and Fab fragments), aptamers, peptide mimetics, functional nucleic acids, therapeutic proteins, peptides, or nucleic acids. Other bioactive agents include prodrugs, which are agents that are not biologically active when administered but, upon administration to a subject are converted to bioactive agents through metabolism or some other mechanism. Additionally, any of the compositions of the invention can contain combinations of two or more bioactive agents. It is understood that a biologically active agent can be used in connection with administration to various subjects, for example, to humans (i.e., medical administration) or to animals (i.e., veterinary administration). In an aspect, the recitation of a biologically active agent inherently encompasses the pharmaceutically acceptable salts thereof.

### Pharmaceutically Active Agents

In an aspect, the term “pharmaceutically active agent” includes a “drug” or a “vaccine” and means a molecule, group of molecules, complex or substance administered to an organism for diagnostic, therapeutic, preventative medical, or veterinary purposes. This term includes externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA) or ribonucleic acid (RNA). Pharmaceutically active agents include the herein disclosed categories and specific examples. It is not intended that the category be limited by the specific examples. Those of ordinary skill in the art will recognize also numerous other compounds that fall within the categories and that are useful according to the invention. Examples include a radiosensitizer, the combination of a radiosensitizer and a chemotherapeutic, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, carbonic anhydrase inhibitors, prostaglandin analogs, a combination of an alpha agonist and a beta blocker, a combination of a carbonic anhydrase inhibitor and a beta blocker, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, or a vaccine. In a further aspect, the pharmaceutically active agent can be coumarin, albumin, bromolidine, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetominophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, timol hemihydrate, levobunolol hydrochloride, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists (i.e., alpha adrenergic receptor agonist) such as clonidine, brimonidine tartrate, and apraclonidine hydrochloride; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; prostaglandin analogs such as latanoprost, travoprost, and bimatoprost; cholinergics (i.e., acetylcholine receptor agonists) such as pilocarpine hydrochloride and carbachol; glutamate receptor agonists such as the N-methyl D-aspartate receptor agonist memantine; anti-Vascular endothelial growth factor (VEGF) aptamers such as pegaptanib; anti-VEGF antibodies (including but not limited to anti-VEGF-A antibodies) such as ranibizumab and bevacizumab; carbonic anhydrase inhibitors such as methazolamide, brinzolamide, dorzolamide hydrochloride, and acetazolamide; antiarrhythmic agents such as quinidine, lidocaine, tocainide hydrochloride, mexiletine hydrochloride, digoxin, verapamil hydrochloride, propafenone hydrochloride, flecaimide acetate, procainamide hydrochloride, moricizine hydrochloride, and diisopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as coumadin, warfarin, acetylsalicylic acid, and ticlopidine; sedatives such as benzodiazepines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hydrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides. It is understood that a pharmaceutically active agent can be used in connection with administration to various subjects, for example, to humans (i.e., medical administration) or to animals (i.e., veterinary administration). In an aspect, the recitation of a pharmaceutically active agent inherently encompasses the pharmaceutically acceptable salts thereof.

### Anti-Bacterial Agents

In an aspect, anti-bacterial agents are known to the art. For example, the art generally recognizes several categories of anti-bacterial agents including (1) penicillins, (2) cephalosporins, (3) quinolones, (4) aminoglycosides, (5) monobactams, (6) carbapenems, (7) macrolides, and (8) other agents. For example, In an aspect, an anti-bacterial agent can comprise Afenide, Amikacin, Amoxicillin, Ampicillin, Arsphenamine, Augmentin, Azithromycin, Azlocillin, Aztreonam, Bacampicillin, Bacitracin, Balofloxacin, Besifloxacin, Capreomycin, Carbacephem (loracarbef), Carbenicillin, Cefacetrile (cephacetrile), Cefaclomezine, Cefaclor, Cefadroxil (cefadroxyl), Cefalexin (cephalexin), Cefaloglycin (cephaloglycin), Cefalonium (cephalonium), Cefaloram, Cefaloridine (cephaloradine), Cefalotin (cephalothin), Cefamandole, Cefaparole, Cefapirin (cephapirin), Cefatrizine, Cefazaflur, Cefazedone, Cefazolin (cephazolin), Cefcanel, Cefcapene, Cefclidine, Cefdaloxime, Cefdinir, Cefditoren, Cefedrolor, Cefempidone, Cefepime, Cefetamet, Cefetrizole, Cefivitril, Cefixime, Cefluprenam, Cefmatilen, Cefmenoxime, Cefmepidium, Cefmetazole, Cefodizime, Cefonicid, Cefoperazone, Cefoselis, Cefotaxime, Cefotetan, Cefovecin, Cefoxazole, Cefoxitin, Cefozopran, Cefpimizole, Cefpirome, Cefpodoxime, Cefprozil (cefproxil), Cefquinome, Cefradine (cephradine), Cefrotil, Cefroxadine, Cefsumide, Ceftaroline, Ceftazidime, Ceftazidime/Avibactam, Cefteram, Ceftezole, Ceftibuten, Ceftiofur, Ceftiolene, Ceftioxide, Ceftizoxime, Ceftobiprole, Ceftriaxone, Cefuracetime, Cefuroxime, Cefuzonam, Cephalexin, Chloramphenicol, Chlorhexidine, Ciprofloxacin, Clarithromycin, Clavulanic Acid, Clinafloxacin, Clindamycin, Cloxacillin, Colimycin, Colistimethate, Colistin, Crysticillin, Cycloserine 2, Demeclocycline, Dicloxacillin, Dirithromycin, Doripenem, Doxycycline, Efprozil, Enoxacin, Ertapenem, Erythromycin, Ethambutol, Flucloxacillin, Flumequine, Fosfomycin, Furazolidone, Gatifloxacin, Geldanamycin, Gemifloxacin, Gentamicin, Glycopeptides, Grepafloxacin, Herbimycin, Imipenem, Isoniazid, Kanamycin, Levofloxacin, Lincomycin, Linezolid, Lipoglycopeptides, Lomefloxacin, Meropenem, Meticillin, Metronidazole, Mezlocillin, Minocycline, Mitomycin, Moxifloxacin, Mupirocin, Nadifloxacin, Nafcillin, Nalidixic Acid, Neomycin, Netilmicin, Nitrofurantoin, Norfloxacin, Ofloxacin, Oxacillin, Oxazolidinones, Oxolinic Acid, Oxytetracycline, Oxytetracycline, Paromomycin, Pazufloxacin, Pefloxacin, Penicillin G, Penicillin V, Pipemidic Acid, Piperacillin, Piromidic Acid, Pivampicillin, Pivmecillinam, Platensimycin, Polymyxin B, Pristinamycin, Prontosil, Prulifloxacin, Pvampicillin, Pyrazinamide, Quinupristin/dalfopristin, Rifabutin, Rifalazil, Rifampin, Rifamycin, Rifapentine, Rosoxacin, Roxithromycin, Rufloxacin, Sitafloxacin, Sparfloxacin, Spectinomycin, Spiramycin, Streptomycin, Sulbactam, Sulfacetamide, Sulfamethizole, Sulfamethoxazole, Sulfanilimide, Sulfisoxazole, Sulphonamides, Sultamicillin, Teicoplanin, Telavancin, Telithromycin, Temafloxacin, Tetracycline, Thiamphenicol, Ticarcillin, Tigecycline, Tinidazole, Tobramycin, Tosufloxacin, Trimethoprim, Trimethoprim-Sulfamethoxazole, Troleandomycin, Trovafloxacin, Tuberactinomycin, Vancomycin, Viomycin, or pharmaceutically acceptable salts thereof (e.g., such as, for example, chloride, bromide, iodide, and periodate), or a combination thereof. In an aspect, the recitation of an anti-bacterial agent inherently encompasses the pharmaceutically acceptable salts thereof.

### Anti-Fungal Agents

Anti-fungal agents are known to the art. The art generally recognizes several categories of anti-fungal agents including (1) azoles (imidazoles), (2) antimetabolites, (3) allylamines, (4) morpholine, (5) glucan synthesis inhibitors (echinocandins), (6) polyenes, (7) benoxaaborale; (8) other antifungal/onychomycosis agents, and (9) new classes of antifungal/onychomycosis agents. For example, in an aspect, an anti-fungal agent can comprise Abafungin, Albaconazole, Amorolfin, Amphotericin B, Anidulafungin, Bifonazole, Butenafine, Butoconazole, Candicidin, Caspofungin, Ciclopirox, Clotrimazole, Econazole, Fenticonazole, Filipin, Fluconazole, Flucytosine, Griseofulvin, Haloprogin, Hamycin, Isavuconazole, Isoconazole, Itraconazole, Ketoconazole, Micafungin, Miconazole, Naftifine, Natamycin, Nystatin, Omoconazole, Oxiconazole, Polygodial, Posaconazole, Ravuconazole, Rimocidin, Sertaconazole, Sulconazole, Terbinafine, Terconazole, Tioconazole, Tolnaftate, Undecylenic Acid, Voriconazole, or pharmaceutically acceptable salts thereof, or a combination thereof. In an aspect, an anti-fungal agent can be an azole. Azoles include, but are not limited to, the following: clotrimazole, econazole, fluconazole, itraconazole, ketoconazole, miconazole, oxiconazole, sulconazole, and voriconazole. In an aspect, the recitation of an anti-fungal agent inherently encompasses the pharmaceutically acceptable salts thereof.

### Anti-Viral Agents

Anti-viral agents are known to the art. In an aspect, for example, an anti-viral can comprise Abacavir, Acyclovir (Aciclovir), Adefovir, Amantadine, Ampligen, Amprenavir (Agenerase), Umifenovir (Arbidol), Atazanavir, Atripla, Baloxavir marboxil (Xofluza), Biktarvy, Boceprevir, Bulevirtide, Cidofovir, Cobicistat (Tybost), Combivir, Daclatasvir (Daklinza), Darunavir, Delavirdine, Descovy, Didanosine, Docosanol, Dolutegravir, Doravirine (Pifeltro), Edoxudine, Efavirenz, Elvitegravir, Emtricitabine, Enfuvirtide, Entecavir, Etravirine (Intelence), Famciclovir, Fomivirsen, Fosamprenavir, Foscarnet, Ganciclovir (Cytovene), Ibacitabine, Ibalizumab (Trogarzo), Idoxuridine, Imiquimod, Imunovir, Indinavir, Lamivudine, Letermovir (Prevymis), Lopinavir, Loviride, Maraviroc, Methisazone, Moroxydine, Nelfinavir, Nevirapine, Nexavir (formerly Kutapressin), Nitazoxanide, Norvir, Oseltamivir (Tamiflu), Penciclovir, Peramivir, Penciclovir, Peramivir (Rapivab), Pleconaril, Podophyllotoxin, Raltegravir, Remdesivir, Ribavirin, Rilpivirine (Edurant), Rilpivirine, Rimantadine, Ritonavir, Saquinavir, Simeprevir (Olysio), Sofosbuvir, Stavudine, Taribavirin (Viramidine), Telaprevir, Telbivudine (Tyzeka), Tenofovir alafenamide, Tenofovir disoproxil, Tenofovir, Tipranavir, Trifluridine, Trizivir, Tromantadine, Truvada, Umifenovirk, Valaciclovir, Valganciclovir (Valtrex), Vicriviroc, Vidarabine, Zalcitabine, Zanamivir (Relenza), Zidovudine, and combinations thereof. In an aspect, the recitation of any anti-viral agent inherently encompasses the pharmaceutically acceptable salts thereof.

### Corticosteroids

Corticosteroids are well-known in the art. Corticosteroids mimic the effects of hormones that the body produces naturally in your adrenal glands. Corticosteroids can suppress inflammation and can reduce the signs and symptoms of inflammatory conditions (e.g., arthritis and asthma). Corticosteroids can also suppress the immune system. Corticosteroids can act on a number of different cells (e.g., mast cells, neutrophils, macrophages and lymphocytes) and a number of different mediators (e.g., histamine, leukotriene, and cytokine subtypes).

Steroids include, but are not limited to, the following: triamcinolone and its derivatives (e.g., diacetate, hexacetonide, and acetonide), betamethasone and its derivatives (e.g., dipropionate, benzoate, sodium phosphate, acetate, and valerate), dexamethasone and its derivatives (e.g., dipropionate and valerate), flunisolide, prednisone and its derivatives (e.g., acetate), prednisolone and its derivatives (e.g., acetate, sodium phosphate, and tebutate), methylprednisolone and its derivatives (e.g., acetate and sodium succinate), fluocinolone and its derivatives (e.g., acetonide), diflorasone and its derivatives (e.g., diacetate), halcinonide, desoximetasone (desoxymethasone), diflucortolone and its derivatives (e.g., valerate), flucloronide (fluclorolone acetonide), fluocinonide, fluocortolone, fluprednidene and its derivatives (e.g., acetate), flurandrenolide (flurandrenolone), clobetasol and its derivatives (e.g., propionate), clobetasone and its derivatives (e.g., butyrate), alclometasone, flumethasone and its derivatives (e.g., pivalate), fluocortolone and its derivatives (e.g., hexanoate), amcinonide, beclometasone and its derivatives (e.g., dipropionate), fluticasone and its derivatives (e.g., propionate), difluprednate, prednicarbate, flurandrenolide, mometasone, and desonide. In an aspect, the recitation of a corticosteroid inherently encompasses the pharmaceutically acceptable salts thereof.

### Analgesics

The compositions of the present disclosure can also be used in combination therapies with opioids and other analgesics, including narcotic analgesics, Mu receptor antagonists, Kappa receptor antagonists, non-narcotic (i.e., non-addictive) analgesics, monoamine uptake inhibitors, adenosine regulating agents, cannabinoid derivatives, Substance P antagonists, neurokinin-1 receptor antagonists and sodium channel blockers, among others. Preferred combination therapies comprise a composition useful in methods described herein with one or more compounds selected from aceclofenac, acemetacin, .alpha.-acetamidocaproic acid, acetaminophen, acetaminosalol, acetanilide, acetylsalicylic acid (aspirin), S-adenosylmethionine, alclofenac, alfentanil, allylprodine, alminoprofen, aloxiprin, alphaprodine, aluminum bis(acetylsalicylate), amfenac, aminochlorthenoxazin, 3-amino-4-hydroxybutyric acid, 2-atnino-4-picoline, aminopropylon, aminopyrine, amixetrine, ammonium salicylate, ampiroxicam, amtolmetin guacil, anileridine, antipyrine, antipyrine salicylate, antrafenine, apazone, bendazac, benorylate, benoxaprofen, benzpiperylon, benzydamine, benzylmorphine, bermoprofen, bezitramide, .alpha.-bisabolol, bromfenac, p-bromoacetanilide, 5-bromosalicylic acid acetate, bromosaligenin, bucetin, bucloxic acid, bucolome, bufexamac, bumadizon, buprenorphine, butacetin, butibufen, butophanol, calcium acetylsalicylate, carbamazepine, carbiphene, carprofen, carsalam, chlorobutanol, chlorthenoxazin, choline salicylate, cinchophen, cinmetacin, ciramadol, clidanac, clometacin, clonitazene, clonixin, clopirac, clove, codeine, codeine methyl bromide, codeine phosphate, codeine sulfate, cropropamide, crotethamide, desomorphine, dexoxadrol, dextromoramide, dezocine, diampromide, diclofenac sodium, difenamizole, difenpiramide, diflunisal, dihydrocodeine, dihydrocodeinone enol acetate, dihydromorphine, dihydroxyalutninum acetylsalicylate, dimenoxadol, dimepheptanol, dimethylthiambutene, dioxaphetyl butyrate, dipipanone, diprocetyl, dipyrone, ditazol, droxicam, emorfazone, enfenamic acid, epirizole, eptazocine, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, flufenamic acid, flunoxaprofen, fluoresone, flupirtine, fluproquazone, flurbiprofen, fosfosal, gentisic acid, glafenine, glucametacin, glycol salicylate, guaiazulene, hydrocodone, hydromorphone, hydroxypethidine, ibufenac, ibuprofen, ibuproxam, imidazole salicylate, indomethacin, indoprofen, isofezolac, isoladol, isomethadone, isonixin, isoxepac, isoxicam, ketobemidone, ketoprofen, ketorolac, p-lactophenetide, lefetamine, levorphanol, lofentanil, lonazolac, lomoxicam, loxoprofen, lysine acetylsalicylate, magnesium acetylsalicylate, meclofenamic acid, mefenamic acid, meperidine, meptazinol, mesalamine, metazocine, methadone hydrochloride, methotrimeprazine, metiazinic acid, metofoline, metopon, mofebutazone, mofezolac, morazone, morphine, morphine hydrochloride, morphine sulfate, morpholine salicylate, myrophine, nabumetone, nalbuphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paranyline, parsalmide, pentazocine, perisoxal, phenacetin, phenadoxone, phenazocine, phenazopyridine hydrochloride, phenocoll, phenoperidine, phenopyrazone, phenyl acetylsalicylate, phenylbutazone, phenyl salicylate, phenyramidol, piketoprofen, piminodine, pipebuzone, piperylone, piprofen, pirazolac, piritramide, piroxicam, pranoprofen, proglumetacin, proheptazine, promedol, propacetamol, propiram, propoxyphene, propyphenazone, proquazone, protizinic acid, ramifenazone, remifentanil, rimazolium metilsulfate, salacetamide, salicin, salicylamide, salicylamide o-acetic acid, salicylsulfuric acid, salsalte, salverine, simetride, sodium salicylate, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tolfenamic acid, tolmetin, tramadol, tropesin, viminol, xenbucin, ximoprofen, zaltoprofen and zomepirac. Analgesics are well known in the art. See, for example, The Merck Index, 12th Edition (1996), Therapeutic Category and Biological Activity Index, and the lists provided under “Analgesic”, “Anti-inflammatory” and “Antipyretic”. In an aspect, the recitation of an analgesic inherently encompasses the pharmaceutically acceptable salts thereof.

### Immunostimulants

The term “immunostimulant” is used herein to describe a substance which evokes, increases, and/or prolongs an immune response to an antigen. Immunomodulatory agents modulate the immune system, and, in an aspect, immunostimulants are also referred to as immunomodulatory agents, where it is understood that the desired modulation is to stimulate the immune system. There are two main categories of immunostimulants, specific and non-specific. Specific immunostimulants provide antigenic specificity in immune response, such as vaccines or any antigen, and non-specific immunostimulants act irrespective of antigenic specificity to augment immune response of other antigen or stimulate components of the immune system without antigenic specificity, such as adjuvants and non-specific immunostimulators. Immunostimulants can include, but are not limited to, levamisole, thalidomide, erythema nodosum leprosum, BCG, cytokines such as interleukins or interferons, including recombinant cytokines and interleukin 2 (aldeslukin), 3D-MPL, QS21, CpG ODN 7909, miltefosine, anti-PD-1 or PD-1 targeting drugs, and acid (DCA, a macrophage stimulator), imiquimod and resiquimod (which activate immune cells through the toll-like receptor 7), chlorooxygen compounds such as tetrachlorodecaoxide (TCDO), agonistic CD40 antibodies, soluble CD40L, 4-1BB: 4-1BBL agonists, OX40 agonists, TLR agonists, moieties that deplete regulatory T cells, arabinitol-ceramide, glycerol-ceramide, 6-deoxy and 6-sulfono-myo-insitolceramide, iNKT agonists, and TLR agonists. In an aspect, the recitation of an immunostimulant inherently encompasses the pharmaceutically acceptable salts thereof.

### Immune-Based Product

In an aspect, immune-based products include, but are not limited to, toll-like receptors modulators such as tlr1, tlr2, tlr3, tlr4, tlr5, tlr6, tlr7, tlr8, tlr9, tlr10, tlr11, tlr12, and tlr13; programmed cell death protein 1 (Pd-1) modulators; programmed death-ligand 1 (Pd-L1) modulators; IL-15 agonists; DermaVir; interleukin-7; plaquenil (hydroxychloroquine); proleukin (aldesleukin, IL-2); interferon alfa; interferon alfa-2b; interferon alfa-n3; pegylated interferon alfa; interferon gamma; hydroxyurea; mycophenolate mofetil (MPA) and its ester derivative mycophenolate mofetil (MMF); ribavirin; rintatolimod, polymer polyethyleneimine (PEI); gepon; rintatolimod; IL-12; WF-10; VGV-1; MOR-22; BMS-936559; CYT-107, interleukin-15/Fc fusion protein, normferon, peginterferon alfa-2a, peginterferon alfa-2b, recombinant interleukin-15, RPI-MN, GS-9620, and IR-103. In an aspect, the recitation of an immune-based product inherently encompasses the pharmaceutically acceptable salts thereof.

In an aspect, a disclosed pharmaceutical formulation can be administered to a healthy subject, for example, a subject that does not have an infection and/or is not suspected of having an infection. In an aspect, a disclosed composition can be used with one or more broad-spectrum antibiotic or antibiotic agents.

Disclosed herein is a repository of gut microbiome data generated by one or more methods disclosed here. Disclosed herein is a repository of gut microbiome generated by genera positively associated with ST131 E. coli intestinal carriage. Disclosed herein is a repository of gut microbiome generated by genera positively associated with H30R intestinal carriage. Disclosed herein is a repository of gut microbiome generated by genera positively associated with H30Rx intestinal carriage. Disclosed herein is a repository of gut microbiome generated by genera negatively associated with ST131 E. coli intestinal carriage. Disclosed herein is a repository of gut microbiome generated by genera negatively associated with H30R intestinal carriage. Disclosed herein is a repository of gut microbiome generated by genera negatively associated with H30Rx intestinal carriage.

### C. Methods of Reducing the Risk of Developing an Infection

Disclosed herein is a method of reducing the risk of developing a drug-resistant E. coli infection, the method comprising administering to a subject at risk of developing an E. coli infection a therapeutically effective amount of a composition comprising one or more bacteria belonging to the Alistipes genus and/or Akkermansia genus, wherein following the administering step the composition reduces the intestinal carriage associated with the colonization of drug-resistant E. coli bacteria, thereby reducing the risk of infection.

Disclosed herein is a method of reducing the risk of developing a drug-resistant E. coli infection, the method comprising administering to a subject at risk of developing a drug-resistant E. coli infection a therapeutically effective amount of a composition that modulates the intestinal carriage associated with the colonization of drug-resistant E. coli bacteria, thereby reducing the risk of a drug-resistant E. coli infection.

In an aspect, one or more disclosed E. coli bacteria can comprise sequence type 131 (ST131). In an aspect, one or more disclosed ST131 E. coli bacteria can comprise a H30R subclone. In an aspect, a disclosed H30R subclone can comprise H30R1 or H30Rx. In an aspect, one or more disclosed E. coli bacteria can be multi-drug resistant. In an aspect, one or more disclosed E. coli bacteria are fluoroquinolone resistant. In an aspect, one or more disclosed E. coli bacteria can comprise sequence type 131 (ST131) and can be multi-drug resistant. In an aspect, one or more disclosed ST131 E. coli bacteria can comprise a H30R subclone and can be multi-drug resistant. In an aspect, one or more disclosed H30R subclones can comprise H30R1 or H30Rx and can be multi-drug resistant. In an aspect, one or more disclosed E. coli bacteria can comprise sequence type 131 (ST131) E. coli bacteria and can be fluoroquinolone resistant. In an aspect, one or more disclosed ST131 E. coli bacteria can comprise a H30R subclone and can be fluoroquinolone resistant. In an aspect, one or more disclosed H30R subclones can comprise H30R1 or H30Rx and can be fluoroquinolone resistant.

In an aspect, a disclosed composition can comprise both one or more bacteria belonging to the Alistipes genus and one or more bacteria belonging to the Akkermansia genus. In an aspect, a disclosed composition can comprise one or more bacteria belonging to the Alistipes genus. In an aspect, a disclosed composition can comprise one or more bacteria belonging to an addition genus. In an aspect, a disclosed composition can comprise one or more bacterial belonging to the Alistipes genus and one or more bacteria belonging to the Akkermansia genus. In an aspect, a disclosed composition can comprise one or more bacteria belonging to the Alistipes genus. In an aspect, the Alistipes genus can comprise Alistipes finegoldii, Alistipes putredinis, Alistipes onderdonkii, Alistipes shahii, Alistipes indistinctus, Alistipes senegalensis, Alistipes timonensis, Alistipes obesi, Alistipes ihumii, Alistipes inops, Alistipes megaguti, Alistipes provencensis, Alistipes massiliensis, any combination thereof. In an aspect, a disclosed composition can comprise one or more bacterial species belonging to the Verrucomicrobia phylum. In an aspect, a disclosed composition can comprise one or more bacterial belonging to the Alistipes genus and one or more bacteria belonging to the Akkermansia genus.

In an aspect, a disclosed composition can comprise one or more agents that can reduce the intestinal carriage associated with the colonization of drug-resistant E. coli bacteria, thereby reducing the risk of an infection. In an aspect, a disclosed composition can comprise one or more agents that can reduce the proportional abundance of one or more bacteria associated with the carriage and/or persistence of drug-resistant E. coli bacteria, thereby reducing the degree and/or severity of colonization by a drug-resistance E. coli. In an aspect, a disclosed composition can comprise one or more agents that can inhibit proliferation of Asaccharobacter, Bifidobacterium, Collinsella, Eggerthella, Gardnerella, Gordonibacter, or any combination thereof in the gut microbiome of the subject. In an aspect, a disclosed composition can comprise one or more agents that can increase proliferation of Alistipes and/or Akkermansia.

In an aspect, a disclosed composition can comprise (i) one or more agents that can reduce the intestinal carriage associated with the colonization of drug-resistant E. coli bacteria, (ii) one or more agents that can reduce the proportional abundance of one or more bacteria associated with the carriage and/or persistence of drug-resistant E. coli bacteria, (iii) one or more agents that can inhibit proliferation of Asaccharobacter, Bifidobacterium, Collinsella, Eggerthella, Gardnerella, Gordonibacter, or any combination thereof in the gut microbiome of the subject, (iv) one or more agents that can increase proliferation of Alistipes and/or Akkermansia, or (v) any combination thereof.

In an aspect, relative species abundance is a component of biodiversity. In an aspect, relative species abundance is a measure of how common or rare a species is relative to other species in a defined location or community. In an aspect, relative species abundance is a measure of how common or rare a species is relative to other species in a subject's gut microbiome. In an aspect, relative species abundance is a measure of how common or rare a species is relative to other species in a subject's gut microbiome prior to a disclosed administering step. In an aspect, relative species abundance is a measure of how common or rare a species is relative to other species in a subject's gut microbiome following a disclosed administering step. In an aspect, relative abundance is the percent composition of an organism of a particular kind relative to the total number of organisms in the area. In an aspect, relative abundance is the percent composition of an organism of a particular kind relative to the total number of organisms in a subject's gut microbiome. In an aspect, relative abundance is the percent composition of an organism of a particular kind relative to the total number of organisms in a subject's gut microbiome prior to a disclosed administering step. In an aspect, relative abundance is the percent composition of an organism of a particular kind relative to the total number of organisms in a subject's gut microbiome following a disclosed administering step.

In an aspect, abundance can be the number of individuals of a given species in a region. In an aspect, incidence can be the number of samples containing at least one of a given species in a census. In an aspect, sampling scope can be the area, interval of time, and taxonomic grouping over which sampling takes place. In an aspect, and in the context of community sampling, spatial heterogeneity can be variation in species capture probabilities across space. In an aspect, spatial heterogeneity can be due to habitat variation, intraspecific clumping, interspecific association, other factors, or any combination thereof. In an aspect, species abundance distribution can be the number of species found in each interval of abundance in a community.

In an aspect, species accumulation curve can be a plot of the total number of species observed in a census against some measure of cumulative sampling effort. In an aspect, species accumulation can be any of a number of measures concerning the number of species in an area and/or the distribution of their abundances. In an aspect, species richness can be the number of species present in a region.

In an aspect, and in the context of community sampling, temporal heterogeneity can be variation in species capture probabilities over time. In an aspect, temporal heterogeneity can arise from temporal environmental variation, migration, speciation or other factors. In an aspect, bacterial diversity can comprise two components: (i) species richness (i.e., the number of species in the community), and (ii) evenness (i.e., the fact that some species in the community are common and others are rare). In an aspect, an important component of most diversity indices is a value expressing the relative proportion of the species in the community (pi). In an aspect, this value can be calculated as the absolute (raw) abundance or dominance of the species (i) in the community, divided by the sum of all species abundances/dominances in that community. In an aspect, the sum of relative abundances for all species in the community equals to unity.

In an aspect of a disclosed method of reducing the risk of developing a drug-resistant E. coli infection, prior to the administering step the gut microbiome of the subject can comprise a higher proportional abundance of one or more bacteria belonging to the Actinobacteria phylum than that of a control subject. In an aspect, Actinobacteria phylum can comprise Asaccharobacter, Bifidobacterium, Collinsella, Eggerthella, Gardnerella, Gordonibacter, or any combination thereof. In an aspect of a disclosed method of reducing the risk of developing a drug-resistant E. coli infection, prior to the administering step the gut microbiome of the subject can comprise a higher proportional abundance of Collinsella than that of a control subject. In an aspect of a disclosed method of reducing the risk of developing a drug-resistant E. coli infection, prior to the administering step the gut microbiome of the subject can comprise a higher proportional abundance of Gardnerella and/or Gordonibacter than that of a control subject. In an aspect of a disclosed method of reducing the risk of developing a drug-resistant E. coli infection, prior to the administering step the gut microbiome of the subject can comprise a higher proportional abundance of one or more bacteria belonging to the Firmicutes phylum than that of a control subject. In an aspect, Firmicutes phylum can comprise Enterococcus, Lactobacillus, Streptococcus, Weissella, or any combination thereof. In an aspect of a disclosed method of reducing the risk of developing a drug-resistant E. coli infection, prior to the administering step the gut microbiome of the subject can comprise a higher proportional abundance of Lactobacillus and/or Streptococcus than that of a control subject.

In an aspect of a disclosed method of reducing the risk of developing a drug-resistant E. coli infection, prior to the administering step the gut microbiome of the subject can comprise a lower proportional abundance of one or more bacteria belonging to the Bacteroidetes phylum than that of a control subject. In an aspect, Bacteroidetes phylum can comprise Alistipes and/or Bacteroides. In an aspect, Bacteroides can comprise B. dorei, B. uniformis, B. eggerthii, B. stercoris, B. fragilis, B. caccae, B. intestinalis, B. ovatus, B. xylanisolvens, B. splanchnicus, or any combination thereof. In an aspect, Alistipes can comprise Alistipes finegoldii, Alistipes putredinis, Alistipes onderdonkii, Alistipes shahii, Alistipes indistinctus, Alistipes senegalensis, Alistipes timonensis, Alistipes obesi, Alistipes ihumii, Alistipes inops, Alistipes megaguti, Alistipes provencensis, Alistipes massiliensis, any combination thereof.

In an aspect, prior to the administering step the gut microbiome of the subject can comprise a lower proportional abundance of Bacteroides than that of a control subject. In an aspect, prior to the administering step the gut microbiome of the subject can comprise a lower proportional abundance of Alistipes than that of a control subject. In an aspect, prior to the administering step the gut microbiome of the subject can comprise a lower proportional abundance of one or more bacteria belonging to Verrucomicrobia phylum than that of a control subject. In an aspect, Verrucomichrobia phylum can comprise family Akkermansiaceae and family Verrucomicrobiaceae. In an aspect, Akkermansiaceae can comprise Akkermansia. In an aspect of a disclosed method of reducing the risk of developing a drug-resistant E. coli infection, wherein prior to the administering step the gut microbiome of the subject can comprise a lower proportional abundance of Akkermansia than that of a control subject.

In an aspect of a disclosed method of reducing the risk of developing a drug-resistant E. coli infection, wherein following the administering step, the proportional abundance of one or more E. coli sequence type 131 (ST131) bacteria in the gut microbiome of the subject is decreased. In an aspect of a disclosed method of reducing the risk of developing a drug-resistant E. coli infection, wherein, following the administering step, the proportional abundance of ST131-H30R1 in the gut microbiome of the subject is decreased. In an aspect of a disclosed method of reducing the risk of developing a drug-resistant E. coli infection, wherein, following the administering step, the proportional abundance of ST131-H30Rx in the gut microbiome of the subject is decreased. In an aspect of a disclosed method of reducing the risk of developing a drug-resistant E. coli infection, wherein, following the administering step, the proportional abundance of ST131-H30R1 and ST131-H30Rx in the gut microbiome of the subject is decreased. In an aspect of a disclosed method of reducing the risk of developing a drug-resistant E. coli infection, wherein, following the administering step, the level of and/or the amount of ST131-H30R1 and/or the level of and/or the amount of ST131-H30Rx in the gut microbiome of the subject is decreased from the level and/or the amount prior to the administering step.

In an aspect, a disclosed method can further comprise administering to the subject one or more bacteriophages capable of killing E. coli. In an aspect, a disclosed E. coli-killing bacteriophage can comprise BP539, BP700, BP753, BP814, BP953, BP954, BP970, BP1002, BP1151, BP1155, BP1168, BP1176, BP1197, BP1226, BP1229, or any combination thereof. In an aspect, a disclosed E. coli-killing bacteriophage can comprise MV36.2, JJ2050.2, C3, C19T, JJ6.1, or any combination thereof.

In an aspect of a disclosed method of reducing the risk of developing a drug-resistant E. coli infection, wherein, following the administering step, the proportional abundance of one or more bacteria belonging to Actinobacteria phylum in the gut microbiome of the subject is decreased. In an aspect of a disclosed method of reducing the risk of developing a drug-resistant E. coli infection, wherein, following the administering step, the proportional abundance of Asaccharobacter, Bifidobacterium, Collinsella, Eggerthella, Gardnerella, Gordonibacter, or any combination thereof in the gut microbiome of the subject is decreased. In an aspect of a disclosed method of reducing the risk of developing a drug-resistant E. coli infection, wherein, following the administering step, the proportional abundance of Collinsella in the gut microbiome of the subject is decreased. In an aspect of a disclosed method of reducing the risk of developing a drug-resistant E. coli infection, wherein, following the administering step, the proportional abundance of Gardnerella and/or Gordonibacter in the gut microbiome of the subject is decreased. In an aspect of a disclosed method of reducing the risk of developing a drug-resistant E. coli infection, wherein, following the administering step, the proportional abundance of one or more bacteria belonging to Firmicutes phylum in the gut microbiome of the subject is decreased.

In an aspect of a disclosed method of reducing the risk of developing a drug-resistant E. coli infection, wherein, following the administering step, the proportional abundance of Enterococcus, Lactobacillus, Streptococcus, Weissella, or any combination thereof in the gut microbiome of the subject is decreased.

In an aspect of a disclosed method of reducing the risk of developing a drug-resistant E. coli infection, wherein, following the administering step, the proportional abundance of Lactobacillus and/or Streptococcus in the gut microbiome of the subject is decreased.

In an aspect of a disclosed method of reducing the risk of developing a drug-resistant E. coli infection, wherein, following the administering step, the level of and/or the amount of ST131-H30R1 and/or ST131-H30Rx in the gut microbiome of the subject is decreased from the level and/or the amount prior to the administering step. In an aspect of a disclosed method of reducing the risk of developing a drug-resistant E. coli infection, wherein, following the administering step, the level of and/or the amount of one or more bacteria belonging to the Actinobacteria phylum in the gut microbiome of the subject is decreased from the level and/or the amount prior to the administering step.

In an aspect of a disclosed method of reducing the risk of developing a drug-resistant E. coli infection, wherein, following the administering step, the level of and/or the amount of one or more bacteria belonging to the Actinobacteria phylum in the gut microbiome of the subject is decreased from the level and/or the amount prior to the administering step. In an aspect of a disclosed method of reducing the risk of developing a drug-resistant E. coli infection, wherein, following the administering step, the level of and/or the amount of Asaccharobacter, Bifidobacterium, Collinsella, Eggerthella, Gardnerella, Gordonibacter, or any combination thereof in the gut microbiome of the subject is decreased from the level and/or the amount prior to the administering step. In an aspect of a disclosed method of reducing the risk of developing a drug-resistant E. coli infection, wherein, following the administering step, the level of and/or the amount Collinsella in the gut microbiome of the subject is decreased from the level and/or the amount prior to the administering step.

In an aspect of a disclosed method of reducing the risk of developing a drug-resistant E. coli infection, wherein, following the administering step, the level of and/or the amount of Gardnerella and/or Gordonibacter in the gut microbiome of the subject is decreased from the level and/or the amount prior to the administering step.

In an aspect of a disclosed method of reducing the risk of developing a drug-resistant E. coli infection, wherein, following the administering step, the level of and/or the amount of one or more bacteria belonging to the Firmicutes phylum in the gut microbiome of the subject is decreased from the level and/or the amount prior to the administering step. In an aspect of a disclosed method of reducing the risk of developing a drug-resistant E. coli infection, wherein, following the administering step, the level of and/or the amount of Enterococcus, Lactobacillus, Streptococcus, Weissella, or any combination thereof in the gut microbiome of the subject is decreased from the level and/or the amount prior to the administering step. In an aspect of a disclosed method of reducing the risk of developing a drug-resistant E. coli infection, wherein, following the administering step, the level of and/or the amount of Lactobacillus and/or Streptococcus in the gut microbiome of the subject is decreased from the level and/or the amount prior to the administering step.

In an aspect, gastrointestinal administration can comprise oral, nasogastric, or rectal administration. In an aspect, a disclosed composition can comprise a liquid, a suspension, a dried powder, a tablet, a capsule, or a food product.

In an aspect, a disclosed method of reducing the risk of developing a drug-resistant E. coli infection can further comprise administering to the subject an antibiotic, an anti-toxin antibody, an herbal remedy, a probiotic bacteria, a probiotic yeast, or any combination thereof.

In an aspect, a disclosed method of reducing the risk of developing a drug-resistant E. coli infection can further comprise administering to the subject one or more antibiotics, anti-toxin antibodies, herbal remedies, probiotic bacteria, probiotic yeast, or any combination thereof.

In an aspect, a disclosed method of reducing the risk of developing a drug-resistant E. coli infection can further comprise administering to the subject antibiotic, an anti-toxin antibody, an herbal remedy, a probiotic bacteria, a probiotic yeast, or any combination thereof.

In an aspect of a disclosed method of reducing the risk of developing a drug-resistant E. coli infection, the therapeutically effective amount can ameliorate at least one symptom of an E. coli infection. In an aspect, a disclosed symptom of an E. coli infection can comprise abdominal tenderness, abdominal pain, abdominal cramping, sepsis, endocarditis, meningitis, headache, stiff neck, confusion, back pain, pneumonia, fever, chills, diarrhea, urinary tract infection, endocarditis, elevated white blood cell count, and decreased serum albumin.

In an aspect, a disclosed therapeutically effective amount can inhibit proliferation of one or more E. coli bacteria in the gastrointestinal tract of the subject. In an aspect, a disclosed therapeutically effective amount can inhibit proliferation of one or more E. coli bacteria in the gut microbiome of the subject.

In an aspect, a disclosed therapeutically effective amount can inhibit proliferation of E. coli bacteria can comprise sequence type 131 (ST131) in the gastrointestinal tract of the subject. In an aspect, a disclosed therapeutically effective amount can inhibit proliferation of E. coli bacteria can comprise sequence type 131 (ST131) in the gut microbiome of the subject.

In an aspect, a disclosed therapeutically effective amount can inhibit proliferation of H30R in the gastrointestinal tract of the subject. In an aspect, a disclosed therapeutically effective amount can inhibit proliferation of H30R in the gut microbiome of the subject.

In an aspect, a disclosed therapeutically effective amount can inhibit proliferation of H30Rx in the gastrointestinal tract of the subject. In an aspect, a disclosed therapeutically effective amount can inhibit proliferation of H30Rx in the gut microbiome of the subject.

In an aspect, a disclosed therapeutically effective amount can inhibit proliferation of one or more bacteria belonging to Actinobacteria phylum in the gastrointestinal tract of the subject. In an aspect, a disclosed therapeutically effective amount can inhibit proliferation of one or more bacteria belonging to Actinobacteria phylum in the gut microbiome of the subject.

In an aspect, a disclosed therapeutically effective amount can inhibit proliferation of Asaccharobacter, Bifidobacterium, Collinsella, Eggerthella, Gardnerella, Gordonibacter, or any combination thereof in the gastrointestinal tract of the subject. In an aspect, a disclosed therapeutically effective amount can inhibit proliferation of Asaccharobacter, Bifidobacterium, Collinsella, Eggerthella, Gardnerella, Gordonibacter, or any combination thereof in the gut microbiome of the subject.

In an aspect, a disclosed therapeutically effective amount can inhibit proliferation of Collinsella in the gastrointestinal tract of the subject. In an aspect, a disclosed therapeutically effective amount can inhibit proliferation of Collinsella in the gut microbiome of the subject.

In an aspect, a disclosed therapeutically effective amount can inhibit proliferation of Gardnerella and/or Gordonibacter in the gastrointestinal tract of the subject. In an aspect, a disclosed therapeutically effective amount can inhibit proliferation of Gardnerella and/or Gordonibacter in the gut microbiome of the subject.

In an aspect, a disclosed therapeutically effective amount can inhibit proliferation of one or more bacteria belonging to Firmicutes phylum in the gastrointestinal tract of the subject. In an aspect, a disclosed therapeutically effective amount can inhibit proliferation of one or more bacteria belonging to Firmicutes phylum in the gut microbiome of the subject.

In an aspect, a disclosed therapeutically effective amount can inhibit proliferation of Enterococcus, Lactobacillus, Streptococcus, Weissella, or any combination thereof in the gastrointestinal tract of the subject. In an aspect, a disclosed therapeutically effective amount can inhibit proliferation of Enterococcus, Lactobacillus, Streptococcus, Weissella, or any combination thereof in the gut microbiome of the subject.

In an aspect, a disclosed therapeutically effective amount can inhibit proliferation of Lactobacillus and/or Streptococcus in the gastrointestinal tract of the subject. In an aspect, a disclosed therapeutically effective amount can inhibit proliferation of Lactobacillus and/or Streptococcus in the gut microbiome of the subject.

In an aspect of a disclosed method of reducing the risk of developing a drug-resistant E. coli infection, wherein following the administering step, the subject can be now susceptible and/or more susceptible to fluoroquinolone. In an aspect of a disclosed method of reducing the risk of developing a drug-resistant E. coli infection, wherein following the administering step, the subject can be now susceptible and/or more susceptible to antibiotic treatment. In an aspect of a disclosed method of reducing the risk of developing a drug-resistant E. coli infection, wherein following the administering step, the subject can be now susceptible and/or more susceptible to a treatment regimen comprising one or more antibiotic agents.

In an aspect, a disclosed method of reducing the risk of developing a drug-resistant E. coli infection can further comprise measuring the prevalence and/or proportional abundance of one or more Escherichia coli (E. coli) bacteria. In an aspect, E. coli bacteria can comprise sequence type 131 (ST131). In an aspect, ST131 E. coli bacteria can comprise a H30R subclone. In an aspect, a disclosed H30R subclone can comprise H30R1 or H30Rx. In an aspect, a disclosed E. coli bacteria can comprise sequence type 131 (ST131) and can be multi-drug resistant. In an aspect, a disclosed ST131 E. coli bacteria can comprise a H30R subclone and can be multi-drug resistant. In an aspect, a disclosed H30R subclone can comprise H30R1 or H30Rx and can be multi-drug resistant. In an aspect, a disclosed E. coli bacteria can be multi-drug resistant. In an aspect, a disclosed E. coli bacteria can be fluoroquinolone resistant. In an aspect, a disclosed E. coli bacteria can comprise sequence type 131 (ST131) E. coli bacteria and can be fluoroquinolone resistant. In an aspect, a disclosed ST131 E. coli bacteria can comprise a H30R subclone and can be fluoroquinolone resistant. In an aspect, a disclosed H30R subclone can comprise H30R1 or H30Rx and can be fluoroquinolone resistant.

In an aspect, measuring the prevalence and/or proportional abundance of one or more bacteria in the subject's gut microbiome can inform a clinician's treatment decisions.

In an aspect, a disclosed method of reducing the risk of developing a drug-resistant E. coli infection can further comprise obtaining one or more biological samples from the subject.

In an aspect, a disclosed method of reducing the risk of developing a drug-resistant E. coli infection can further comprise repeating the obtaining of one or more biological samples from the subject.

In an aspect, the repeating of the obtaining step can occur at different times. In an aspect, the first obtaining step can be performed prior to administering any treatment to the subject. In an aspect, the second obtaining step can be performed after administering any treatment to the subject. In an aspect, subsequent obtaining steps can be performed after administering any treatment to the subject.

In an aspect, a disclosed method of reducing the risk of developing a drug-resistant E. coli infection can further comprise comparing the prevalence and/or proportional abundance of one or more bacteria in the subject's gut microbiome to one or more standard levels. In an aspect, a disclosed method of reducing the risk of developing a drug-resistant E. coli infection can further comprise comparing the prevalence and/or proportional abundance of one or more bacteria in the subject's gut microbiome to one or more control levels.

In an aspect, a disclosed method of reducing the risk of developing a drug-resistant E. coli infection can further comprise comparing the prevalence and/or proportional abundance of the one or more bacteria in the subject's gut microbiome to one or more standard levels, and if the prevalence and/or proportional abundance of the one or more bacteria in the subject's gut microbiome is similar to that of one or more subjects having demonstrated responsiveness to treatment, then characterizing the subject as likely to be responsive to treatment.

In an aspect, a disclosed method of reducing the risk of developing a drug-resistant E. coli infection can further comprise comparing the prevalence and/or proportional abundance of the one or more bacteria in the subject's gut microbiome to one or more standard levels, and if the prevalence and/or proportional abundance of the one or more bacteria in the subject's gut microbiome is dissimilar to that of one or more subjects having demonstrated responsiveness to treatment, then characterizing the subject as likely to be non-responsive to treatment.

In an aspect, a disclosed method of reducing the risk of developing a drug-resistant E. coli infection can further comprise comparing the prevalence and/or proportional abundance of the one or more bacteria in the subject's gut microbiome to one or more standard levels, and if the prevalence and/or proportional abundance of the one or more bacteria in the subject's gut microbiome is similar to that of one or more subjects having demonstrated non-responsiveness to treatment, then characterizing the subject as likely to be non-responsive to treatment.

In an aspect, a disclosed method of reducing the risk of developing a drug-resistant E. coli infection can further comprise comparing the prevalence and/or proportional abundance of the one or more bacteria in the subject's gut microbiome to one or more standard levels, and if the prevalence and/or proportional abundance of the one or more bacteria in the subject's gut microbiome is dissimilar to that of one or more subjects having demonstrated non-responsiveness to treatment, then characterizing the subject as likely to be responsive to treatment.

In an aspect, a disclosed method of reducing the risk of developing a drug-resistant E. coli infection can further comprise generating a standard level or a standard range for prevalence and/or proportional abundance of one or more bacteria in a gut microbiome. In an aspect, a disclosed method of reducing the risk of developing a drug-resistant E. coli infection can further comprise generating a standard level or a standard range for prevalence and/or proportional abundance of one or more bacteria in a gut microbiome, wherein the standard level or the standard range represents a likelihood of non-responsiveness to treatment. In an aspect, a disclosed method of reducing the risk of developing a drug-resistant E. coli infection can further comprise generating a standard level or a standard range for prevalence and/or proportional abundance of one or more bacteria in a gut microbiome, wherein the standard level or the standard range represents a likelihood of responsiveness to treatment. In an aspect, a disclosed method of reducing the risk of developing a drug-resistant E. coli infection can further comprise treating the subject.

In an aspect, treating the subject can comprise administering a composition comprising (i) one or more agents that can reduce the intestinal carriage associated with the colonization of drug-resistant E. coli bacteria, (ii) one or more agents that can reduce the proportional abundance of one or more bacteria associated with the carriage and/or persistence of drug-resistant E. coli bacteria, (iii) one or more agents that can inhibit proliferation of Asaccharobacter, Bifidobacterium, Collinsella, Eggerthella, Gardnerella, Gordonibacter, or any combination thereof, (iv) one or more agents that can increase proliferation of Alistipes and/or Akkermansia.

In an aspect, a disclosed method of reducing the risk of developing a drug-resistant E. coli infection can further comprise comprising administering to the subject a therapeutically effective amount of one or more therapeutic agent. In an aspect, therapeutic agents can comprise any therapeutic agent disclosed herein. In an aspect, treating can comprise administering to the subject a therapeutically effective amount of one or more disclosed compositions. In an aspect, treating can comprise administering to the subject a therapeutically effective amount of one or more disclosed pharmaceutical formulations.

In an aspect, a disclosed sample can comprise a fecal sample. In an aspect, a disclosed fecal sample can be obtained using a fecal swab. In an aspect, the prevalence and/or the proportional abundance of the one or more bacterial in a sample can be generated by sequencing the 16S rRNA gene V3-V4 region. In an aspect, sequencing the 16S rRNA gene V3-V4 region can be performed according to the method described in as described in Fadrosh D W, et al. (2014) Microbiome. 2 (1): 6 by using MiSeq Reagent Kit v3 (600-cycle) (Illumina Inc., San Diego, CA). In an aspect, one or more negative extraction controls (NECs) can be included with each batch of extraction and can be sequenced to assess for cross contamination. In an aspect, one or more no-template controls (NTCs) and one or more positive-template controls (PTCs) can be included to assess for cross-contamination and to verify PCR performance.

In an aspect, a disclosed method of reducing the risk of developing a drug-resistant E. coli infection can further comprise repeating the treating step one or more times. In an aspect, a disclosed method of reducing the risk of developing a drug-resistant E. coli infection can further comprise repeating the treating step until the subject experiences an alleviation and/or a diminishment of one or more symptoms. In an aspect, the one or more symptoms can be associated with and/or due to the presence of one or more disclosed bacteria in the subject's gut microbiome. In an aspect, the presence of the one or more bacteria in the subject's gut microbiome can interfere with treating the subject for one or more diseases, infections, and/or disorder.

In an aspect, the presence of the one or more ST131 E. coli bacteria in the subject's gut microbiome can interfere with treating the subject for one or more diseases, infections, and/or disorder. In an aspect, the presence of the H30R1 E. coli bacteria and/or H30Rx E. coli bacteria in the subject's gut microbiome can interfere with treating the subject for one or more diseases, infections, and/or disorder. In an aspect, interfering can comprise disrupting and/or diminishing the efficacy of one or more treatments. In an aspect, interfering can comprise rendering the subject resistant to one or more antibiotics. In an aspect, interfering can comprise rendering the subject resistant to fluoroquinolone. In an aspect, the one or more E. coli bacteria can comprise sequence type 131 (ST131) and can be multi-drug resistant. In an aspect, the one or more ST131 E. coli bacteria can comprise a H30R subclone and can be multi-drug resistant. In an aspect, the one or more H30R subclones can comprise H30R1 or H30Rx and is multi-drug resistant. In an aspect, the one or more E. coli bacteria can be multi-drug resistant. In an aspect, the one or more E. coli bacteria can be fluoroquinolone resistant. In an aspect, the one or more E. coli bacteria can comprise sequence type 131 (ST131) E. coli bacteria and can be fluoroquinolone resistant. In an aspect, the one or more ST131 E. coli bacteria can comprise a H30R subclone and can be fluoroquinolone resistant. In an aspect, the one or more H30R subclones can comprise H30R1 or H30Rx and is fluoroquinolone resistant.

In an aspect, the one or more bacteria can comprise bacteria in the Actinobacter phylum, the Bacteroidetes phylum, the Firmicutes phylum, the Verruncomicrobia phylum, the Proteobacteria phylum, or any combination thereof. In an aspect, a higher proportional abundance of Actinobacteria can indicate that the subject is not likely to be responsive to antibiotic treatment. In an aspect, a lower proportional abundance of Actinobacteria can indicate that the subject is likely to be responsive to antibiotic treatment. In an aspect, a higher proportional abundance of Collinsella can indicate that the subject is not likely to be responsive to antibiotic treatment. In an aspect, a lower proportional abundance of Collinsella can indicate that the subject is likely to be responsive to antibiotic treatment. In an aspect, a lower proportional abundance of Verrucomicrobia can indicate that the subject is not likely to be responsive to antibiotic treatment. In an aspect, a higher proportional abundance of Verrucomicrobia can indicate that the subject is likely to be responsive to antibiotic treatment. In an aspect, a higher proportional abundance of Alistipes can indicate that the subject is likely to be responsive to antibiotic treatment. In an aspect, a lower proportional abundance of Alistipes can indicate that the subject is not likely to be responsive to antibiotic treatment. In an aspect, a higher proportional abundance of Akkermansia can indicate that the subject is likely to be responsive to antibiotic treatment. In an aspect, a lower proportional abundance of Akkermansia can indicate that the subject is not likely to be responsive to antibiotic treatment.

In an aspect, a higher proportional abundance of Actinobacteria can indicate a likelihood that the subject will be non-responsive to antibiotic treatment. In an aspect, a lower proportional abundance of Actinobacteria can indicate a likelihood that the subject will be responsive to antibiotic treatment. In an aspect, a higher proportional abundance of Collinsella can indicate a likelihood that the subject will be non-responsive to antibiotic treatment. In an aspect, a lower proportional abundance of Collinsella can indicate a likelihood that the subject will be responsive to antibiotic treatment. In an aspect, a lower proportional abundance of Verrucomicrobia can indicate a likelihood that the subject will be non-responsive to antibiotic treatment. In an aspect, a higher proportional abundance of Verrucomicrobia can indicate a likelihood that the subject will be responsive to antibiotic treatment. In an aspect, a higher proportional abundance of Alistipes can indicate a likelihood that the subject will be responsive to antibiotic treatment. In an aspect, a lower proportional abundance of Alistipes can indicate a likelihood that the subject will be non-responsive to antibiotic treatment. In an aspect, a higher proportional abundance of Akkermansia can indicate a likelihood that the subject will be responsive to antibiotic treatment. In an aspect, a lower proportional abundance of Akkermansia can indicate a likelihood that the subject will be non-responsive to antibiotic treatment.

In an aspect of a disclosed method of reducing the risk of developing a drug-resistant E. coli infection, a subject is likely to be responsive to antibiotic treatment when there is (i) a lower proportional abundance of Actinobacteria; (ii) a lower proportional abundance of Collinsella; (iii) a higher proportional abundance of Verrucomicrobia; (iv) a higher proportional abundance of Alistipes; (v) a higher proportional abundance of Akkermansia; or (vi) any combination thereof. In an aspect of a disclosed method of reducing the risk of developing a drug-resistant E. coli infection, a subject is not likely to be responsive to antibiotic treatment when there is (i) a higher proportional abundance of Actinobacteria; (ii) a higher proportional abundance of Collinsella; (iii) a lower proportional abundance of Verrucomicrobia; (iv) a lower proportional abundance of Alistipes; (v) a lower proportional abundance of Akkermansia; or (vi) any combination thereof.

In an aspect, a disclosed method of reducing the risk of developing a drug-resistant E. coli infection can further comprise further comprising measuring the prevalence and/or proportional abundance of one or more E. coli bacteria sequence type 131 (ST131). In an aspect, the one or more ST131 E. coli bacteria can comprise a H30R subclone and is fluoroquinolone resistant. In an aspect, the H30R subclone can comprise H30R1 or H30Rx and can be fluoroquinolone resistant.

In an aspect, a disclosed method of reducing the risk of developing a drug-resistant E. coli infection can further comprise administering to the subject one or more compositions that modulate the intestinal carriage and persistence of one or more ST131 E. coli bacteria. In an aspect, a disclosed method of reducing the risk of developing a drug-resistant E. coli infection can further comprise administering to the subject one or more compositions that modulate the intestinal carriage and persistence of H30R1 and/or H30Rx. In an aspect, a disclosed method of reducing the risk of developing a drug-resistant E. coli infection can further comprise administering to the subject one or more compositions that decrease and/or disrupt the intestinal carriage and persistence of one or more ST131 E. coli bacteria. In an aspect, a disclosed method of reducing the risk of developing a drug-resistant E. coli infection can further comprise administering to the subject one or more compositions that decrease and/or disrupt the intestinal carriage and persistence of H30R1 and/or H30Rx. In an aspect, a disclosed method of reducing the risk of developing a drug-resistant E. coli infection can further comprise administering to the subject one or more compositions that inhibit proliferation of H30R1 and/or H30Rx in the subject's gut microbiome and/or gastrointestinal tract.

In an aspect, a disclosed method of reducing the risk of developing a drug-resistant E. coli infection can further comprise administering to the subject one or more compositions that modulate the intestinal carriage and persistence of one or more bacteria. In an aspect, a disclosed method of reducing the risk of developing a drug-resistant E. coli infection can further comprise administering to the subject one or more compositions that decrease and/or disrupt the intestinal carriage and persistence of one or more bacteria. In an aspect, a disclosed method of reducing the risk of developing a drug-resistant E. coli infection can further comprise administering to the subject one or more compositions that inhibit proliferation of one or more bacteria in the subject's gut microbiome and/or gastrointestinal tract. In an aspect, a disclosed method of reducing the risk of developing a drug-resistant E. coli infection can further comprise administering to the subject one or more compositions that inhibit proliferation of one or more bacteria belonging to the Actinobacteria phylum in the subject's gut and/or gastrointestinal tract.

In an aspect, a disclosed method of reducing the risk of developing a drug-resistant E. coli infection can further comprise administering to the subject one or more compositions that inhibit proliferation of Asaccharobacter, Bifidobacterium, Collinsella, Eggerthella, Gardnerella, Gordonibacter, or any combination thereof in the subject's gut and/or gastrointestinal tract.

In an aspect, a disclosed method of reducing the risk of developing a drug-resistant E. coli infection can further comprise administering to the subject one or more compositions that inhibit proliferation of Collinsella in the subject's gut and/or gastrointestinal tract. In an aspect, a disclosed method of reducing the risk of developing a drug-resistant E. coli infection can further comprise administering to the subject one or more compositions that inhibit proliferation of Gardnerella and/or Gordonibacter in the subject's gut and/or gastrointestinal tract. In an aspect, a disclosed method of reducing the risk of developing a drug-resistant E. coli infection can further comprise administering to the subject one or more compositions that inhibit proliferation of one or more bacteria belonging to the Firmicutes phylum in the subject's gut and/or gastrointestinal tract. In an aspect, a disclosed method of reducing the risk of developing a drug-resistant E. coli infection can further comprise administering to the subject one or more compositions that inhibit proliferation of Enterococcus, Lactobacillus, Streptococcus, Weissella, or any combination thereof in the subject's gut and/or gastrointestinal tract. In an aspect, a disclosed method of reducing the risk of developing a drug-resistant E. coli infection can further comprise administering to the subject one or more compositions that inhibit proliferation of Lactobacillus and/or Streptococcus in the subject's gut and/or gastrointestinal tract.

In an aspect, a disclosed method of reducing the risk of developing a drug-resistant E. coli infection can further comprise administering to the subject one or more compositions that decrease and/or disrupt the intestinal carriage and persistence of one or more bacteria. In an aspect, a disclosed method of reducing the risk of developing a drug-resistant E. coli infection can further comprise administering to the subject one or more compositions that increase and/or stimulate proliferation of one or more bacteria in the subject's gut microbiome and/or gastrointestinal tract. In an aspect, a disclosed method of reducing the risk of developing a drug-resistant E. coli infection can further comprise administering to the subject one or more compositions that increase and/or stimulate proliferation of one or more bacteria belonging to the Bacteroidetes phylum in the subject's gut microbiome and/or gastrointestinal tract. In an aspect, a disclosed method of reducing the risk of developing a drug-resistant E. coli infection can further comprise administering to the subject one or more compositions that increase and/or stimulate proliferation of one or more bacteria belonging to the Verrucomicrobia phylum in the subject's gut microbiome and/or gastrointestinal tract. In an aspect, a disclosed method of reducing the risk of developing a drug-resistant E. coli infection can further comprise administering to the subject one or more compositions that increase and/or stimulate proliferation of Alistipes and/or Akkermansia the subject's gut microbiome and/or gastrointestinal tract. In an aspect, a lower proportional abundance of Verrucomicrobia can indicate that the subject is not likely to be responsive to antibiotic treatment. In an aspect, a higher proportional abundance of Verrucomicrobia can indicate that the subject is likely to be responsive to antibiotic treatment. In an aspect, a higher proportional abundance of Alistipes can indicate that the subject is likely to be responsive to antibiotic treatment. In an aspect, a lower proportional abundance of Alistipes can inhibit that the subject is not likely to be responsive to antibiotic treatment. In an aspect, a higher proportional abundance of Akkermansia can inhibit that the subject is likely to be responsive to antibiotic treatment. In an aspect, a lower proportional abundance of Akkermansia can inhibit that the subject is not likely to be responsive to antibiotic treatment.

In an aspect of a disclosed method of reducing the risk of developing a pathogenic E. coli infection, a subject at risk can be any subject. In an aspect, an at-risk subject can comprise an adult, a young adult, a child, a toddler, or an infant. In an aspect, an at-risk subject can have cancer. In an aspect, an at-risk subject can be receiving chemotherapy. In an aspect, an at-risk subject can be receiving immunosuppressive therapy. In an aspect, an at-risk subject that does not have an infection and/or is not suspected of having an infection. In an aspect, following administration, the colonization of a pathogenic E. coli in the subject's intestinal tract and/or gut can be inhibited and/or prevented. In an aspect, a disclosed method of reducing the risk of developing a pathogenic bacterial and/or ST131 E. coli (i.e., H30R1 and/or H30Rx) infection can further comprise promoting respiratory health in a subject. In an aspect, a disclosed biotherapeutic can inhibit and/or prevent the growth of one or more E. coli in one or more parts of the subject's respiratory system. In an aspect, a disclosed method of reducing the risk of developing a E. coli infection can further comprise characterizing the microbiome of a biological sample. In an aspect, a disclosed microbiome can comprise the gut microbiome, the gastrointestinal microbiome, or both. In an aspect, a disclosed method of reducing the risk of developing a E. coli infection can further comprise obtaining a biological sample from the subject. In an aspect, a disclosed biological sample can comprise a stool sample, a fecal swab, or any combination thereof.

In an aspect of a disclosed method of reducing the risk of developing a E. coli infection, characterizing the microbiome can comprise (i) collecting a biological sample from the subject; (ii) extracting nucleic acid from the subject's biological sample; and (iii) sequencing the extracted nucleic acid. In an aspect, sequencing the extracted nucleic acid can generate sequence data. In an aspect, characterizing the microbiome can further comprise analyzing the sequence data using taxonomic classification. In an aspect, using taxonomic classification can comprise PCR amplification. In an aspect, PCR amplification can comprise using primers targeting the 16S rRNA gene. In an aspect, PCR amplification can comprise primers targeting the V4 variable region of the 16S rRNA gene. In an aspect, disclosed primers can comprise a pair of primers.

In an aspect, a disclosed method of reducing the risk of developing a E. coli infection can further comprise identifying the one or more pathogenic bacteria. In an aspect, a disclosed method of reducing the risk of developing a E. coli infection can further comprise diagnosing the subject with an infection.

In an aspect of a disclosed method of reducing the risk of developing a E. coli infection, a disclosed therapeutically effective dose can comprise a total of at least 100 bacterial CFUs of the one or more probiotics, a total of at least 200 to at least 300 bacterial CFUs of the one or more probiotics, a total of at least 300 to at least 400 bacterial CFUs of the one or more probiotics, a total of at least 400 to at least 500 bacterial CFUs of the one or more probiotics, a total of at least 500 to at least 600 bacterial CFUs of the one or more probiotics, a total of at least 600 to at least 700 bacterial CFUs of the one or more probiotics, a total of at least 700 to at least 800 bacterial CFUs of the one or more probiotics, a total of at least 800 to at least 900 bacterial CFUs of the one or more probiotics, a total of at least 900 to at least 1000 bacterial CFUs of the one or more probiotics, or a total of at least 1000 bacterial CFUs of one or more disclosed probiotics (such as, for example, at least one strain from the bacterial genus Alistipes and/or at least one strain from the bacterial genus Akkermansia).

In an aspect of a disclosed pharmaceutical formulation, a disclosed therapeutically effective dose can comprise a total of at least 1000 to at least 2000 bacterial CFUs of the one or more probiotics, a total of at least 2000 to at least 3000 bacterial CFUs of the one or more probiotics, a total of at least 3000 to at least 4000 bacterial CFUs of the one or more probiotics, a total of at least 4000 to at least 5000 bacterial CFUs of the one or more probiotics, a total of at least 5000 to at least 6000 bacterial CFUs of the one or more probiotics, a total of at least 6000 to at least 7000 bacterial CFUs of the one or more probiotics, a total of at least 8000 to at least 9000 bacterial CFUs of the one or more probiotics, or a total of at least 10,000 bacterial CFUs of one or more disclosed probiotics (such as, for example, at least one strain from the bacterial genus Alistipes and/or at least one strain from the bacterial genus Akkermansia).

In an aspect of a disclosed method of reducing the risk of developing a E. coli infection, a disclosed therapeutically effective dose can comprise a total of at least 100 bacterial CFUs of at least one strain from the bacterial genus Alistipes and/or at least one strain from the bacterial genus Akkermansia, a total of at least 200 to at least 300 bacterial CFUs of at least one strain from the bacterial genus Alistipes and/or at least one strain from the bacterial genus Akkermansia, a total of at least 300 to at least 400 bacterial CFUs of at least one strain from the bacterial genus Alistipes and/or at least one strain from the bacterial genus Akkermansia, a total of at least 400 to at least 500 bacterial CFUs of at least one strain from the bacterial genus Alistipes and/or at least one strain from the bacterial genus Akkermansia, a total of at least 500 to at least 600 bacterial CFUs of at least one strain from the bacterial genus Alistipes and/or at least one strain from the bacterial genus Akkermansia, a total of at least 600 to at least 700 bacterial CFUs of at least one strain from the bacterial genus Alistipes and/or at least one strain from the bacterial genus Akkermansia, a total of at least 700 to at least 800 bacterial CFUs of at least one strain from the bacterial genus Alistipes and/or at least one strain from the bacterial genus Akkermansia, a total of at least 800 to at least 900 bacterial CFUs of at least one strain from the bacterial genus Alistipes and/or at least one strain from the bacterial genus Akkermansia, a total of at least 900 to at least 1000 bacterial CFUs of at least one strain from the bacterial genus Alistipes and/or at least one strain from the bacterial genus Akkermansia, or a total of at least 1000 bacterial CFUs of at least one strain from the bacterial genus Alistipes and/or at least one strain from the bacterial genus Akkermansia.

In an aspect of a disclosed method of reducing the risk of developing a E. coli infection, a disclosed therapeutically effective dose can comprise a total of at least 1000 to at least 2000 bacterial CFUs of at least one strain from the bacterial genus Alistipes and/or at least one strain from the bacterial genus Akkermansia, a total of at least 2000 to at least 3000 bacterial CFUs of at least one strain from the bacterial genus Alistipes and/or at least one strain from the bacterial genus Akkermansia, a total of at least 3000 to at least 4000 bacterial CFUs of at least one strain from the bacterial genus Alistipes and/or at least one strain from the bacterial genus Akkermansia, a total of at least 4000 to at least 5000 bacterial CFUs of at least one strain from the bacterial genus Alistipes and/or at least one strain from the bacterial genus Akkermansia, a total of at least 5000 to at least 6000 bacterial CFUs of at least one strain from the bacterial genus Alistipes and/or at least one strain from the bacterial genus Akkermansia, a total of at least 6000 to at least 7000 bacterial CFUs of at least one strain from the bacterial genus Alistipes and/or at least one strain from the bacterial genus Akkermansia, a total of at least 8000 to at least 9000 bacterial CFUs of at least one strain from the bacterial genus Alistipes and/or at least one strain from the bacterial genus Akkermansia, or a total of at least 10,000 bacterial CFUs of at least one strain from the bacterial genus Alistipes and/or at least one strain from the bacterial genus Akkermansia.

In an aspect of a disclosed method of reducing the risk of developing a E. coli infection, a disclosed therapeutically effective dose can comprise a total of at least 10⁴ to at least 10⁵ bacterial CFUs of the one or more probiotics, a total of at least 10⁵ to at least 10⁶ bacterial CFUs of the one or more probiotics, a total of at least 10⁶ to at least 10⁷ bacterial CFUs of the one or more probiotics, a total of at least 10⁷ to at least 10⁸ bacterial CFUs of the one or more probiotics, a total of at least 10⁸ to at least 10⁹ bacterial CFUs of the one or more probiotics, a total of at least 10⁹ to at least 10¹⁰ bacterial CFUs of the one or more probiotics, a total of at least 10¹⁰ to at least 10¹¹ bacterial CFUs of the one or more probiotics, a total of at least 10¹¹ to at least 10¹² bacterial CFUs of the one or more probiotics, a total of at least 10¹² bacterial CFUs of the one or more probiotics, or more than 10¹² bacterial CFUs of one or more disclosed probiotics (such as, for example, at least one strain from the bacterial genus Alistipes and/or at least one strain from the bacterial genus Akkermansia).

In an aspect of a disclosed method of reducing the risk of developing a E. coli infection, a disclosed therapeutically effective dose can comprise at least 10³, 10⁴, 10⁵, 10⁶, 10⁷, 10⁸, 10⁹, 10¹⁰, 10¹¹, or 10¹² CFUs of a disclosed probiotic or a consortium of disclosed probiotics (such as, for example, at least one strain from the bacterial genus Alistipes and/or at least one strain from the bacterial genus Akkermansia), preferably at least 1.2×10³, 1.4×10³, 2×10³, or 3×10³ CFUs of a disclosed probiotic or a consortium of disclosed probiotics, at least 5×10³, 5.02×10³, 5.04×10³, 5.2×10³, or 5.4×10³ CFUs of a disclosed probiotic or a consortium of disclosed probiotics, at least 1.02×10⁴, 1.04×10⁴, 1.2×10⁴, 1.4×10⁴, 1.5×10⁴, 2×10⁴, or 3×10⁴ CFUs of a disclosed probiotic or a consortium of disclosed probiotics, at least 5×10⁴, 5.02×10⁴, 5.04×10⁴, 5.2×10⁴, or 5.4×10⁴ CFUs of a disclosed probiotic or a consortium of disclosed probiotics, at least 1.02×10⁵, 1.04×10⁵, 1.2×10⁵, 1.4×10⁵, 1.5×10⁵, 2×10⁵, or 3×10⁵ CFUs of a disclosed probiotic or a consortium of disclosed probiotics, at least 1.02×10⁶, 1.04×10⁶, 1.2×10⁶, 1.4×10⁶, 2×10⁶, or 3×10⁶ CFUs of a disclosed probiotic or a consortium of disclosed probiotics, at least 1.02×10⁷, 1.04×10⁷, 1.2×10⁷, 1.4×10⁷, 2×10⁷, or 3×10⁷ CFUs of a disclosed probiotic or a consortium of disclosed probiotics, at least 1.02×10⁸, 1.04×10⁸, 1.2×10⁸, 1.4×10⁸, 2×10⁸, or 3×10⁸ CFUs of a disclosed probiotic or a consortium of disclosed probiotics, at least 1.02×10⁹, 1.04×10⁹, 1.2×10⁹, 1.4×10⁹, 2×10⁹, or 3×10⁹ CFUs of a disclosed probiotic or a consortium of disclosed probiotics, at least 10¹⁰ CFUs of a disclosed probiotic or a consortium of disclosed probiotics, at least 1.02×10¹⁰, 1.04×10¹⁰, 1.2×10¹⁰, 1.4×10¹⁰, 2×10¹⁰, or 3×10¹⁰ CFUs of a disclosed probiotic or a consortium of disclosed probiotics, at least 1.02×10¹¹, 1.04×10¹¹, 1.2×10¹¹, 1.4×10¹¹, 2×10¹¹, or 3×10¹¹ CFUs of a disclosed probiotic or a consortium of disclosed probiotics, or at least 1.02×10¹², 1.04×10¹² 1.2×10¹², 1.4×10¹², 2×10¹², or 3×10¹² CFUs of a disclosed probiotic or a consortium of disclosed probiotics per dose.

In an aspect, a disclosed therapeutically effective dose can comprise any amount or an unlimited amount of bacterial CFUs of the one or more probiotics.

In an aspect, a disclosed method of reducing the risk of developing a pathogenic bacterial and/or ST131 E. coli (i.e., H30R1 and/or H30Rx) infection can further comprise treating the subject. In an aspect, treating the subject can comprise treating the subject's respiratory infection. In an aspect, treating the subject can comprise treating the subject's non-respiratory infection.

In an aspect, a disclosed method of reducing the risk of developing a E. coli infection can comprise further comprising administering to the subject a therapeutically effective amount of one or more anti-bacterial agents. Anti-bacterial agents and combinations of anti-bacterial agents are known to the art and discussed supr.

In an aspect, a disclosed method of reducing the risk of developing a E. coli infection can comprise further comprising administering to the subject a therapeutically effective amount of (i) one or more active agents, (ii) one or more biologically active agents, (iii) one or more pharmaceutically active agents, (iv) one or more immune-based therapeutic agents, (v) one or more clinically approved agents, or (vi) any combination thereof. In an aspect, a therapeutically effective amount of an active agent, a biologically active agent, a pharmaceutically active agent, an immune-based therapeutic active agent, a clinically approved agent, or an anti-bacterial agent can be about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, a disclosed method of reducing the risk of developing a E. coli infection can further comprise administering to the subject one or more therapeutic agents. In an aspect, a disclosed therapeutic agent can comprise a biologically active agent, a pharmaceutically active agent, an anti-bacterial agent, an anti-fungal agent, an anti-viral agent, a corticosteroid, an analgesic, an immunostimulant, an immune-based product, or any combination thereof. In an aspect of a disclosed method of promoting respiratory health, administering can comprise intranasal administration, oral administration, sublingual administration, or any combination thereof.

In an aspect of a disclosed method of reducing the risk of developing a E. coli infection, a subject can be a healthy subject, for example, a subject that does not have an infection and/or is not suspected of having an infection. In an aspect, a subject can be an adult, a child, or an infant. In an aspect, a subject can be a neonate. In an aspect, a subject can be a premature infant. In an aspect, a subject can be immune-compromised. In an aspect, a subject can have diabetes or a chronic disease (e.g., heart disease, kidney disease, or liver disease). In an aspect, a subject can have HIV. In an aspect, a subject can have cancer or has had cancer. In an aspect, a subject can be the recipient of one or more solid organ transplants. In an aspect, a female subject can be pregnant. In an aspect, a subject can have or can be suspected of having a urinary tract infection (UTI). In an aspect, a subject can be in a high-risk environment (e.g., an acute care facility, a hospital, a longer-term acute care hospital, an in-patient facility, an in-patient rehabilitation facility, or a battle-zone or active war zone). In an aspect, a subject can be in the midst of a humanitarian crisis.

In an aspect, a subject can be diagnosed with an infection of one or more parts of the respiratory system. In an aspect, a disclosed therapeutically effective amount of a biotherapeutic can comprise an amount sufficient to inhibit and/or prevent the growth of one or more pathogenic bacteria in one or more parts of the subject's gastrointestinal system.

In an aspect, a disclosed method of reducing the risk of developing a pathogenic E. coli infection can comprise a 10%, 20%, 30%, 40%, 50%, 60%, 70%, 80%, 90%, 100%, or any amount of inhibiting and/or preventing the growth and/or spread of one or more pathogenic bacteria (e.g., ST131 (H30R1 and/or H30Rx)) when compared to a control subject (such as, for example, a subject that has not received a disclosed biotherapeutic). In an aspect, a disclosed method of reducing the risk of developing a pathogenic E. coli infection can comprise a 10-20%, 20-30%, 30-40%, 40-50%, 50-60%, 60-70%, 70-80%, 80-90%, or 90-100% or any amount of inhibiting and/or preventing the growth and/or spread of one or more pathogenic bacteria (e.g., ST131 (H30R1 and/or H30Rx)) when compared to a control subject (such as, for example, a subject that has not received a disclosed biotherapeutic).

In an aspect, a disclosed method of reducing the risk of developing a pathogenic E. coli infection can comprise a 10%, 20%, 30%, 40%, 50%, 60%, 70%, 80%, 90%, 100%, or any amount of inhibiting and/or preventing the adherence and/or colonization of one or more pathogenic bacteria (e.g., ST131 (H30R1 and/or H30Rx)) when compared to a control subject (such as, for example, a subject that has not received a disclosed biotherapeutic). In an aspect, a disclosed method of reducing the risk of developing a pathogenic E. coli infection can comprise a 10-20%, 20-30%, 30-40%, 40-50%, 50-60%, 60-70%, 70-80%, 80-90%, or 90-100% or any amount of inhibiting and/or preventing the adherence and/or colonization of one or more pathogenic bacteria (e.g., ST131 (H30R1 and/or H30Rx)) when compared to a control subject (such as, for example, a subject that has not received a disclosed biotherapeutic).

In an aspect, a disclosed method of reducing the risk of developing a pathogenic E. coli infection can comprise a 10%, 20%, 30%, 40%, 50%, 60%, 70%, 80%, 90%, 100%, or any amount of modulating the endocytosis or paracellular migration of one or more pathogenic bacteria (e.g., ST131 E. coli (i.e., H30R1 and/or H30Rx)) when compared to a control subject (such as, for example, a subject that has not received a disclosed biotherapeutic). In an aspect, a disclosed method of reducing the risk of developing a pathogenic E. coli infection can comprise a 10-20%, 20-30%, 30-40%, 40-50%, 50-60%, 60-70%, 70-80%, 80-90%, or 90-100% or any amount of modulating the endocytosis or paracellular migration of one or more pathogenic bacteria (e.g., ST131 E. coli (i.e., H30R1 and/or H30Rx)) when compared to a control subject (such as, for example, a subject that has not received a disclosed biotherapeutic).

In an aspect, a disclosed method of reducing the risk of developing a pathogenic E. coli infection can comprise a 10%, 20%, 30%, 40%, 50%, 60%, 70%, 80%, 90%, 100%, or any amount of inhibiting and/or preventing the spread of one or more pathogenic bacteria (e.g., ST131 (H30R1 and/or H30Rx) by competitive adherence and/or niche occupancy when compared to a control subject (such as, for example, a subject that has not received a disclosed biotherapeutic). In an aspect, a disclosed method of reducing the risk of developing a pathogenic E. coli infection can comprise a 10-20%, 20-30%, 30-40%, 40-50%, 50-60%, 60-70%, 70-80%, 80-90%, or 90-100% or any amount of inhibiting and/or preventing the spread of one or more pathogenic bacteria (e.g., ST131 (H30R1 and/or H30Rx)) by competitive adherence and/or niche occupancy when compared to a control subject (such as, for example, a subject that has not received a disclosed biotherapeutic).

In an aspect, a disclosed method of reducing the risk of developing a pathogenic E. coli infection can comprise repeating the administering of one or more disclosed biotherapeutics, one or more disclosed secreted factors, a disclosed consortium of probiotics, one or more disclosed probiotics, or any combination thereof. For example, in an aspect, repeating the administering can comprise administering one or more times daily (e.g., 1, 2, 3, or 4 times). In an aspect, repeating the administering can comprise administering on several consecutive days (e.g., for 1, 2, 3, 4, 5, 6, 7, 8, 9, or 10 days, or more than 10 days), weeks (e.g., for 1, 2, 3, 4, 5, 6, 7, 8, 9, or 10 weeks, or more than 10 weeks), or months (e.g., for 1, 2, 3, 4, 5, or 6 months, or more than 6 months). In an aspect, a disclosed biotherapeutic, a disclosed secreted factor, a disclosed consortium of probiotics, a disclosed probiotic, or any combination thereof can be administered 1-3 times per day for about 7 to 21 days.

In an aspect, a disclosed method of reducing the risk of developing a pathogenic E. coli infection can comprise monitoring the subject. In an aspect, monitoring the subject can comprise monitoring the subject for the development of adverse effects. In an aspect, in the absence of adverse effects, a disclosed method of reducing the risk of developing a pathogenic E. coli infection can comprise continuing to treat the subject. In an aspect, continuing to treat the subject can comprise continuing to administer to the subject one or more disclosed biotherapeutics, one or more disclosed secreted factors, a disclosed consortium of probiotics, one or more disclosed probiotics, or any combination thereof. In an aspect, in the presence of adverse effects, a disclosed method of reducing the risk of developing a pathogenic E. coli infection can comprise modifying one or more steps of the method. In an aspect, modifying one or more steps of a disclosed method can comprise modifying the administering step. In an aspect, modifying the administering step can comprise changing the amount of one or more disclosed biotherapeutics, one or more disclosed secreted factors, a disclosed consortium of probiotics, one or more disclosed probiotics, or any combination thereof administered to the subject, changing the frequency of administration of one or more disclosed biotherapeutics, one or more disclosed secreted factors, a disclosed consortium of probiotics, one or more disclosed probiotics, or any combination thereof, changing the duration of administration of one or more disclosed biotherapeutics, one or more disclosed secreted factors, a disclosed consortium of probiotics, one or more disclosed probiotics, or any combination thereof, changing the route of administration of one or more disclosed biotherapeutics, one or more disclosed secreted factors, a disclosed consortium of probiotics, one or more disclosed probiotics, or any combination thereof, or any combination of disclosed changes.

In an aspect, a disclosed method of reducing the risk of developing a pathogenic E. coli infection can comprise manipulating the microbiome to inhibit and/or prevent infection caused by one or more pathogenic bacteria, such as an infection caused by ST131 E. coli (i.e., H30R1 and/or H30Rx). In an aspect, a disclosed method of reducing the risk of developing a pathogenic E. coli infection can comprise manipulating the microbiome to decrease and/or minimize the risk of infection caused by one or more pathogenic bacteria, such as an infection caused by ST131 E. coli (i.e., H30R1 and/or H30Rx).

In an aspect, a disclosed method of reducing the risk of developing a pathogenic E. coli infection can comprise subjecting the subject to one or more invasive or non-invasive diagnostic assessments. Diagnostic assessments are known to the art. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination thereof. In an aspect, a disclosed invasive diagnostic assessment can comprise a tissue biopsy or exploratory surgery.

In an aspect of a disclosed method of reducing the risk of developing a pathogenic E. coli infection, techniques to monitor, measure, and/or assess the restoring one or more aspects of respiratory health can comprise qualitative (or subjective) means as well as quantitative (or objective) means. These means are known to the skilled person. For example, representative regulated variables and sensors relating to systemic homeostasis are discussed supra.

In an aspect of a disclosed method of reducing the risk of developing a pathogenic E. coli infection, a disclosed composition, a disclosed pharmaceutical formulation, a disclosed biotherapeutic, a disclosed bacteria belonging to Alistipes genus and/or a disclosed bacteria belonging to Akkermansia, a disclosed secreted factor, a disclosed consortium of probiotics, a disclosed probiotic (e.g., Alistipes and/or Akkermansia), or any combination thereof can be administered in the absence of identifying the one or more pathogenic bacteria and/or in the absence of characterizing the microbiome. In an aspect of a disclosed method, a disclosed composition, a disclosed pharmaceutical formulation, a disclosed biotherapeutic, a disclosed bacteria belonging to Alistipes genus and/or a disclosed bacteria belonging to Akkermansia, a disclosed secreted factor, a disclosed consortium of probiotics, a disclosed probiotic (e.g., Alistipes and/or Akkermansia), or any combination thereof can be administered on demand.

In an aspect of a disclosed method of reducing the risk of developing a pathogenic E. coli infection, a disclosed biotherapeutic can be formulated for administration as a pharmaceutical formulation. In an aspect, a disclosed pharmaceutical formulation comprising a probiotic, a consortium of probiotics, factors secreted from a probiotic, factors secreted from a consortium of probiotics, or any combination thereof, and a pharmaceutically acceptable carrier and/or excipient can be used in any disclosed method of reducing the risk of developing a pathogenic E. coli infection. Any pharmaceutical formulation disclosed herein can be used in a disclosed method of reducing the risk of developing a pathogenic ST131 E. coli infection (i.e., H30R1 and/or H30Rx).

In an aspect, a disclosed method can further (i) reduce the risk of developing a colonization of drug-resistant E. coli, (ii) reduce the degree and/or severity of a drug-resistant E. coli infection, (iii) reduce the degree and/or severity of a drug-resistant E. coli colonization, (iv) reduce the degree and/or severity of a drug-resistant E. coli colonization, or (v) any combination thereof.

In an aspect, a disclosed method can further comprise administering to the subject one or more broad-spectrum antibiotic or antibiotic agents.

D. Methods of Reducing the Degree and/or Severity of a Drug-Resistant E. coli Infection

Disclosed herein is a method of reducing the degree and/or severity of a drug-resistant E. coli infection in a subject, the method comprising administering to a subject in need thereof a therapeutically effective amount of a composition comprising one or more bacteria belonging to the Alistipes genus and/or Akkermansia genus, wherein following the administering step the composition reduces the intestinal carriage and/or persistence of one or more E. coli bacteria, thereby reducing the degree and/or severity of an infection of a drug-resistant E. coli.

Disclosed herein is a method of reducing the degree and/or severity of a drug-resistant E. coli colonization in a subject, the method comprising administering to a subject in need thereof a therapeutically effective amount of a composition comprising one or more bacteria belonging to the Alistipes genus and/or Akkermansia genus, wherein following the administering step the composition reduces the intestinal carriage and/or persistence of one or more E. coli bacteria, thereby reducing the degree and/or severity of colonization by a drug-resistance E. coli.

Disclosed herein is a method of reducing the degree and/or severity of a drug-resistant E. coli colonization in a subject, the method comprising administering to a subject in need thereof a therapeutically effective amount of a composition that modulates the intestinal carriage associated with the colonization of drug-resistant E. coli bacteria, wherein, following the administering step, the composition reduces the proportional abundance of one or more bacteria associated with the carriage and/or persistence of drug-resistant E. coli bacteria, thereby reducing the degree and/or severity of colonization by a drug-resistance E. coli.

In an aspect, one or more disclosed E. coli bacteria can comprise sequence type 131 (ST131). In an aspect, one or more disclosed ST131 E. coli bacteria can comprise a H30R subclone. In an aspect, a disclosed H30R subclone can comprise H30R1 or H30Rx. In an aspect, one or more disclosed E. coli bacteria can be multi-drug resistant. In an aspect, one or more disclosed E. coli bacteria are fluoroquinolone resistant. In an aspect, one or more disclosed E. coli bacteria can comprise sequence type 131 (ST131) and can be multi-drug resistant. In an aspect, one or more disclosed ST131 E. coli bacteria can comprise a H30R subclone and can be multi-drug resistant. In an aspect, one or more disclosed H30R subclones can comprise H30R1 or H30Rx and can be multi-drug resistant. In an aspect, one or more disclosed E. coli bacteria can comprise sequence type 131 (ST131) E. coli bacteria and can be fluoroquinolone resistant. In an aspect, one or more disclosed ST131 E. coli bacteria can comprise a H30R subclone and can be fluoroquinolone resistant. In an aspect, one or more disclosed H30R subclones can comprise H30R1 or H30Rx and can be fluoroquinolone resistant.

In an aspect, a disclosed composition can comprise both one or more bacteria belonging to the Alistipes genus and one or more bacteria belonging to the Akkermansia genus. In an aspect, a disclosed composition can comprise one or more bacteria belonging to the Alistipes genus. In an aspect, a disclosed composition can comprise one or more bacteria belonging to an addition genus. In an aspect, a disclosed composition can comprise one or more bacterial belonging to the Alistipes genus and one or more bacteria belonging to the Akkermansia genus. In an aspect, a disclosed composition can comprise one or more bacteria belonging to the Alistipes genus. In an aspect, the Alistipes genus can comprise Alistipes finegoldii, Alistipes putredinis, Alistipes onderdonkii, Alistipes shahii, Alistipes indistinctus, Alistipes senegalensis, Alistipes timonensis, Alistipes obesi, Alistipes ihumii, Alistipes inops, Alistipes megaguti, Alistipes provencensis, Alistipes massiliensis, any combination thereof. In an aspect, a disclosed composition can comprise one or more bacterial species belonging to the Verrucomicrobia phylum. In an aspect, a disclosed composition can comprise one or more bacterial belonging to the Alistipes genus and one or more bacteria belonging to the Akkermansia genus.

In an aspect, a disclosed method can further comprise administering to the subject one or more bacteriophages capable of killing E. coli. In an aspect, a disclosed E. coli-killing bacteriophage can comprise BP539, BP700, BP753, BP814, BP953, BP954, BP970, BP1002, BP1151, BP1155, BP1168, BP1176, BP1197, BP1226, BP1229, or any combination thereof. In an aspect, a disclosed E. coli-killing bacteriophage can comprise MV36.2, JJ2050.2, C3, C19T, JJ6.1, or any combination thereof.

In an aspect, a disclosed composition can comprise one or more agents that can reduce the intestinal carriage associated with the colonization of drug-resistant E. coli bacteria, thereby reducing the risk of an infection. In an aspect, a disclosed composition can comprise one or more agents that can reduce the proportional abundance of one or more bacteria associated with the carriage and/or persistence of drug-resistant E. coli bacteria, thereby reducing the degree and/or severity of colonization by a drug-resistance E. coli. In an aspect, a disclosed composition can comprise one or more agents that can inhibit proliferation of Asaccharobacter, Bifidobacterium, Collinsella, Eggerthella, Gardnerella, Gordonibacter, or any combination thereof in the gut microbiome of the subject. In an aspect, a disclosed composition can comprise one or more agents that can increase proliferation of Alistipes and/or Akkermansia.

In an aspect, a disclosed composition can comprise (i) one or more agents that can reduce the intestinal carriage associated with the colonization of drug-resistant E. coli bacteria, (ii) one or more agents that can reduce the proportional abundance of one or more bacteria associated with the carriage and/or persistence of drug-resistant E. coli bacteria, (iii) one or more agents that can inhibit proliferation of Asaccharobacter, Bifidobacterium, Collinsella, Eggerthella, Gardnerella, Gordonibacter, or any combination thereof in the gut microbiome of the subject, (iv) one or more agents that can increase proliferation of Alistipes and/or Akkermansia, or (v) any combination thereof.

In an aspect, relative species abundance is a component of biodiversity. In an aspect, relative species abundance is a measure of how common or rare a species is relative to other species in a defined location or community. In an aspect, relative species abundance is a measure of how common or rare a species is relative to other species in a subject's gut microbiome. In an aspect, relative species abundance is a measure of how common or rare a species is relative to other species in a subject's gut microbiome prior to a disclosed administering step. In an aspect, relative species abundance is a measure of how common or rare a species is relative to other species in a subject's gut microbiome following a disclosed administering step. In an aspect, relative abundance is the percent composition of an organism of a particular kind relative to the total number of organisms in the area. In an aspect, relative abundance is the percent composition of an organism of a particular kind relative to the total number of organisms in a subject's gut microbiome. In an aspect, relative abundance is the percent composition of an organism of a particular kind relative to the total number of organisms in a subject's gut microbiome prior to a disclosed administering step. In an aspect, relative abundance is the percent composition of an organism of a particular kind relative to the total number of organisms in a subject's gut microbiome following a disclosed administering step.

In an aspect, abundance can be the number of individuals of a given species in a region. In an aspect, incidence can be the number of samples containing at least one of a given species in a census. In an aspect, sampling scope can be the area, interval of time, and taxonomic grouping over which sampling takes place. In an aspect, and in the context of community sampling, spatial heterogeneity can be variation in species capture probabilities across space. In an aspect, spatial heterogeneity can be due to habitat variation, intraspecific clumping, interspecific association, other factors, or any combination thereof. In an aspect, species abundance distribution can be the number of species found in each interval of abundance in a community.

In an aspect, species accumulation curve can be a plot of the total number of species observed in a census against some measure of cumulative sampling effort. In an aspect, species accumulation can be any of a number of measures concerning the number of species in an area and/or the distribution of their abundances. In an aspect, species richness can be the number of species present in a region.

In an aspect, and in the context of community sampling, temporal heterogeneity can be variation in species capture probabilities over time. In an aspect, temporal heterogeneity can arise from temporal environmental variation, migration, speciation or other factors. In an aspect, bacterial diversity can comprise two components: (i) species richness (i.e., the number of species in the community), and (ii) evenness (i.e., the fact that some species in the community are common and others are rare). In an aspect, an important component of most diversity indices is a value expressing the relative proportion of the species in the community (pi). In an aspect, this value can be calculated as the absolute (raw) abundance or dominance of the species (i) in the community, divided by the sum of all species abundances/dominances in that community. In an aspect, the sum of relative abundances for all species in the community equals to unity.

In an aspect of a disclosed method of reducing the degree and/or severity of a drug-resistant E. coli colonization in a subject, prior to the administering step the gut microbiome of the subject can comprise a higher proportional abundance of one or more bacteria belonging to the Actinobacteria phylum than that of a control subject. In an aspect, Actinobacteria phylum can comprise Asaccharobacter, Bifidobacterium, Collinsella, Eggerthella, Gardnerella, Gordonibacter, or any combination thereof. In an aspect of a disclosed method of reducing the degree and/or severity of a drug-resistant E. coli colonization in a subject, prior to the administering step the gut microbiome of the subject can comprise a higher proportional abundance of Collinsella than that of a control subject. In an aspect of a disclosed method of reducing the degree and/or severity of a drug-resistant E. coli colonization in a subject, prior to the administering step the gut microbiome of the subject can comprise a higher proportional abundance of Gardnerella and/or Gordonibacter than that of a control subject. In an aspect of a disclosed method of reducing the degree and/or severity of a drug-resistant E. coli colonization in a subject, prior to the administering step the gut microbiome of the subject can comprise a higher proportional abundance of one or more bacteria belonging to the Firmicutes phylum than that of a control subject. In an aspect, Firmicutes phylum can comprise Enterococcus, Lactobacillus, Streptococcus, Weissella, or any combination thereof. In an aspect of a disclosed method of reducing the degree and/or severity of a drug-resistant E. coli colonization in a subject, prior to the administering step the gut microbiome of the subject can comprise a higher proportional abundance of Lactobacillus and/or Streptococcus than that of a control subject.

In an aspect of a disclosed method of reducing the degree and/or severity of a drug-resistant E. coli colonization in a subject, prior to the administering step the gut microbiome of the subject can comprise a lower proportional abundance of one or more bacteria belonging to the Bacteroidetes phylum than that of a control subject. In an aspect, Bacteroidetes phylum can comprise Alistipes and/or Bacteroides. In an aspect, Bacteroides can comprise B. dorei, B. uniformis, B. eggerthii, B. stercoris, B. fragilis, B. caccae, B. intestinalis, B. ovatus, B. xylanisolvens, B. splanchnicus, or any combination thereof. In an aspect, Alistipes can comprise Alistipes finegoldii, Alistipes putredinis, Alistipes onderdonkii, Alistipes shahii, Alistipes indistinctus, Alistipes senegalensis, Alistipes timonensis, Alistipes obesi, Alistipes ihumii, Alistipes inops, Alistipes megaguti, Alistipes provencensis, Alistipes massiliensis, any combination thereof.

In an aspect, measuring the prevalence and/or proportional abundance of one or more bacteria in the subject's gut microbiome can inform a clinician's treatment decisions.

In an aspect, prior to the administering step the gut microbiome of the subject can comprise a lower proportional abundance of Bacteroides than that of a control subject. In an aspect, prior to the administering step the gut microbiome of the subject can comprise a lower proportional abundance of Alistipes than that of a control subject. In an aspect, prior to the administering step the gut microbiome of the subject can comprise a lower proportional abundance of one or more bacteria belonging to Verrucomicrobia phylum than that of a control subject. In an aspect, Verrucomichrobia phylum can comprise family Akkermansiaceae and family Verrucomicrobiaceae. In an aspect, Akkermansiaceae can comprise Akkermansia. In an aspect of a disclosed method of reducing the degree and/or severity of a drug-resistant E. coli colonization in a subject, wherein prior to the administering step the gut microbiome of the subject can comprise a lower proportional abundance of Akkermansia than that of a control subject.

In an aspect of a disclosed method of reducing the degree and/or severity of a drug-resistant E. coli colonization in a subject, wherein following the administering step, the proportional abundance of one or more E. coli sequence type 131 (ST131) bacteria in the gut microbiome of the subject is decreased. In an aspect of a disclosed method of reducing the degree and/or severity of a drug-resistant E. coli colonization in a subject, wherein, following the administering step, the proportional abundance of ST131-H30R1 in the gut microbiome of the subject is decreased. In an aspect of a disclosed method of reducing the degree and/or severity of a drug-resistant E. coli colonization in a subject, wherein, following the administering step, the proportional abundance of ST131-H30Rx in the gut microbiome of the subject is decreased. In an aspect of a disclosed method of reducing the degree and/or severity of a drug-resistant E. coli colonization in a subject, wherein, following the administering step, the proportional abundance of ST131-H30R1 and ST131-H30Rx in the gut microbiome of the subject is decreased. In an aspect of a disclosed method of reducing the degree and/or severity of a drug-resistant E. coli colonization in a subject, wherein, following the administering step, the level of and/or the amount of ST131-H30R1 and/or the level of and/or the amount of ST131-H30Rx in the gut microbiome of the subject is decreased from the level and/or the amount prior to the administering step.

In an aspect of a disclosed method of reducing the degree and/or severity of a drug-resistant E. coli colonization in a subject, wherein, following the administering step, the proportional abundance of one or more bacteria belonging to Actinobacteria phylum in the gut microbiome of the subject is decreased. In an aspect of a disclosed method of reducing the degree and/or severity of a drug-resistant E. coli colonization in a subject, wherein, following the administering step, the proportional abundance of Asaccharobacter, Bifidobacterium, Collinsella, Eggerthella, Gardnerella, Gordonibacter, or any combination thereof in the gut microbiome of the subject is decreased. In an aspect of a disclosed method of reducing the degree and/or severity of a drug-resistant E. coli colonization in a subject, wherein, following the administering step, the proportional abundance of Collinsella in the gut microbiome of the subject is decreased. In an aspect of a disclosed method of reducing the degree and/or severity of a drug-resistant E. coli colonization in a subject, following the administering step, the proportional abundance of Gardnerella and/or Gordonibacter in the gut microbiome of the subject is decreased. In an aspect of a disclosed method of reducing the degree and/or severity of a drug-resistant E. coli colonization in a subject, wherein, following the administering step, the proportional abundance of one or more bacteria belonging to Firmicutes phylum in the gut microbiome of the subject is decreased.

In an aspect of a disclosed method of reducing the degree and/or severity of a drug-resistant E. coli colonization in a subject, wherein, following the administering step, the proportional abundance of Enterococcus, Lactobacillus, Streptococcus, Weissella, or any combination thereof in the gut microbiome of the subject is decreased. In an aspect of a disclosed method of reducing the degree and/or severity of a drug-resistant E. coli colonization in a subject, wherein, following the administering step, the proportional abundance of Lactobacillus and/or Streptococcus in the gut microbiome of the subject is decreased.

In an aspect of a disclosed method of reducing the degree and/or severity of a drug-resistant E. coli colonization in a subject, wherein, following the administering step, the level of and/or the amount of ST131-H30R1 and/or ST131-H30Rx in the gut microbiome of the subject is decreased from the level and/or the amount prior to the administering step. In an aspect of a disclosed method of reducing the degree and/or severity of a drug-resistant E. coli colonization in a subject, wherein, following the administering step, the level of and/or the amount of one or more bacteria belonging to the Actinobacteria phylum in the gut microbiome of the subject is decreased from the level and/or the amount prior to the administering step.

In an aspect of a disclosed method of reducing the degree and/or severity of a drug-resistant E. coli colonization in a subject, wherein, following the administering step, the level of and/or the amount of one or more bacteria belonging to the Actinobacteria phylum in the gut microbiome of the subject is decreased from the level and/or the amount prior to the administering step. In an aspect of a disclosed method of reducing the degree and/or severity of a drug-resistant E. coli colonization in a subject, wherein, following the administering step, the level of and/or the amount of Asaccharobacter, Bifidobacterium, Collinsella, Eggerthella, Gardnerella, Gordonibacter, or any combination thereof in the gut microbiome of the subject is decreased from the level and/or the amount prior to the administering step. In an aspect of a disclosed method of reducing the degree and/or severity of a drug-resistant E. coli colonization in a subject, wherein, following the administering step, the level of and/or the amount Collinsella in the gut microbiome of the subject is decreased from the level and/or the amount prior to the administering step.

In an aspect of a disclosed method of reducing the degree and/or severity of a drug-resistant E. coli colonization in a subject, wherein, following the administering step, the level of and/or the amount of Gardnerella and/or Gordonibacter in the gut microbiome of the subject is decreased from the level and/or the amount prior to the administering step.

In an aspect of a disclosed method of reducing the degree and/or severity of a drug-resistant E. coli colonization in a subject, wherein, following the administering step, the level of and/or the amount of one or more bacteria belonging to the Firmicutes phylum in the gut microbiome of the subject is decreased from the level and/or the amount prior to the administering step. In an aspect of a disclosed method of reducing the degree and/or severity of a drug-resistant E. coli colonization in a subject, wherein, following the administering step, the level of and/or the amount of Enterococcus, Lactobacillus, Streptococcus, Weissella, or any combination thereof in the gut microbiome of the subject is decreased from the level and/or the amount prior to the administering step. In an aspect of a disclosed method of reducing the degree and/or severity of a drug-resistant E. coli colonization in a subject, wherein, following the administering step, the level of and/or the amount of Lactobacillus and/or Streptococcus in the gut microbiome of the subject is decreased from the level and/or the amount prior to the administering step.

In an aspect, gastrointestinal administration can comprise oral, nasogastric, or rectal administration. In an aspect, a disclosed composition can comprise a liquid, a suspension, a dried powder, a tablet, a capsule, or a food product.

In an aspect, a disclosed method of reducing the degree and/or severity of a drug-resistant E. coli colonization in a subject can further comprise administering to the subject an antibiotic, an anti-toxin antibody, an herbal remedy, a probiotic bacteria, a probiotic yeast, or any combination thereof. In an aspect, a disclosed method of reducing the degree and/or severity of a drug-resistant E. coli colonization in a subject can further comprise administering to the subject one or more antibiotics, anti-toxin antibodies, herbal remedies, probiotic bacteria, probiotic yeast, or any combination thereof. In an aspect, a disclosed method of reducing the degree and/or severity of a drug-resistant E. coli colonization in a subject can further comprise administering to the subject antibiotic, an anti-toxin antibody, an herbal remedy, a probiotic bacteria, a probiotic yeast, or any combination thereof.

In an aspect of a disclosed method of reducing the degree and/or severity of a drug-resistant E. coli colonization in a subject, the therapeutically effective amount can ameliorate at least one symptom of an E. coli infection. In an aspect, a disclosed symptom of an E. coli infection can comprise abdominal tenderness, abdominal pain, abdominal cramping, sepsis, endocarditis, meningitis, headache, stiff neck, confusion, back pain, pneumonia, fever, chills, diarrhea, urinary tract infection, endocarditis, elevated white blood cell count, and decreased serum albumin.

In an aspect, a disclosed therapeutically effective amount can inhibit proliferation of one or more E. coli bacteria in the gastrointestinal tract of the subject. In an aspect, a disclosed therapeutically effective amount can inhibit proliferation of one or more E. coli bacteria in the gut microbiome of the subject.

In an aspect, a disclosed therapeutically effective amount can inhibit proliferation of E. coli bacteria can comprise sequence type 131 (ST131) in the gastrointestinal tract of the subject. In an aspect, a disclosed therapeutically effective amount can inhibit proliferation of E. coli bacteria can comprise sequence type 131 (ST131) in the gut microbiome of the subject. In an aspect, a disclosed therapeutically effective amount can inhibit proliferation of H30R in the gastrointestinal tract of the subject. In an aspect, a disclosed therapeutically effective amount can inhibit proliferation of H30R in the gut microbiome of the subject.

In an aspect, a disclosed therapeutically effective amount can inhibit proliferation of H30Rx in the gastrointestinal tract of the subject. In an aspect, a disclosed therapeutically effective amount can inhibit proliferation of H30Rx in the gut microbiome of the subject.

In an aspect, a disclosed therapeutically effective amount can inhibit proliferation of one or more bacteria belonging to Actinobacteria phylum in the gastrointestinal tract of the subject. In an aspect, a disclosed therapeutically effective amount can inhibit proliferation of one or more bacteria belonging to Actinobacteria phylum in the gut microbiome of the subject.

In an aspect, a disclosed therapeutically effective amount can inhibit proliferation of Asaccharobacter, Bifidobacterium, Collinsella, Eggerthella, Gardnerella, Gordonibacter, or any combination thereof in the gastrointestinal tract of the subject. In an aspect, a disclosed therapeutically effective amount can inhibit proliferation of Asaccharobacter, Bifidobacterium, Collinsella, Eggerthella, Gardnerella, Gordonibacter, or any combination thereof in the gut microbiome of the subject.

In an aspect, a disclosed therapeutically effective amount can inhibit proliferation of Collinsella in the gastrointestinal tract of the subject. In an aspect, a disclosed therapeutically effective amount can inhibit proliferation of Collinsella in the gut microbiome of the subject.

In an aspect, a disclosed therapeutically effective amount can inhibit proliferation of Gardnerella and/or Gordonibacter in the gastrointestinal tract of the subject. In an aspect, a disclosed therapeutically effective amount can inhibit proliferation of Gardnerella and/or Gordonibacter in the gut microbiome of the subject.

In an aspect, a disclosed therapeutically effective amount can inhibit proliferation of one or more bacteria belonging to Firmicutes phylum in the gastrointestinal tract of the subject. In an aspect, a disclosed therapeutically effective amount can inhibit proliferation of one or more bacteria belonging to Firmicutes phylum in the gut microbiome of the subject.

In an aspect, a disclosed therapeutically effective amount can inhibit proliferation of Enterococcus, Lactobacillus, Streptococcus, Weissella, or any combination thereof in the gastrointestinal tract of the subject. In an aspect, a disclosed therapeutically effective amount can inhibit proliferation of Enterococcus, Lactobacillus, Streptococcus, Weissella, or any combination thereof in the gut microbiome of the subject.

In an aspect, a disclosed therapeutically effective amount can inhibit proliferation of Lactobacillus and/or Streptococcus in the gastrointestinal tract of the subject. In an aspect, a disclosed therapeutically effective amount can inhibit proliferation of Lactobacillus and/or Streptococcus in the gut microbiome of the subject.

In an aspect of a disclosed method of reducing the degree and/or severity of a drug-resistant E. coli colonization in a subject, wherein following the administering step, the subject can be now susceptible and/or more susceptible to fluoroquinolone. In an aspect of a disclosed method of reducing the degree and/or severity of a drug-resistant E. coli colonization in a subject, wherein following the administering step, the subject can be now susceptible and/or more susceptible to antibiotic treatment. In an aspect of a disclosed method of reducing the degree and/or severity of a drug-resistant E. coli colonization in a subject, wherein following the administering step, the subject can be now susceptible and/or more susceptible to a treatment regimen comprising one or more antibiotic agents.

In an aspect, a disclosed method of reducing the degree and/or severity of a drug-resistant E. coli colonization in a subject can further comprise measuring the prevalence and/or proportional abundance of one or more Escherichia coli (E. coli) bacteria. In an aspect, E. coli bacteria can comprise sequence type 131 (ST131). In an aspect, ST131 E. coli bacteria can comprise a H30R subclone. In an aspect, a disclosed H30R subclone can comprise H30R1 or H30Rx. In an aspect, a disclosed E. coli bacteria can comprise sequence type 131 (ST131) and can be multi-drug resistant. In an aspect, a disclosed ST131 E. coli bacteria can comprise a H30R subclone and can be multi-drug resistant. In an aspect, a disclosed H30R subclone can comprise H30R1 or H30Rx and can be multi-drug resistant. In an aspect, a disclosed E. coli bacteria can be multi-drug resistant. In an aspect, a disclosed E. coli bacteria can be fluoroquinolone resistant. In an aspect, a disclosed E. coli bacteria can comprise sequence type 131 (ST131) E. coli bacteria and can be fluoroquinolone resistant. In an aspect, a disclosed ST131 E. coli bacteria can comprise a H30R subclone and can be fluoroquinolone resistant. In an aspect, a disclosed H30R subclone can comprise H30R1 or H30Rx and can be fluoroquinolone resistant.

In an aspect, a disclosed method of reducing the degree and/or severity of a drug-resistant E. coli colonization in a subject can further comprise obtaining one or more biological samples from the subject. In an aspect, a disclosed method of reducing the degree and/or severity of a drug-resistant E. coli colonization in a subject can further comprise repeating the obtaining of one or more biological samples from the subject.

In an aspect, the repeating of the obtaining step can occur at different times. In an aspect, the first obtaining step can be performed prior to administering any treatment to the subject. In an aspect, the second obtaining step can be performed after administering any treatment to the subject. In an aspect, subsequent obtaining steps can be performed after administering any treatment to the subject.

In an aspect, a disclosed method of reducing the degree and/or severity of a drug-resistant E. coli colonization in a subject can further comprise comparing the prevalence and/or proportional abundance of one or more bacteria in the subject's gut microbiome to one or more standard levels. In an aspect, a disclosed method of reducing the degree and/or severity of a drug-resistant E. coli colonization in a subject can further comprise comparing the prevalence and/or proportional abundance of one or more bacteria in the subject's gut microbiome to one or more control levels.

In an aspect, a disclosed method of reducing the degree and/or severity of a drug-resistant E. coli colonization in a subject can further comprise comparing the prevalence and/or proportional abundance of the one or more bacteria in the subject's gut microbiome to one or more standard levels, and if the prevalence and/or proportional abundance of the one or more bacteria in the subject's gut microbiome is similar to that of one or more subjects having demonstrated responsiveness to treatment, then characterizing the subject as likely to be responsive to treatment.

In an aspect, a disclosed method of reducing the degree and/or severity of a drug-resistant E. coli colonization in a subject can further comprise comparing the prevalence and/or proportional abundance of the one or more bacteria in the subject's gut microbiome to one or more standard levels, and if the prevalence and/or proportional abundance of the one or more bacteria in the subject's gut microbiome is dissimilar to that of one or more subjects having demonstrated responsiveness to treatment, then characterizing the subject as likely to be non-responsive to treatment.

In an aspect, a disclosed method of reducing the degree and/or severity of a drug-resistant E. coli colonization in a subject can further comprise comparing the prevalence and/or proportional abundance of the one or more bacteria in the subject's gut microbiome to one or more standard levels, and if the prevalence and/or proportional abundance of the one or more bacteria in the subject's gut microbiome is similar to that of one or more subjects having demonstrated non-responsiveness to treatment, then characterizing the subject as likely to be non-responsive to treatment.

In an aspect, a disclosed method of reducing the degree and/or severity of a drug-resistant E. coli colonization in a subject can further comprise comparing the prevalence and/or proportional abundance of the one or more bacteria in the subject's gut microbiome to one or more standard levels, and if the prevalence and/or proportional abundance of the one or more bacteria in the subject's gut microbiome is dissimilar to that of one or more subjects having demonstrated non-responsiveness to treatment, then characterizing the subject as likely to be responsive to treatment.

In an aspect, a disclosed method of reducing the degree and/or severity of a drug-resistant E. coli colonization in a subject can further comprise generating a standard level or a standard range for prevalence and/or proportional abundance of one or more bacteria in a gut microbiome. In an aspect, a disclosed method of reducing the degree and/or severity of a drug-resistant E. coli colonization in a subject can further comprise generating a standard level or a standard range for prevalence and/or proportional abundance of one or more bacteria in a gut microbiome, wherein the standard level or the standard range represents a likelihood of non-responsiveness to treatment. In an aspect, a disclosed method of reducing the degree and/or severity of a drug-resistant E. coli colonization in a subject can further comprise generating a standard level or a standard range for prevalence and/or proportional abundance of one or more bacteria in a gut microbiome, wherein the standard level or the standard range represents a likelihood of responsiveness to treatment. In an aspect, a disclosed method of reducing the degree and/or severity of a drug-resistant E. coli colonization in a subject can further comprise treating the subject.

In an aspect, treating the subject can comprise administering a composition comprising (i) one or more agents that can reduce the intestinal carriage associated with the colonization of drug-resistant E. coli bacteria, (ii) one or more agents that can reduce the proportional abundance of one or more bacteria associated with the carriage and/or persistence of drug-resistant E. coli bacteria, (iii) one or more agents that can inhibit proliferation of Asaccharobacter, Bifidobacterium, Collinsella, Eggerthella, Gardnerella, Gordonibacter, or any combination thereof, (iv) one or more agents that can increase proliferation of Alistipes and/or Akkermansia.

In an aspect, a disclosed method of reducing the degree and/or severity of a drug-resistant E. coli colonization in a subject can further comprise comprising administering to the subject a therapeutically effective amount of one or more therapeutic agent. In an aspect, therapeutic agents can comprise any therapeutic agent disclosed herein. In an aspect, treating can comprise administering to the subject a therapeutically effective amount of one or more disclosed compositions. In an aspect, treating can comprise administering to the subject a therapeutically effective amount of one or more disclosed pharmaceutical formulations.

In an aspect, a disclosed sample can comprise a fecal sample. In an aspect, a disclosed fecal sample can be obtained using a fecal swab. In an aspect, the prevalence and/or the proportional abundance of the one or more bacterial in a sample can be generated by sequencing the 16S rRNA gene V3-V4 region. In an aspect, sequencing the 16S IRNA gene V3-V4 region can be performed according to the method described in as described in Fadrosh D W, et al. (2014) Microbiome. 2 (1): 6 by using MiSeq Reagent Kit v3 (600-cycle) (Illumina Inc., San Diego, CA). In an aspect, one or more negative extraction controls (NECs) can be included with each batch of extraction and can be sequenced to assess for cross contamination.

In an aspect, one or more no-template controls (NTCs) and one or more positive-template controls (PTCs) can be included to assess for cross-contamination and to verify PCR performance.

In an aspect, a disclosed method of reducing the degree and/or severity of a drug-resistant E. coli colonization in a subject can further comprise repeating the treating step one or more times.

In an aspect, a disclosed method of reducing the degree and/or severity of a drug-resistant E. coli colonization in a subject can further comprise repeating the treating step until the subject experiences an alleviation and/or a diminishment of one or more symptoms. In an aspect, the one or more symptoms can be associated with and/or due to the presence of one or more disclosed bacteria in the subject's gut microbiome. In an aspect, the presence of the one or more bacteria in the subject's gut microbiome can interfere with treating the subject for one or more diseases, infections, and/or disorder.

In an aspect, the presence of the one or more ST131 E. coli bacteria in the subject's gut microbiome can interfere with treating the subject for one or more diseases, infections, and/or disorder. In an aspect, the presence of the H30R1 E. coli bacteria and/or H30Rx E. coli bacteria in the subject's gut microbiome can interfere with treating the subject for one or more diseases, infections, and/or disorder. In an aspect, interfering can comprise disrupting and/or diminishing the efficacy of one or more treatments. In an aspect, interfering can comprise rendering the subject resistant to one or more antibiotics. In an aspect, interfering can comprise rendering the subject resistant to fluoroquinolone. In an aspect, the one or more E. coli bacteria can comprise sequence type 131 (ST131) and can be multi-drug resistant. In an aspect, the one or more ST131 E. coli bacteria can comprise a H30R subclone and can be multi-drug resistant. In an aspect, the one or more H30R subclones can comprise H30R1 or H30Rx and is multi-drug resistant. In an aspect, the one or more E. coli bacteria can be multi-drug resistant. In an aspect, the one or more E. coli bacteria can be fluoroquinolone resistant. In an aspect, the one or more E. coli bacteria can comprise sequence type 131 (ST131) E. coli bacteria and can be fluoroquinolone resistant. In an aspect, the one or more ST131 E. coli bacteria can comprise a H30R subclone and can be fluoroquinolone resistant. In an aspect, the one or more H30R subclones can comprise H30R1 or H30Rx and is fluoroquinolone resistant.

In an aspect, the one or more bacteria can comprise bacteria in the Actinobacter phylum, the Bacteroidetes phylum, the Firmicutes phylum, the Verruncomicrobia phylum, the Proteobacteria phylum, or any combination thereof. In an aspect, a higher proportional abundance of Actinobacteria can indicate that the subject is not likely to be responsive to antibiotic treatment. In an aspect, a lower proportional abundance of Actinobacteria can indicate that the subject is likely to be responsive to antibiotic treatment. In an aspect, a higher proportional abundance of Collinsella can indicate that the subject is not likely to be responsive to antibiotic treatment. In an aspect, a lower proportional abundance of Collinsella can indicate that the subject is likely to be responsive to antibiotic treatment. In an aspect, a lower proportional abundance of Verrucomicrobia can indicate that the subject is not likely to be responsive to antibiotic treatment. In an aspect, a higher proportional abundance of Verrucomicrobia can indicate that the subject is likely to be responsive to antibiotic treatment. In an aspect, a higher proportional abundance of Alistipes can indicate that the subject is likely to be responsive to antibiotic treatment. In an aspect, a lower proportional abundance of Alistipes can indicate that the subject is not likely to be responsive to antibiotic treatment. In an aspect, a higher proportional abundance of Akkermansia can indicate that the subject is likely to be responsive to antibiotic treatment. In an aspect, a lower proportional abundance of Akkermansia can indicate that the subject is not likely to be responsive to antibiotic treatment.

In an aspect, a higher proportional abundance of Actinobacteria can indicate a likelihood that the subject will be non-responsive to antibiotic treatment. In an aspect, a lower proportional abundance of Actinobacteria can indicate a likelihood that the subject will be responsive to antibiotic treatment. In an aspect, a higher proportional abundance of Collinsella can indicate a likelihood that the subject will be non-responsive to antibiotic treatment. In an aspect, a lower proportional abundance of Collinsella can indicate a likelihood that the subject will be responsive to antibiotic treatment. In an aspect, a lower proportional abundance of Verrucomicrobia can indicate a likelihood that the subject will be non-responsive to antibiotic treatment. In an aspect, a higher proportional abundance of Verrucomicrobia can indicate a likelihood that the subject will be responsive to antibiotic treatment. In an aspect, a higher proportional abundance of Alistipes can indicate a likelihood that the subject will be responsive to antibiotic treatment. In an aspect, a lower proportional abundance of Alistipes can indicate a likelihood that the subject will be non-responsive to antibiotic treatment. In an aspect, a higher proportional abundance of Akkermansia can indicate a likelihood that the subject will be responsive to antibiotic treatment. In an aspect, a lower proportional abundance of Akkermansia can indicate a likelihood that the subject will be non-responsive to antibiotic treatment.

In an aspect of a disclosed method of reducing the degree and/or severity of a drug-resistant E. coli colonization in a subject, a subject is likely to be responsive to antibiotic treatment when there is (i) a lower proportional abundance of Actinobacteria; (ii) a lower proportional abundance of Collinsella; (iii) a higher proportional abundance of Verrucomicrobia; (iv) a higher proportional abundance of Alistipes; (v) a higher proportional abundance of Akkermansia; or (vi) any combination thereof. In an aspect of a disclosed method of reducing the risk of developing a drug-resistant E. coli infection, a subject is not likely to be responsive to antibiotic treatment when there is (i) a higher proportional abundance of Actinobacteria; (ii) a higher proportional abundance of Collinsella; (iii) a lower proportional abundance of Verrucomicrobia; (iv) a lower proportional abundance of Alistipes; (v) a lower proportional abundance of Akkermansia; or (vi) any combination thereof.

In an aspect, a disclosed method of reducing the degree and/or severity of a drug-resistant E. coli colonization in a subject can further comprise further comprising measuring the prevalence and/or proportional abundance of one or more E. coli bacteria sequence type 131 (ST131). In an aspect, the one or more ST131 E. coli bacteria can comprise a H30R subclone and is fluoroquinolone resistant. In an aspect, the H30R subclone can comprise H30R1 or H30Rx and can be fluoroquinolone resistant.

In an aspect, a disclosed method of reducing the degree and/or severity of a drug-resistant E. coli colonization in a subject can further comprise administering to the subject one or more compositions that modulate the intestinal carriage and persistence of one or more ST131 E. coli bacteria. In an aspect, a disclosed method of reducing the degree and/or severity of a drug-resistant E. coli colonization in a subject can further comprise administering to the subject one or more compositions that modulate the intestinal carriage and persistence of H30R1 and/or H30Rx. In an aspect, a disclosed method of reducing the degree and/or severity of a drug-resistant E. coli colonization in a subject can further comprise administering to the subject one or more compositions that decrease and/or disrupt the intestinal carriage and persistence of one or more ST131 E. coli bacteria. In an aspect, a disclosed method of reducing the degree and/or severity of a drug-resistant E. coli colonization in a subject can further comprise administering to the subject one or more compositions that decrease and/or disrupt the intestinal carriage and persistence of H30R1 and/or H30Rx. In an aspect, a disclosed method of reducing the degree and/or severity of a drug-resistant E. coli colonization in a subject can further comprise administering to the subject one or more compositions that inhibit proliferation of H30R1 and/or H30Rx in the subject's gut microbiome and/or gastrointestinal tract.

In an aspect, a disclosed method of reducing the degree and/or severity of a drug-resistant E. coli colonization in a subject can further comprise administering to the subject one or more compositions that modulate the intestinal carriage and persistence of one or more bacteria. In an aspect, a disclosed method of reducing the degree and/or severity of a drug-resistant E. coli colonization in a subject can further comprise administering to the subject one or more compositions that decrease and/or disrupt the intestinal carriage and persistence of one or more bacteria. In an aspect, a disclosed method of reducing the degree and/or severity of a drug-resistant E. coli colonization in a subject can further comprise administering to the subject one or more compositions that inhibit proliferation of one or more bacteria in the subject's gut microbiome and/or gastrointestinal tract. In an aspect, a disclosed method of reducing the degree and/or severity of a drug-resistant E. coli colonization in a subject can further comprise administering to the subject one or more compositions that inhibit proliferation of one or more bacteria belonging to the Actinobacteria phylum in the subject's gut and/or gastrointestinal tract.

In an aspect, a disclosed method of reducing the degree and/or severity of a drug-resistant E. coli colonization in a subject can further comprise administering to the subject one or more compositions that inhibit proliferation of Asaccharobacter, Bifidobacterium, Collinsella, Eggerthella, Gardnerella, Gordonibacter, or any combination thereof in the subject's gut and/or gastrointestinal tract.

In an aspect, a disclosed method of reducing the degree and/or severity of a drug-resistant E. coli colonization in a subject can further comprise administering to the subject one or more compositions that inhibit proliferation of Collinsella in the subject's gut and/or gastrointestinal tract. In an aspect, a disclosed method of reducing the degree and/or severity of a drug-resistant E. coli colonization in a subject can further comprise administering to the subject one or more compositions that inhibit proliferation of Gardnerella and/or Gordonibacter in the subject's gut and/or gastrointestinal tract. In an aspect, a disclosed method of reducing the degree and/or severity of a drug-resistant E. coli colonization in a subject can further comprise administering to the subject one or more compositions that inhibit proliferation of one or more bacteria belonging to the Firmicutes phylum in the subject's gut and/or gastrointestinal tract. In an aspect, a disclosed method of reducing the degree and/or severity of a drug-resistant E. coli colonization in a subject can further comprise administering to the subject one or more compositions that inhibit proliferation of Enterococcus, Lactobacillus, Streptococcus, Weissella, or any combination thereof in the subject's gut and/or gastrointestinal tract. In an aspect, a disclosed method of reducing the degree and/or severity of a drug-resistant E. coli colonization in a subject can further comprise administering to the subject one or more compositions that inhibit proliferation of Lactobacillus and/or Streptococcus in the subject's gut and/or gastrointestinal tract.

In an aspect, a disclosed method of reducing the degree and/or severity of a drug-resistant E. coli colonization in a subject can further comprise administering to the subject one or more compositions that decrease and/or disrupt the intestinal carriage and persistence of one or more bacteria. In an aspect, a disclosed method of reducing the degree and/or severity of a drug-resistant E. coli colonization in a subject can further comprise administering to the subject one or more compositions that increase and/or stimulate proliferation of one or more bacteria in the subject's gut microbiome and/or gastrointestinal tract. In an aspect, a disclosed method of reducing the degree and/or severity of a drug-resistant E. coli colonization in a subject can further comprise administering to the subject one or more compositions that increase and/or stimulate proliferation of one or more bacteria belonging to the Bacteroidetes phylum in the subject's gut microbiome and/or gastrointestinal tract. In an aspect, a disclosed method of reducing the degree and/or severity of a drug-resistant E. coli colonization in a subject can further comprise administering to the subject one or more compositions that increase and/or stimulate proliferation of one or more bacteria belonging to the Verrucomicrobia phylum in the subject's gut microbiome and/or gastrointestinal tract. In an aspect, a disclosed method of reducing the degree and/or severity of a drug-resistant E. coli colonization in a subject can further comprise administering to the subject one or more compositions that increase and/or stimulate proliferation of Alistipes and/or Akkermansia the subject's gut microbiome and/or gastrointestinal tract. In an aspect, a lower proportional abundance of Verrucomicrobia can indicate that the subject is not likely to be responsive to antibiotic treatment. In an aspect, a higher proportional abundance of Verrucomicrobia can indicate that the subject is likely to be responsive to antibiotic treatment. In an aspect, a higher proportional abundance of Alistipes can indicate that the subject is likely to be responsive to antibiotic treatment. In an aspect, a lower proportional abundance of Alistipes can inhibit that the subject is not likely to be responsive to antibiotic treatment. In an aspect, a higher proportional abundance of Akkermansia can inhibit that the subject is likely to be responsive to antibiotic treatment. In an aspect, a lower proportional abundance of Akkermansia can inhibit that the subject is not likely to be responsive to antibiotic treatment.

In an aspect of a disclosed method of reducing the degree and/or severity of a drug-resistant E. coli colonization in a subject, a subject at risk can be any subject. In an aspect, an at-risk subject can comprise an adult, a young adult, a child, a toddler, or an infant. In an aspect, an at-risk subject can have cancer. In an aspect, an at-risk subject can be receiving chemotherapy. In an aspect, an at-risk subject can be receiving immunosuppressive therapy. In an aspect, an at-risk subject that does not have an infection and/or is not suspected of having an infection. In an aspect, following administration, the colonization of a pathogenic E. coli in the subject's intestinal tract and/or gut can be inhibited and/or prevented. In an aspect, a disclosed method of reducing the degree and/or severity of a drug-resistant E. coli colonization in a subject can further comprise promoting respiratory health in a subject. In an aspect, a disclosed biotherapeutic can inhibit and/or prevent the growth of one or more E. coli in one or more parts of the subject's respiratory system. In an aspect, a disclosed method of reducing the degree and/or severity of a drug-resistant E. coli colonization in a subject can further comprise characterizing the microbiome of a biological sample. In an aspect, a disclosed microbiome can comprise the gut microbiome, the gastrointestinal microbiome, or both. In an aspect, a disclosed method of reducing the degree and/or severity of a drug-resistant E. coli colonization in a subject can further comprise obtaining a biological sample from the subject. In an aspect, a disclosed biological sample can comprise a stool sample, a fecal swab, or any combination thereof.

In an aspect, a disclosed method of reducing the degree and/or severity of a drug-resistant E. coli colonization in a subject, characterizing the microbiome can comprise (i) collecting a biological sample from the subject; (ii) extracting nucleic acid from the subject's biological sample; and (iii) sequencing the extracted nucleic acid. In an aspect, sequencing the extracted nucleic acid can generate sequence data. In an aspect, characterizing the microbiome can further comprise analyzing the sequence data using taxonomic classification. In an aspect, using taxonomic classification can comprise PCR amplification. In an aspect, PCR amplification can comprise using primers targeting the 16S rRNA gene. In an aspect, PCR amplification can comprise primers targeting the V4 variable region of the 16S rRNA gene. In an aspect, disclosed primers can comprise a pair of primers.

In an aspect, a disclosed method of reducing the degree and/or severity of a drug-resistant E. coli colonization in a subject can further comprise identifying the one or more pathogenic bacteria. In an aspect, a disclosed method of reducing the risk of developing a E. coli infection can further comprise diagnosing the subject with an infection.

In an aspect of a disclosed method of reducing the degree and/or severity of a drug-resistant E. coli colonization in a subject, a disclosed therapeutically effective dose can comprise a total of at least 100 bacterial CFUs of the one or more probiotics, a total of at least 200 to at least 300 bacterial CFUs of the one or more probiotics, a total of at least 300 to at least 400 bacterial CFUs of the one or more probiotics, a total of at least 400 to at least 500 bacterial CFUs of the one or more probiotics, a total of at least 500 to at least 600 bacterial CFUs of the one or more probiotics, a total of at least 600 to at least 700 bacterial CFUs of the one or more probiotics, a total of at least 700 to at least 800 bacterial CFUs of the one or more probiotics, a total of at least 800 to at least 900 bacterial CFUs of the one or more probiotics, a total of at least 900 to at least 1000 bacterial CFUs of the one or more probiotics, or a total of at least 1000 bacterial CFUs of one or more disclosed probiotics (such as, for example, at least one strain from the bacterial genus Alistipes and/or at least one strain from the bacterial genus Akkermansia).

In an aspect of a disclosed pharmaceutical formulation, a disclosed therapeutically effective dose can comprise a total of at least 1000 to at least 2000 bacterial CFUs of the one or more probiotics, a total of at least 2000 to at least 3000 bacterial CFUs of the one or more probiotics, a total of at least 3000 to at least 4000 bacterial CFUs of the one or more probiotics, a total of at least 4000 to at least 5000 bacterial CFUs of the one or more probiotics, a total of at least 5000 to at least 6000 bacterial CFUs of the one or more probiotics, a total of at least 6000 to at least 7000 bacterial CFUs of the one or more probiotics, a total of at least 8000 to at least 9000 bacterial CFUs of the one or more probiotics, or a total of at least 10,000 bacterial CFUs of one or more disclosed probiotics (such as, for example, at least one strain from the bacterial genus Alistipes and/or at least one strain from the bacterial genus Akkermansia).

In an aspect of a disclosed method of reducing the risk of developing a E. coli infection, a disclosed therapeutically effective dose can comprise a total of at least 100 bacterial CFUs of at least one strain from the bacterial genus Alistipes and/or at least one strain from the bacterial genus Akkermansia, a total of at least 200 to at least 300 bacterial CFUs of at least one strain from the bacterial genus Alistipes and/or at least one strain from the bacterial genus Akkermansia, a total of at least 300 to at least 400 bacterial CFUs of at least one strain from the bacterial genus Alistipes and/or at least one strain from the bacterial genus Akkermansia, a total of at least 400 to at least 500 bacterial CFUs of at least one strain from the bacterial genus Alistipes and/or at least one strain from the bacterial genus Akkermansia, a total of at least 500 to at least 600 bacterial CFUs of at least one strain from the bacterial genus Alistipes and/or at least one strain from the bacterial genus Akkermansia, a total of at least 600 to at least 700 bacterial CFUs of at least one strain from the bacterial genus Alistipes and/or at least one strain from the bacterial genus Akkermansia, a total of at least 700 to at least 800 bacterial CFUs of at least one strain from the bacterial genus Alistipes and/or at least one strain from the bacterial genus Akkermansia, a total of at least 800 to at least 900 bacterial CFUs of at least one strain from the bacterial genus Alistipes and/or at least one strain from the bacterial genus Akkermansia, a total of at least 900 to at least 1000 bacterial CFUs of at least one strain from the bacterial genus Alistipes and/or at least one strain from the bacterial genus Akkermansia, or a total of at least 1000 bacterial CFUs of at least one strain from the bacterial genus Alistipes and/or at least one strain from the bacterial genus Akkermansia.

In an aspect of a disclosed method of reducing the degree and/or severity of a drug-resistant E. coli colonization in a subject, a disclosed therapeutically effective dose can comprise a total of at least 1000 to at least 2000 bacterial CFUs of at least one strain from the bacterial genus Alistipes and/or at least one strain from the bacterial genus Akkermansia, a total of at least 2000 to at least 3000 bacterial CFUs of at least one strain from the bacterial genus Alistipes and/or at least one strain from the bacterial genus Akkermansia, a total of at least 3000 to at least 4000 bacterial CFUs of at least one strain from the bacterial genus Alistipes and/or at least one strain from the bacterial genus Akkermansia, a total of at least 4000 to at least 5000 bacterial CFUs of at least one strain from the bacterial genus Alistipes and/or at least one strain from the bacterial genus Akkermansia, a total of at least 5000 to at least 6000 bacterial CFUs of at least one strain from the bacterial genus Alistipes and/or at least one strain from the bacterial genus Akkermansia, a total of at least 6000 to at least 7000 bacterial CFUs of at least one strain from the bacterial genus Alistipes and/or at least one strain from the bacterial genus Akkermansia, a total of at least 8000 to at least 9000 bacterial CFUs of at least one strain from the bacterial genus Alistipes and/or at least one strain from the bacterial genus Akkermansia, or a total of at least 10,000 bacterial CFUs of at least one strain from the bacterial genus Alistipes and/or at least one strain from the bacterial genus Akkermansia.

In an aspect of a disclosed method of reducing the degree and/or severity of a drug-resistant E. coli colonization in a subject, a disclosed therapeutically effective dose can comprise a total of at least 10⁴ to at least 10⁵ bacterial CFUs of the one or more probiotics, a total of at least 10⁵ to at least 10⁶ bacterial CFUs of the one or more probiotics, a total of at least 10⁶ to at least 10⁷ bacterial CFUs of the one or more probiotics, a total of at least 10⁷ to at least 10⁸ bacterial CFUs of the one or more probiotics, a total of at least 10⁸ to at least 10⁹ bacterial CFUs of the one or more probiotics, a total of at least 10⁹ to at least 10¹⁰ bacterial CFUs of the one or more probiotics, a total of at least 10¹⁰ to at least 10¹¹ bacterial CFUs of the one or more probiotics, a total of at least 10¹¹ to at least 10¹² bacterial CFUs of the one or more probiotics, a total of at least 10¹² bacterial CFUs of the one or more probiotics, or more than 10¹² bacterial CFUs of one or more disclosed probiotics (such as, for example, at least one strain from the bacterial genus Alistipes and/or at least one strain from the bacterial genus Akkermansia).

In an aspect of a disclosed method of reducing the degree and/or severity of a drug-resistant E. coli colonization in a subject, a disclosed therapeutically effective dose can comprise at least 10³, 10⁴, 10⁵, 10⁶, 10⁷, 10⁸, 10⁹, 10¹⁰, 10¹¹, or 10¹² CFUs of a disclosed probiotic or a consortium of disclosed probiotics (such as, for example, at least one strain from the bacterial genus Alistipes and/or at least one strain from the bacterial genus Akkermansia), preferably at least 1.2×10³, 1.4×10³, 2×10³, or 3×10³ CFUs of a disclosed probiotic or a consortium of disclosed probiotics, at least 5×10³, 5.02×10³, 5.04×10³, 5.2×10³, or 5.4×10³ CFUs of a disclosed probiotic or a consortium of disclosed probiotics, at least 1.02×10⁴, 1.04×10⁴, 1.2×10⁴, 1.4×10⁴, 1.5×10⁴, 2×10⁴, or 3×10⁴ CFUs of a disclosed probiotic or a consortium of disclosed probiotics, at least 5×10⁴, 5.02×10⁴, 5.04×10⁴, 5.2×10⁴, or 5.4×10⁴ CFUs of a disclosed probiotic or a consortium of disclosed probiotics, at least 1.02×10⁵, 1.04×10⁵, 1.2×10⁵, 1.4×10⁵, 1.5×10⁵, 2×10⁵, or 3×10⁵ CFUs of a disclosed probiotic or a consortium of disclosed probiotics, at least 1.02×10⁶, 1.04×10⁶, 1.2×10⁶, 1.4×10⁶, 2×10⁶, or 3×10⁶ CFUs of a disclosed probiotic or a consortium of disclosed probiotics, at least 1.02×10⁷, 1.04×10⁷, 1.2×10⁷, 1.4×10⁷, 2×10⁷, or 3×10⁷ CFUs of a disclosed probiotic or a consortium of disclosed probiotics, at least 1.02×10⁸, 1.04×10⁸, 1.2×10⁸, 1.4×10⁸, 2×10⁸, or 3×10⁸ CFUs of a disclosed probiotic or a consortium of disclosed probiotics, at least 1.02×10⁹, 1.04×10⁹, 1.2×10⁹, 1.4×10⁹, 2×10⁹, or 3×10⁹ CFUs of a disclosed probiotic or a consortium of disclosed probiotics, at least 10¹⁰ CFUs of a disclosed probiotic or a consortium of disclosed probiotics, at least 1.02×10¹⁰, 1.04×10¹⁰, 1.2×10¹⁰, 1.4×10¹⁰, 2×10¹⁰, or 3×10¹⁰ CFUs of a disclosed probiotic or a consortium of disclosed probiotics, at least 1.02×10¹¹, 1.04×10¹¹, 1.2×10¹¹, 1.4×10¹¹, 2×10¹¹, or 3×10¹¹ CFUs of a disclosed probiotic or a consortium of disclosed probiotics, or at least 1.02×10¹², 1.04×10¹², 1.2×10¹², 1.4×10¹², 2×10¹², or 3×10¹² CFUs of a disclosed probiotic or a consortium of disclosed probiotics per dose.

In an aspect, a disclosed therapeutically effective dose can comprise any amount or an unlimited amount of bacterial CFUs of the one or more probiotics.

In an aspect, a disclosed method of reducing the degree and/or severity of a drug-resistant E. coli colonization in a subject can further comprise treating the subject. In an aspect, treating the subject can comprise treating the subject's respiratory infection. In an aspect, treating the subject can comprise treating the subject's non-respiratory infection.

In an aspect, a disclosed method of reducing the degree and/or severity of a drug-resistant E. coli colonization in a subject can comprise further comprising administering to the subject a therapeutically effective amount of one or more anti-bacterial agents. Anti-bacterial agents and combinations of anti-bacterial agents are known to the art and discussed supr.

In an aspect, a disclosed method of reducing the degree and/or severity of a drug-resistant E. coli colonization in a subject can comprise further comprising administering to the subject a therapeutically effective amount of (i) one or more active agents, (ii) one or more biologically active agents, (iii) one or more pharmaceutically active agents, (iv) one or more immune-based therapeutic agents, (v) one or more clinically approved agents, or (vi) any combination thereof. In an aspect, a therapeutically effective amount of an active agent, a biologically active agent, a pharmaceutically active agent, an immune-based therapeutic active agent, a clinically approved agent, or an anti-bacterial agent can be about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, a disclosed method of reducing the degree and/or severity of a drug-resistant E. coli colonization in a subject can further comprise administering to the subject one or more therapeutic agents. In an aspect, a disclosed therapeutic agent can comprise a biologically active agent, a pharmaceutically active agent, an anti-bacterial agent, an anti-fungal agent, an anti-viral agent, a corticosteroid, an analgesic, an immunostimulant, an immune-based product, or any combination thereof. In an aspect of a disclosed method of promoting respiratory health, administering can comprise intranasal administration, oral administration, sublingual administration, or any combination thereof.

In an aspect, a disclosed method of reducing the degree and/or severity of a drug-resistant E. coli colonization in a subject, a subject can be a healthy subject, for example, a subject that does not have an infection and/or is not suspected of having an infection.

In an aspect, a subject can be an adult, a child, or an infant. In an aspect, a subject can be a neonate. In an aspect, a subject can be a premature infant. In an aspect, a subject can be immune-compromised. In an aspect, a subject can have diabetes or a chronic disease (e.g., heart disease, kidney disease, or liver disease). In an aspect, a subject can have HIV. In an aspect, a subject can have cancer or has had cancer. In an aspect, a subject can be the recipient of one or more solid organ transplants. In an aspect, a female subject can be pregnant. In an aspect, a subject can have or can be suspected of having a urinary tract infection (UTI). In an aspect, a subject can be in a high-risk environment (e.g., an acute care facility, a hospital, a longer-term acute care hospital, an in-patient facility, an in-patient rehabilitation facility, or a battle-zone or active war zone). In an aspect, a subject can be in the midst of a humanitarian crisis.

In an aspect, a subject can be diagnosed with an infection of one or more parts of the respiratory system. In an aspect, a disclosed therapeutically effective amount of a biotherapeutic can comprise an amount sufficient to inhibit and/or prevent the growth of one or more pathogenic bacteria in one or more parts of the subject's gastrointestinal system.

In an aspect, a disclosed method of reducing the degree and/or severity of a drug-resistant E. coli colonization in a subject can comprise a 10%, 20%, 30%, 40%, 50%, 60%, 70%, 80%, 90%, 100%, or any amount of inhibiting and/or preventing the growth and/or spread of one or more pathogenic bacteria (e.g., ST131 (H30R1 and/or H30Rx)) when compared to a control subject (such as, for example, a subject that has not received a disclosed biotherapeutic). In an aspect, a disclosed method of reducing the degree and/or severity of a drug-resistant E. coli colonization in a subject can comprise a 10-20%, 20-30%, 30-40%, 40-50%, 50-60%, 60-70%, 70-80%, 80-90%, or 90-100% or any amount of inhibiting and/or preventing the growth and/or spread of one or more pathogenic bacteria (e.g., ST131 (H30R1 and/or H30Rx)) when compared to a control subject (such as, for example, a subject that has not received a disclosed biotherapeutic).

In an aspect, a disclosed method of reducing the degree and/or severity of a drug-resistant E. coli colonization in a subject can comprise a 10%, 20%, 30%, 40%, 50%, 60%, 70%, 80%, 90%, 100%, or any amount of inhibiting and/or preventing the adherence and/or colonization of one or more pathogenic bacteria (e.g., ST131 (H30R1 and/or H30Rx)) when compared to a control subject (such as, for example, a subject that has not received a disclosed biotherapeutic). In an aspect, a disclosed method of reducing the degree and/or severity of a drug-resistant E. coli colonization in a subject can comprise a 10-20%, 20-30%, 30-40%, 40-50%, 50-60%, 60-70%, 70-80%, 80-90%, or 90-100% or any amount of inhibiting and/or preventing the adherence and/or colonization of one or more pathogenic bacteria (e.g., ST131 (H30R1 and/or H30Rx)) when compared to a control subject (such as, for example, a subject that has not received a disclosed biotherapeutic).

In an aspect, a disclosed method of reducing the degree and/or severity of a drug-resistant E. coli colonization in a subject can comprise a 10%, 20%, 30%, 40%, 50%, 60%, 70%, 80%, 90%, 100%, or any amount of modulating the endocytosis or paracellular migration of one or more pathogenic bacteria (e.g., ST131 E. coli (i.e., H30R1 and/or H30Rx)) when compared to a control subject (such as, for example, a subject that has not received a disclosed biotherapeutic). In an aspect, a disclosed method of reducing the degree and/or severity of a drug-resistant E. coli colonization in a subject can comprise a 10-20%, 20-30%, 30-40%, 40-50%, 50-60%, 60-70%, 70-80%, 80-90%, or 90-100% or any amount of modulating the endocytosis or paracellular migration of one or more pathogenic bacteria (e.g., ST131 E. coli (i.e., H30R1 and/or H30Rx)) when compared to a control subject (such as, for example, a subject that has not received a disclosed biotherapeutic).

In an aspect, a disclosed method of reducing the degree and/or severity of a drug-resistant E. coli colonization in a subject can comprise a 10%, 20%, 30%, 40%, 50%, 60%, 70%, 80%, 90%, 100%, or any amount of inhibiting and/or preventing the spread of one or more pathogenic bacteria (e.g., ST131 E. coli (i.e., H30R1 and/or H30Rx)) by competitive adherence and/or niche occupancy when compared to a control subject (such as, for example, a subject that has not received a disclosed biotherapeutic). In an aspect, a disclosed method of reducing the degree and/or severity of a drug-resistant E. coli colonization in a subject can comprise a 10-20%, 20-30%, 30-40%, 40-50%, 50-60%, 60-70%, 70-80%, 80-90%, or 90-100% or any amount of inhibiting and/or preventing the spread of one or more pathogenic bacteria (e.g., ST131 E. coli (i.e., H30R1 and/or H30Rx)) by competitive adherence and/or niche occupancy when compared to a control subject (such as, for example, a subject that has not received a disclosed biotherapeutic).

In an aspect, a disclosed method of reducing the degree and/or severity of a drug-resistant E. coli colonization in a subject can comprise repeating the administering of one or more disclosed biotherapeutics, one or more disclosed secreted factors, a disclosed consortium of probiotics, one or more disclosed probiotics, or any combination thereof. For example, in an aspect, repeating the administering can comprise administering one or more times daily (e.g., 1, 2, 3, or 4 times). In an aspect, repeating the administering can comprise administering on several consecutive days (e.g., for 1, 2, 3, 4, 5, 6, 7, 8, 9, or 10 days, or more than 10 days), weeks (e.g., for 1, 2, 3, 4, 5, 6, 7, 8, 9, or 10 weeks, or more than 10 weeks), or months (e.g., for 1, 2, 3, 4, 5, or 6 months, or more than 6 months). In an aspect, a disclosed biotherapeutic, a disclosed secreted factor, a disclosed consortium of probiotics, a disclosed probiotic, or any combination thereof can be administered 1-3 times per day for about 7 to 21 days.

In an aspect, a disclosed method of reducing the degree and/or severity of a drug-resistant E. coli colonization in a subject can comprise monitoring the subject. In an aspect, monitoring the subject can comprise monitoring the subject for the development of adverse effects. In an aspect, in the absence of adverse effects, a disclosed method of reducing the risk of developing a pathogenic E. coli infection can comprise continuing to treat the subject. In an aspect, continuing to treat the subject can comprise continuing to administer to the subject one or more disclosed biotherapeutics, one or more disclosed secreted factors, a disclosed consortium of probiotics, one or more disclosed probiotics, or any combination thereof. In an aspect, in the presence of adverse effects, a disclosed method of reducing the risk of developing a pathogenic E. coli infection can comprise modifying one or more steps of the method. In an aspect, modifying one or more steps of a disclosed method can comprise modifying the administering step. In an aspect, modifying the administering step can comprise changing the amount of one or more disclosed biotherapeutics, one or more disclosed secreted factors, a disclosed consortium of probiotics, one or more disclosed probiotics, or any combination thereof administered to the subject, changing the frequency of administration of one or more disclosed biotherapeutics, one or more disclosed secreted factors, a disclosed consortium of probiotics, one or more disclosed probiotics, or any combination thereof, changing the duration of administration of one or more disclosed biotherapeutics, one or more disclosed secreted factors, a disclosed consortium of probiotics, one or more disclosed probiotics, or any combination thereof, changing the route of administration of one or more disclosed biotherapeutics, one or more disclosed secreted factors, a disclosed consortium of probiotics, one or more disclosed probiotics, or any combination thereof, or any combination of disclosed changes.

In an aspect, a disclosed method of reducing the degree and/or severity of a drug-resistant E. coli colonization in a subject can comprise manipulating the microbiome to inhibit and/or prevent infection caused by one or more pathogenic bacteria, such as an infection caused by ST131 E. coli (i.e., H30R1 and/or H30Rx). In an aspect, a disclosed method of reducing the degree and/or severity of a drug-resistant E. coli colonization in a subject can comprise manipulating the microbiome to decrease and/or minimize the risk of infection caused by one or more pathogenic bacteria, such as an infection caused by ST131 E. coli (i.e., H30R1 and/or H30Rx).

In an aspect, a disclosed method of reducing the degree and/or severity of a drug-resistant E. coli colonization in a subject can comprise subjecting the subject to one or more invasive or non-invasive diagnostic assessments. Diagnostic assessments are known to the art. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination thereof. In an aspect, a disclosed invasive diagnostic assessment can comprise a tissue biopsy or exploratory surgery. In an aspect of a disclosed method of reducing the degree and/or severity of a drug-resistant E. coli colonization in a subject, techniques to monitor, measure, and/or assess the restoring one or more aspects of respiratory health can comprise qualitative (or subjective) means as well as quantitative (or objective) means. These means are known to the skilled person. For example, representative regulated variables and sensors relating to systemic homeostasis are discussed supra.

In an aspect of a disclosed method of reducing the degree and/or severity of a drug-resistant E. coli colonization in a subject, a disclosed composition, a disclosed pharmaceutical formulation, a disclosed biotherapeutic, a disclosed bacteria belonging to Alistipes genus and/or a disclosed bacteria belonging to Akkermansia, a disclosed secreted factor, a disclosed consortium of probiotics, a disclosed probiotic (e.g., Alistipes and/or Akkermansia), or any combination thereof can be administered in the absence of identifying the one or more pathogenic bacteria and/or in the absence of characterizing the microbiome. In an aspect of a disclosed method, a disclosed composition, a disclosed pharmaceutical formulation, a disclosed biotherapeutic, a disclosed bacteria belonging to Alistipes genus and/or a disclosed bacteria belonging to Akkermansia, a disclosed secreted factor, a disclosed consortium of probiotics, a disclosed probiotic (e.g., Alistipes and/or Akkermansia), or any combination thereof can be administered on demand.

In an aspect of a disclosed method of reducing the degree and/or severity of a drug-resistant E. coli colonization in a subject, a disclosed biotherapeutic can be formulated for administration as a pharmaceutical formulation. In an aspect, a disclosed pharmaceutical formulation comprising a probiotic, a consortium of probiotics, factors secreted from a probiotic, factors secreted from a consortium of probiotics, or any combination thereof, and a pharmaceutically acceptable carrier and/or excipient can be used in any disclosed method of reducing the risk of developing a pathogenic E. coli infection. Any pharmaceutical formulation disclosed herein can be used in a disclosed method of reducing the risk of developing a pathogenic ST131 E. coli infection (i.e., H30R1 and/or H30Rx).

In an aspect, a disclosed method can further (i) reduce the risk of developing a colonization of drug-resistant E. coli, (ii) reduce the degree and/or severity of a drug-resistant E. coli infection, (iii) reduce the degree and/or severity of a drug-resistant E. coli colonization, (iv) reduce the degree and/or severity of a drug-resistant E. coli colonization, or (v) any combination thereof.

In an aspect, a disclosed method can further comprise administering to the subject one or more broad-spectrum antibiotic or antibiotic agents.

E. Methods of Increasing Resistance to a Drug-Resistant E. coli Infection

Disclosed herein is a method of increasing resistance to a drug-resistant E. coli infection and/or colonization in a subject, the method comprising administering to a subject in need thereof a therapeutically effective amount of a composition comprising one or more bacteria belonging to the Alistipes genus and/or Akkermansia genus, wherein following the administering step the composition reduces the intestinal carriage and/or persistence of one or more E. coli bacteria, thereby increasing the subject's resistance to an infection and/or colonization of a drug-resistance E. coli. Disclosed herein is a method of increasing resistance to a drug-resistant E. coli infection and/or colonization in a subject, the method comprising administering to a subject in need thereof a therapeutically effective amount of a composition comprising one or more bacteria belonging to the Alistipes genus and/or Akkermansia genus, wherein following the administering step the composition reduces the intestinal carriage and/or persistence of one or more E. coli bacteria, thereby increasing the subject's resistance to an infection and/or colonization of a drug-resistance E. coli.

In an aspect, one or more disclosed E. coli bacteria can comprise sequence type 131 (ST131). In an aspect, one or more disclosed ST131 E. coli bacteria can comprise a H30R subclone. In an aspect, a disclosed H30R subclone can comprise H30R1 or H30Rx. In an aspect, one or more disclosed E. coli bacteria can be multi-drug resistant. In an aspect, one or more disclosed E. coli bacteria are fluoroquinolone resistant. In an aspect, one or more disclosed E. coli bacteria can comprise sequence type 131 (ST131) and can be multi-drug resistant. In an aspect, one or more disclosed ST131 E. coli bacteria can comprise a H30R subclone and can be multi-drug resistant. In an aspect, one or more disclosed H30R subclones can comprise H30R1 or H30Rx and can be multi-drug resistant. In an aspect, one or more disclosed E. coli bacteria can comprise sequence type 131 (ST131) E. coli bacteria and can be fluoroquinolone resistant. In an aspect, one or more disclosed ST131 E. coli bacteria can comprise a H30R subclone and can be fluoroquinolone resistant. In an aspect, one or more disclosed H30R subclones can comprise H30R1 or H30Rx and can be fluoroquinolone resistant.

In an aspect, a disclosed composition can comprise both one or more bacteria belonging to the Alistipes genus and one or more bacteria belonging to the Akkermansia genus. In an aspect, a disclosed composition can comprise one or more bacteria belonging to the Alistipes genus. In an aspect, a disclosed composition can comprise one or more bacteria belonging to an addition genus. In an aspect, a disclosed composition can comprise one or more bacterial belonging to the Alistipes genus and one or more bacteria belonging to the Akkermansia genus. In an aspect, a disclosed composition can comprise one or more bacteria belonging to the Alistipes genus. In an aspect, the Alistipes genus can comprise Alistipes finegoldii, Alistipes putredinis, Alistipes onderdonkii, Alistipes shahii, Alistipes indistinctus, Alistipes senegalensis, Alistipes timonensis, Alistipes obesi, Alistipes ihumii, Alistipes inops, Alistipes megaguti, Alistipes provencensis, Alistipes massiliensis, any combination thereof. In an aspect, a disclosed composition can comprise one or more bacterial species belonging to the Verrucomicrobia phylum. In an aspect, a disclosed composition can comprise one or more bacterial belonging to the Alistipes genus and one or more bacteria belonging to the Akkermansia genus.

In an aspect, a disclosed composition can comprise one or more agents that can reduce the intestinal carriage associated with the colonization of drug-resistant E. coli bacteria, thereby reducing the risk of an infection. In an aspect, a disclosed composition can comprise one or more agents that can reduce the proportional abundance of one or more bacteria associated with the carriage and/or persistence of drug-resistant E. coli bacteria, thereby reducing the degree and/or severity of colonization by a drug-resistance E. coli. In an aspect, a disclosed composition can comprise one or more agents that can inhibit proliferation of Asaccharobacter, Bifidobacterium, Collinsella, Eggerthella, Gardnerella, Gordonibacter, or any combination thereof in the gut microbiome of the subject. In an aspect, a disclosed composition can comprise one or more agents that can increase proliferation of Alistipes and/or Akkermansia.

In an aspect, a disclosed composition can comprise (i) one or more agents that can reduce the intestinal carriage associated with the colonization of drug-resistant E. coli bacteria, (ii) one or more agents that can reduce the proportional abundance of one or more bacteria associated with the carriage and/or persistence of drug-resistant E. coli bacteria, (iii) one or more agents that can inhibit proliferation of Asaccharobacter, Bifidobacterium, Collinsella, Eggerthella, Gardnerella, Gordonibacter, or any combination thereof in the gut microbiome of the subject, (iv) one or more agents that can increase proliferation of Alistipes and/or Akkermansia, or (v) any combination thereof.

In an aspect, measuring the prevalence and/or proportional abundance of one or more bacteria in the subject's gut microbiome can inform a clinician's treatment decisions.

In an aspect, a disclosed method can further comprise administering to the subject one or more bacteriophages capable of killing E. coli. In an aspect, a disclosed E. coli-killing bacteriophage can comprise BP539, BP700, BP753, BP814, BP953, BP954, BP970, BP1002, BP1151, BP1155, BP1168, BP1176, BP1197, BP1226, BP1229, or any combination thereof. In an aspect, a disclosed E. coli-killing bacteriophage can comprise MV36.2, JJ2050.2, C3, C19T, JJ6.1, or any combination thereof.

In an aspect, relative species abundance is a component of biodiversity. In an aspect, relative species abundance is a measure of how common or rare a species is relative to other species in a defined location or community. In an aspect, relative species abundance is a measure of how common or rare a species is relative to other species in a subject's gut microbiome. In an aspect, relative species abundance is a measure of how common or rare a species is relative to other species in a subject's gut microbiome prior to a disclosed administering step. In an aspect, relative species abundance is a measure of how common or rare a species is relative to other species in a subject's gut microbiome following a disclosed administering step. In an aspect, relative abundance is the percent composition of an organism of a particular kind relative to the total number of organisms in the area. In an aspect, relative abundance is the percent composition of an organism of a particular kind relative to the total number of organisms in a subject's gut microbiome. In an aspect, relative abundance is the percent composition of an organism of a particular kind relative to the total number of organisms in a subject's gut microbiome prior to a disclosed administering step. In an aspect, relative abundance is the percent composition of an organism of a particular kind relative to the total number of organisms in a subject's gut microbiome following a disclosed administering step.

In an aspect, abundance can be the number of individuals of a given species in a region. In an aspect, incidence can be the number of samples containing at least one of a given species in a census. In an aspect, sampling scope can be the area, interval of time, and taxonomic grouping over which sampling takes place. In an aspect, and in the context of community sampling, spatial heterogeneity can be variation in species capture probabilities across space. In an aspect, spatial heterogeneity can be due to habitat variation, intraspecific clumping, interspecific association, other factors, or any combination thereof. In an aspect, species abundance distribution can be the number of species found in each interval of abundance in a community.

In an aspect, species accumulation curve can be a plot of the total number of species observed in a census against some measure of cumulative sampling effort. In an aspect, species accumulation can be any of a number of measures concerning the number of species in an area and/or the distribution of their abundances. In an aspect, species richness can be the number of species present in a region.

In an aspect, and in the context of community sampling, temporal heterogeneity can be variation in species capture probabilities over time. In an aspect, temporal heterogeneity can arise from temporal environmental variation, migration, speciation or other factors. In an aspect, bacterial diversity can comprise two components: (i) species richness (i.e., the number of species in the community), and (ii) evenness (i.e., the fact that some species in the community are common and others are rare). In an aspect, an important component of most diversity indices is a value expressing the relative proportion of the species in the community (pi). In an aspect, this value can be calculated as the absolute (raw) abundance or dominance of the species (i) in the community, divided by the sum of all species abundances/dominances in that community. In an aspect, the sum of relative abundances for all species in the community equals to unity.

In an aspect of a disclosed method of increasing resistance to a drug-resistant E. coli infection and/or colonization in a subject, prior to the administering step the gut microbiome of the subject can comprise a higher proportional abundance of one or more bacteria belonging to the Actinobacteria phylum than that of a control subject. In an aspect, Actinobacteria phylum can comprise Asaccharobacter, Bifidobacterium, Collinsella, Eggerthella, Gardnerella, Gordonibacter, or any combination thereof. In an aspect of a disclosed method of increasing resistance to a drug-resistant E. coli infection and/or colonization in a subject, prior to the administering step the gut microbiome of the subject can comprise a higher proportional abundance of Collinsella than that of a control subject. In an aspect of a disclosed method of increasing resistance to a drug-resistant E. coli infection and/or colonization in a subject, prior to the administering step the gut microbiome of the subject can comprise a higher proportional abundance of Gardnerella and/or Gordonibacter than that of a control subject. In an aspect of a disclosed method of increasing resistance to a drug-resistant E. coli infection and/or colonization in a subject, prior to the administering step the gut microbiome of the subject can comprise a higher proportional abundance of one or more bacteria belonging to the Firmicutes phylum than that of a control subject. In an aspect, Firmicutes phylum can comprise Enterococcus, Lactobacillus, Streptococcus, Weissella, or any combination thereof. In an aspect of a disclosed method of increasing resistance to a drug-resistant E. coli infection and/or colonization in a subject, prior to the administering step the gut microbiome of the subject can comprise a higher proportional abundance of Lactobacillus and/or Streptococcus than that of a control subject.

In an aspect of a disclosed method of increasing resistance to a drug-resistant E. coli infection and/or colonization in a subject, prior to the administering step the gut microbiome of the subject can comprise a lower proportional abundance of one or more bacteria belonging to the Bacteroidetes phylum than that of a control subject. In an aspect, Bacteroidetes phylum can comprise Alistipes and/or Bacteroides. In an aspect, Bacteroides can comprise B. dorei, B. uniformis, B. eggerthii, B. stercoris, B. fragilis, B. caccae, B. intestinalis, B. ovatus, B. xylanisolvens, B. splanchnicus, or any combination thereof. In an aspect, Alistipes can comprise Alistipes finegoldii, Alistipes putredinis, Alistipes onderdonkii, Alistipes shahii, Alistipes indistinctus, Alistipes senegalensis, Alistipes timonensis, Alistipes obesi, Alistipes ihumii, Alistipes inops, Alistipes megaguti, Alistipes provencensis, Alistipes massiliensis, any combination thereof.

In an aspect, prior to the administering step the gut microbiome of the subject can comprise a lower proportional abundance of Bacteroides than that of a control subject. In an aspect, prior to the administering step the gut microbiome of the subject can comprise a lower proportional abundance of Alistipes than that of a control subject. In an aspect, prior to the administering step the gut microbiome of the subject can comprise a lower proportional abundance of one or more bacteria belonging to Verrucomicrobia phylum than that of a control subject. In an aspect, Verrucomichrobia phylum can comprise family Akkermansiaceae and family Verrucomicrobiaceae. In an aspect, Akkermansiaceae can comprise Akkermansia. In an aspect of a disclosed method of increasing resistance to a drug-resistant E. coli infection and/or colonization in a subject, wherein prior to the administering step the gut microbiome of the subject can comprise a lower proportional abundance of Akkermansia than that of a control subject.

In an aspect of a disclosed method of increasing resistance to a drug-resistant E. coli infection and/or colonization in a subject, wherein following the administering step, the proportional abundance of one or more E. coli sequence type 131 (ST131) bacteria in the gut microbiome of the subject is decreased. In an aspect of a disclosed method of increasing resistance to a drug-resistant E. coli infection and/or colonization in a subject, wherein, following the administering step, the proportional abundance of ST131-H30R1 in the gut microbiome of the subject is decreased. In an aspect of a disclosed method of increasing resistance to a drug-resistant E. coli infection and/or colonization in a subject, wherein, following the administering step, the proportional abundance of ST131-H30Rx in the gut microbiome of the subject is decreased. In an aspect of a disclosed method of increasing resistance to a drug-resistant E. coli infection and/or colonization in a subject, wherein, following the administering step, the proportional abundance of ST131-H30R1 and ST131-H30Rx in the gut microbiome of the subject is decreased. In an aspect of a disclosed method of increasing resistance to a drug-resistant E. coli infection and/or colonization in a subject, wherein, following the administering step, the level of and/or the amount of ST131-H30R1 and/or the level of and/or the amount of ST131-H30Rx in the gut microbiome of the subject is decreased from the level and/or the amount prior to the administering step.

In an aspect of a disclosed method of increasing resistance to a drug-resistant E. coli infection and/or colonization in a subject, wherein, following the administering step, the proportional abundance of one or more bacteria belonging to Actinobacteria phylum in the gut microbiome of the subject is decreased. In an aspect of a disclosed method of increasing resistance to a drug-resistant E. coli infection and/or colonization in a subject, wherein, following the administering step, the proportional abundance of Asaccharobacter, Bifidobacterium, Collinsella, Eggerthella, Gardnerella, Gordonibacter, or any combination thereof in the gut microbiome of the subject is decreased. In an aspect of a disclosed method of increasing resistance to a drug-resistant E. coli infection and/or colonization in a subject, wherein, following the administering step, the proportional abundance of Collinsella in the gut microbiome of the subject is decreased. In an aspect of a disclosed method of increasing resistance to a drug-resistant E. coli infection and/or colonization in a subject, following the administering step, the proportional abundance of Gardnerella and/or Gordonibacter in the gut microbiome of the subject is decreased. In an aspect of a disclosed method of increasing resistance to a drug-resistant E. coli infection and/or colonization in a subject, wherein, following the administering step, the proportional abundance of one or more bacteria belonging to Firmicutes phylum in the gut microbiome of the subject is decreased.

In an aspect of a disclosed method of increasing resistance to a drug-resistant E. coli infection and/or colonization in a subject, wherein, following the administering step, the proportional abundance of Enterococcus, Lactobacillus, Streptococcus, Weissella, or any combination thereof in the gut microbiome of the subject is decreased.

In an aspect of a disclosed method of increasing resistance to a drug-resistant E. coli infection and/or colonization in a subject, wherein, following the administering step, the proportional abundance of Lactobacillus and/or Streptococcus in the gut microbiome of the subject is decreased.

In an aspect of a disclosed method of increasing resistance to a drug-resistant E. coli infection and/or colonization in a subject, wherein, following the administering step, the level of and/or the amount of ST131-H30R1 and/or ST131-H30Rx in the gut microbiome of the subject is decreased from the level and/or the amount prior to the administering step. In an aspect of a disclosed method of increasing resistance to a drug-resistant E. coli infection and/or colonization in a subject, wherein, following the administering step, the level of and/or the amount of one or more bacteria belonging to the Actinobacteria phylum in the gut microbiome of the subject is decreased from the level and/or the amount prior to the administering step.

In an aspect of a disclosed method of increasing resistance to a drug-resistant E. coli infection and/or colonization in a subject, wherein, following the administering step, the level of and/or the amount of one or more bacteria belonging to the Actinobacteria phylum in the gut microbiome of the subject is decreased from the level and/or the amount prior to the administering step. In an aspect of a disclosed method of increasing resistance to a drug-resistant E. coli infection and/or colonization in a subject, wherein, following the administering step, the level of and/or the amount of Asaccharobacter, Bifidobacterium, Collinsella, Eggerthella, Gardnerella, Gordonibacter, or any combination thereof in the gut microbiome of the subject is decreased from the level and/or the amount prior to the administering step. In an aspect of a disclosed method of increasing resistance to a drug-resistant E. coli infection and/or colonization in a subject, wherein, following the administering step, the level of and/or the amount Collinsella in the gut microbiome of the subject is decreased from the level and/or the amount prior to the administering step.

In an aspect of a disclosed method of increasing resistance to a drug-resistant E. coli infection and/or colonization in a subject, wherein, following the administering step, the level of and/or the amount of Gardnerella and/or Gordonibacter in the gut microbiome of the subject is decreased from the level and/or the amount prior to the administering step.

In an aspect of a disclosed method of increasing resistance to a drug-resistant E. coli infection and/or colonization in a subject, wherein, following the administering step, the level of and/or the amount of one or more bacteria belonging to the Firmicutes phylum in the gut microbiome of the subject is decreased from the level and/or the amount prior to the administering step. I In an aspect of a disclosed method of increasing resistance to a drug-resistant E. coli infection and/or colonization in a subject, wherein, following the administering step, the level of and/or the amount of Enterococcus, Lactobacillus, Streptococcus, Weissella, or any combination thereof in the gut microbiome of the subject is decreased from the level and/or the amount prior to the administering step. In an aspect of a disclosed method of increasing resistance to a drug-resistant E. coli infection and/or colonization in a subject, wherein, following the administering step, the level of and/or the amount of Lactobacillus and/or Streptococcus in the gut microbiome of the subject is decreased from the level and/or the amount prior to the administering step.

In an aspect, gastrointestinal administration can comprise oral, nasogastric, or rectal administration. In an aspect, a disclosed composition can comprise a liquid, a suspension, a dried powder, a tablet, a capsule, or a food product.

In an aspect, a disclosed method of increasing resistance to a drug-resistant E. coli infection and/or colonization in a subject can further comprise administering to the subject an anti-toxin antibody, an herbal remedy, a probiotic bacteria, a probiotic yeast, or any combination thereof. In an aspect, a disclosed method of increasing resistance to a drug-resistant E. coli infection and/or colonization in a subject can further comprise administering to the subject one or more antibiotics, anti-toxin antibodies, herbal remedies, probiotic bacteria, probiotic yeast, or any combination thereof. In an aspect, a disclosed method of increasing resistance to a drug-resistant E. coli infection and/or colonization in a subject can further comprise administering to the subject antibiotic, an anti-toxin antibody, an herbal remedy, a probiotic bacteria, a probiotic yeast, or any combination thereof.

In an aspect of a disclosed method of increasing resistance to a drug-resistant E. coli infection and/or colonization in a subject, the therapeutically effective amount can ameliorate at least one symptom of an E. coli infection. In an aspect, a disclosed symptom of an E. coli infection can comprise abdominal tenderness, abdominal pain, abdominal cramping, sepsis, endocarditis, meningitis, headache, stiff neck, confusion, back pain, pneumonia, fever, chills, diarrhea, urinary tract infection, endocarditis, elevated white blood cell count, and decreased serum albumin.

In an aspect, a disclosed therapeutically effective amount can inhibit proliferation of one or more E. coli bacteria in the gastrointestinal tract of the subject. In an aspect, a disclosed therapeutically effective amount can inhibit proliferation of one or more E. coli bacteria in the gut microbiome of the subject.

In an aspect, a disclosed therapeutically effective amount can inhibit proliferation of E. coli bacteria can comprise sequence type 131 (ST131) in the gastrointestinal tract of the subject. In an aspect, a disclosed therapeutically effective amount can inhibit proliferation of E. coli bacteria can comprise sequence type 131 (ST131) in the gut microbiome of the subject. In an aspect, a disclosed therapeutically effective amount can inhibit proliferation of H30R in the gastrointestinal tract of the subject. In an aspect, a disclosed therapeutically effective amount can inhibit proliferation of H30R in the gut microbiome of the subject.

In an aspect, a disclosed therapeutically effective amount can inhibit proliferation of H30Rx in the gastrointestinal tract of the subject. In an aspect, a disclosed therapeutically effective amount can inhibit proliferation of H30Rx in the gut microbiome of the subject.

In an aspect, a disclosed therapeutically effective amount can inhibit proliferation of one or more bacteria belonging to Actinobacteria phylum in the gastrointestinal tract of the subject. In an aspect, a disclosed therapeutically effective amount can inhibit proliferation of one or more bacteria belonging to Actinobacteria phylum in the gut microbiome of the subject.

In an aspect, a disclosed therapeutically effective amount can inhibit proliferation of Asaccharobacter, Bifidobacterium, Collinsella, Eggerthella, Gardnerella, Gordonibacter, or any combination thereof in the gastrointestinal tract of the subject. In an aspect, a disclosed therapeutically effective amount can inhibit proliferation of Asaccharobacter, Bifidobacterium, Collinsella, Eggerthella, Gardnerella, Gordonibacter, or any combination thereof in the gut microbiome of the subject.

In an aspect, a disclosed therapeutically effective amount can inhibit proliferation of Collinsella in the gastrointestinal tract of the subject. In an aspect, a disclosed therapeutically effective amount can inhibit proliferation of Collinsella in the gut microbiome of the subject.

In an aspect, a disclosed therapeutically effective amount can inhibit proliferation of Gardnerella and/or Gordonibacter in the gastrointestinal tract of the subject. In an aspect, a disclosed therapeutically effective amount can inhibit proliferation of Gardnerella and/or Gordonibacter in the gut microbiome of the subject.

In an aspect, a disclosed therapeutically effective amount can inhibit proliferation of one or more bacteria belonging to Firmicutes phylum in the gastrointestinal tract of the subject. In an aspect, a disclosed therapeutically effective amount can inhibit proliferation of one or more bacteria belonging to Firmicutes phylum in the gut microbiome of the subject.

In an aspect, a disclosed therapeutically effective amount can inhibit proliferation of Enterococcus, Lactobacillus, Streptococcus, Weissella, or any combination thereof in the gastrointestinal tract of the subject. In an aspect, a disclosed therapeutically effective amount can inhibit proliferation of Enterococcus, Lactobacillus, Streptococcus, Weissella, or any combination thereof in the gut microbiome of the subject.

In an aspect, a disclosed therapeutically effective amount can inhibit proliferation of Lactobacillus and/or Streptococcus in the gastrointestinal tract of the subject. In an aspect, a disclosed therapeutically effective amount can inhibit proliferation of Lactobacillus and/or Streptococcus in the gut microbiome of the subject.

In an aspect of a disclosed method of increasing resistance to a drug-resistant E. coli infection and/or colonization in a subject, wherein following the administering step, the subject can be now susceptible and/or more susceptible to fluoroquinolone. In an aspect of a disclosed method of increasing resistance to a drug-resistant E. coli infection and/or colonization in a subject, wherein following the administering step, the subject can be now susceptible and/or more susceptible to antibiotic treatment. In an aspect of a disclosed method of increasing resistance to a drug-resistant E. coli infection and/or colonization in a subject, wherein following the administering step, the subject can be now susceptible and/or more susceptible to a treatment regimen comprising one or more antibiotic agents.

In an aspect, a disclosed method of increasing resistance to a drug-resistant E. coli infection and/or colonization in a subject can further comprise measuring the prevalence and/or proportional abundance of one or more Escherichia coli (E. coli) bacteria. In an aspect, E. coli bacteria can comprise sequence type 131 (ST131). In an aspect, ST131 E. coli bacteria can comprise a H30R subclone. In an aspect, a disclosed H30R subclone can comprise H30R1 or H30Rx. In an aspect, a disclosed E. coli bacteria can comprise sequence type 131 (ST131) and can be multi-drug resistant. In an aspect, a disclosed ST131 E. coli bacteria can comprise a H30R subclone and can be multi-drug resistant. In an aspect, a disclosed H30R subclone can comprise H30R1 or H30Rx and can be multi-drug resistant. In an aspect, a disclosed E. coli bacteria can be multi-drug resistant. In an aspect, a disclosed E. coli bacteria can be fluoroquinolone resistant. In an aspect, a disclosed E. coli bacteria can comprise sequence type 131 (ST131) E. coli bacteria and can be fluoroquinolone resistant. In an aspect, a disclosed ST131 E. coli bacteria can comprise a H30R subclone and can be fluoroquinolone resistant. In an aspect, a disclosed H30R subclone can comprise H30R1 or H30Rx and can be fluoroquinolone resistant.

In an aspect, a disclosed method of increasing resistance to a drug-resistant E. coli infection and/or colonization in a subject can further comprise obtaining one or more biological samples from the subject. In an aspect, a disclosed method of reducing the degree and/or severity of a drug-resistant E. coli colonization in a subject can further comprise repeating the obtaining of one or more biological samples from the subject.

In an aspect, the repeating of the obtaining step can occur at different times. In an aspect, the first obtaining step can be performed prior to administering any treatment to the subject. In an aspect, the second obtaining step can be performed after administering any treatment to the subject. In an aspect, subsequent obtaining steps can be performed after administering any treatment to the subject.

In an aspect, a disclosed method of increasing resistance to a drug-resistant E. coli infection and/or colonization in a subject can further comprise comparing the prevalence and/or proportional abundance of one or more bacteria in the subject's gut microbiome to one or more standard levels. In an aspect, a disclosed method of increasing resistance to a drug-resistant E. coli infection and/or colonization in a subject can further comprise comparing the prevalence and/or proportional abundance of one or more bacteria in the subject's gut microbiome to one or more control levels.

In an aspect, a disclosed method of increasing resistance to a drug-resistant E. coli infection and/or colonization in a subject can further comprise comparing the prevalence and/or proportional abundance of the one or more bacteria in the subject's gut microbiome to one or more standard levels, and if the prevalence and/or proportional abundance of the one or more bacteria in the subject's gut microbiome is similar to that of one or more subjects having demonstrated responsiveness to treatment, then characterizing the subject as likely to be responsive to treatment.

In an aspect, a disclosed method of increasing resistance to a drug-resistant E. coli infection and/or colonization in a subject can further comprise comparing the prevalence and/or proportional abundance of the one or more bacteria in the subject's gut microbiome to one or more standard levels, and if the prevalence and/or proportional abundance of the one or more bacteria in the subject's gut microbiome is dissimilar to that of one or more subjects having demonstrated responsiveness to treatment, then characterizing the subject as likely to be non-responsive to treatment.

In an aspect, a disclosed method of increasing resistance to a drug-resistant E. coli infection and/or colonization in a subject can further comprise comparing the prevalence and/or proportional abundance of the one or more bacteria in the subject's gut microbiome to one or more standard levels, and if the prevalence and/or proportional abundance of the one or more bacteria in the subject's gut microbiome is similar to that of one or more subjects having demonstrated non-responsiveness to treatment, then characterizing the subject as likely to be non-responsive to treatment. In an aspect, a disclosed method of increasing resistance to a drug-resistant E. coli infection and/or colonization in a subject can further comprise comparing the prevalence and/or proportional abundance of the one or more bacteria in the subject's gut microbiome to one or more standard levels, and if the prevalence and/or proportional abundance of the one or more bacteria in the subject's gut microbiome is dissimilar to that of one or more subjects having demonstrated non-responsiveness to treatment, then characterizing the subject as likely to be responsive to treatment.

In an aspect, a disclosed method of increasing resistance to a drug-resistant E. coli infection and/or colonization in a subject can further comprise generating a standard level or a standard range for prevalence and/or proportional abundance of one or more bacteria in a gut microbiome. In an aspect, a disclosed method of increasing resistance to a drug-resistant E. coli infection and/or colonization in a subject can further comprise generating a standard level or a standard range for prevalence and/or proportional abundance of one or more bacteria in a gut microbiome, wherein the standard level or the standard range represents a likelihood of non-responsiveness to treatment. In an aspect, a disclosed method of increasing resistance to a drug-resistant E. coli infection and/or colonization in a subject can further comprise generating a standard level or a standard range for prevalence and/or proportional abundance of one or more bacteria in a gut microbiome, wherein the standard level or the standard range represents a likelihood of responsiveness to treatment. In an aspect, a disclosed method of increasing resistance to a drug-resistant E. coli infection and/or colonization in a subject can further comprise treating the subject.

In an aspect, treating the subject can comprise administering a composition comprising (i) one or more agents that can reduce the intestinal carriage associated with the colonization of drug-resistant E. coli bacteria, (ii) one or more agents that can reduce the proportional abundance of one or more bacteria associated with the carriage and/or persistence of drug-resistant E. coli bacteria, (iii) one or more agents that can inhibit proliferation of Asaccharobacter, Bifidobacterium, Collinsella, Eggerthella, Gardnerella, Gordonibacter, or any combination thereof, (iv) one or more agents that can increase proliferation of Alistipes and/or Akkermansia.

In an aspect, a disclosed method of increasing resistance to a drug-resistant E. coli infection and/or colonization in a subject can further comprise comprising administering to the subject a therapeutically effective amount of one or more therapeutic agent. In an aspect, therapeutic agents can comprise any therapeutic agent disclosed herein. In an aspect, treating can comprise administering to the subject a therapeutically effective amount of one or more disclosed compositions. In an aspect, treating can comprise administering to the subject a therapeutically effective amount of one or more disclosed pharmaceutical formulations.

In an aspect, a disclosed sample can comprise a fecal sample. In an aspect, a disclosed fecal sample can be obtained using a fecal swab. In an aspect, the prevalence and/or the proportional abundance of the one or more bacterial in a sample can be generated by sequencing the 16S rRNA gene V3-V4 region. In an aspect, sequencing the 16S rRNA gene V3-V4 region can be performed according to the method described in as described in Fadrosh D W, et al. (2014) Microbiome. 2 (1): 6 by using MiSeq Reagent Kit v3 (600-cycle) (Illumina Inc., San Diego, CA). In an aspect, one or more negative extraction controls (NECs) can be included with each batch of extraction and can be sequenced to assess for cross contamination. In an aspect, one or more no-template controls (NTCs) and one or more positive-template controls (PTCs) can be included to assess for cross-contamination and to verify PCR performance.

In an aspect, a disclosed method of increasing resistance to a drug-resistant E. coli infection and/or colonization in a subject can further comprise repeating the treating step one or more times. In an aspect, a disclosed method of increasing resistance to a drug-resistant E. coli infection and/or colonization in a subject can further comprise repeating the treating step until the subject experiences an alleviation and/or a diminishment of one or more symptoms. In an aspect, the one or more symptoms can be associated with and/or due to the presence of one or more disclosed bacteria in the subject's gut microbiome. In an aspect, the presence of the one or more bacteria in the subject's gut microbiome can interfere with treating the subject for one or more diseases, infections, and/or disorder.

In an aspect, the presence of the one or more ST131 E. coli bacteria in the subject's gut microbiome can interfere with treating the subject for one or more diseases, infections, and/or disorder. In an aspect, the presence of the H30R1 E. coli bacteria and/or H30Rx E. coli bacteria in the subject's gut microbiome can interfere with treating the subject for one or more diseases, infections, and/or disorder. In an aspect, interfering can comprise disrupting and/or diminishing the efficacy of one or more treatments. In an aspect, interfering can comprise rendering the subject resistant to one or more antibiotics. In an aspect, interfering can comprise rendering the subject resistant to fluoroquinolone. In an aspect, the one or more E. coli bacteria can comprise sequence type 131 (ST131) and can be multi-drug resistant. In an aspect, the one or more ST131 E. coli bacteria can comprise a H30R subclone and can be multi-drug resistant. In an aspect, the one or more H30R subclones can comprise H30R1 or H30Rx and is multi-drug resistant. In an aspect, the one or more E. coli bacteria can be multi-drug resistant. In an aspect, the one or more E. coli bacteria can be fluoroquinolone resistant. In an aspect, the one or more E. coli bacteria can comprise sequence type 131 (ST131) E. coli bacteria and can be fluoroquinolone resistant. In an aspect, the one or more ST131 E. coli bacteria can comprise a H30R subclone and can be fluoroquinolone resistant. In an aspect, the one or more H30R subclones can comprise H30R1 or H30Rx and is fluoroquinolone resistant.

In an aspect, the one or more bacteria can comprise bacteria in the Actinobacter phylum, the Bacteroidetes phylum, the Firmicutes phylum, the Verruncomicrobia phylum, the Proteobacteria phylum, or any combination thereof. In an aspect, a higher proportional abundance of Actinobacteria can indicate that the subject is not likely to be responsive to antibiotic treatment. In an aspect, a lower proportional abundance of Actinobacteria can indicate that the subject is likely to be responsive to antibiotic treatment. In an aspect, a higher proportional abundance of Collinsella can indicate that the subject is not likely to be responsive to antibiotic treatment. In an aspect, a lower proportional abundance of Collinsella can indicate that the subject is likely to be responsive to antibiotic treatment. In an aspect, a lower proportional abundance of Verrucomicrobia can indicate that the subject is not likely to be responsive to antibiotic treatment. In an aspect, a higher proportional abundance of Verrucomicrobia can indicate that the subject is likely to be responsive to antibiotic treatment. In an aspect, a higher proportional abundance of Alistipes can indicate that the subject is likely to be responsive to antibiotic treatment. In an aspect, a lower proportional abundance of Alistipes can indicate that the subject is not likely to be responsive to antibiotic treatment. In an aspect, a higher proportional abundance of Akkermansia can indicate that the subject is likely to be responsive to antibiotic treatment. In an aspect, a lower proportional abundance of Akkermansia can indicate that the subject is not likely to be responsive to antibiotic treatment.

In an aspect, a higher proportional abundance of Actinobacteria can indicate a likelihood that the subject will be non-responsive to antibiotic treatment. In an aspect, a lower proportional abundance of Actinobacteria can indicate a likelihood that the subject will be responsive to antibiotic treatment. In an aspect, a higher proportional abundance of Collinsella can indicate a likelihood that the subject will be non-responsive to antibiotic treatment. In an aspect, a lower proportional abundance of Collinsella can indicate a likelihood that the subject will be responsive to antibiotic treatment. In an aspect, a lower proportional abundance of Verrucomicrobia can indicate a likelihood that the subject will be non-responsive to antibiotic treatment. In an aspect, a higher proportional abundance of Verrucomicrobia can indicate a likelihood that the subject will be responsive to antibiotic treatment. In an aspect, a higher proportional abundance of Alistipes can indicate a likelihood that the subject will be responsive to antibiotic treatment. In an aspect, a lower proportional abundance of Alistipes can indicate a likelihood that the subject will be non-responsive to antibiotic treatment. In an aspect, a higher proportional abundance of Akkermansia can indicate a likelihood that the subject will be responsive to antibiotic treatment. In an aspect, a lower proportional abundance of Akkermansia can indicate a likelihood that the subject will be non-responsive to antibiotic treatment.

In an aspect of a disclosed method of increasing resistance to a drug-resistant E. coli infection and/or colonization in a subject, a subject is likely to be responsive to antibiotic treatment when there is (i) a lower proportional abundance of Actinobacteria; (ii) a lower proportional abundance of Collinsella; (iii) a higher proportional abundance of Verrucomicrobia; (iv) a higher proportional abundance of Alistipes; (v) a higher proportional abundance of Akkermansia; or (vi) any combination thereof. In an aspect of a disclosed method of increasing resistance to a drug-resistant E. coli infection and/or colonization in a subject, a subject is not likely to be responsive to antibiotic treatment when there is (i) a higher proportional abundance of Actinobacteria; (ii) a higher proportional abundance of Collinsella; (iii) a lower proportional abundance of Verrucomicrobia; (iv) a lower proportional abundance of Alistipes; (v) a lower proportional abundance of Akkermansia; or (vi) any combination thereof.

In an aspect, a disclosed method of increasing resistance to a drug-resistant E. coli infection and/or colonization in a subject can further comprise further comprising measuring the prevalence and/or proportional abundance of one or more E. coli bacteria sequence type 131 (ST131). In an aspect, the one or more ST131 E. coli bacteria can comprise a H30R subclone and is fluoroquinolone resistant. In an aspect, the H30R subclone can comprise H30R1 or H30Rx and can be fluoroquinolone resistant.

In an aspect, a disclosed method of increasing resistance to a drug-resistant E. coli infection and/or colonization in a subject can further comprise administering to the subject one or more compositions that modulate the intestinal carriage and persistence of one or more ST131 E. coli bacteria. In an aspect, a disclosed method of increasing resistance to a drug-resistant E. coli infection and/or colonization in a subject can further comprise administering to the subject one or more compositions that modulate the intestinal carriage and persistence of H30R1 and/or H30Rx. In an aspect, a disclosed method of increasing resistance to a drug-resistant E. coli infection and/or colonization in a subject can further comprise administering to the subject one or more compositions that decrease and/or disrupt the intestinal carriage and persistence of one or more ST131 E. coli bacteria. In an aspect, a disclosed method of increasing resistance to a drug-resistant E. coli infection and/or colonization in a subject can further comprise administering to the subject one or more compositions that decrease and/or disrupt the intestinal carriage and persistence of H30R1 and/or H30Rx. In an aspect, a disclosed method of increasing resistance to a drug-resistant E. coli infection and/or colonization in a subject can further comprise administering to the subject one or more compositions that inhibit proliferation of H30R1 and/or H30Rx in the subject's gut microbiome and/or gastrointestinal tract.

In an aspect, a disclosed method of increasing resistance to a drug-resistant E. coli infection and/or colonization in a subject can further comprise administering to the subject one or more compositions that modulate the intestinal carriage and persistence of one or more bacteria. In an aspect, a disclosed method of increasing resistance to a drug-resistant E. coli infection and/or colonization in a subject can further comprise administering to the subject one or more compositions that decrease and/or disrupt the intestinal carriage and persistence of one or more bacteria. In an aspect, a disclosed method of increasing resistance to a drug-resistant E. coli infection and/or colonization in a subject can further comprise administering to the subject one or more compositions that inhibit proliferation of one or more bacteria in the subject's gut microbiome and/or gastrointestinal tract In an aspect, a disclosed method of increasing resistance to a drug-resistant E. coli infection and/or colonization in a subject can further comprise administering to the subject one or more compositions that inhibit proliferation of one or more bacteria belonging to the Actinobacteria phylum in the subject's gut and/or gastrointestinal tract. In an aspect, a disclosed method of increasing resistance to a drug-resistant E. coli infection and/or colonization in a subject can further comprise administering to the subject one or more compositions that inhibit proliferation of Asaccharobacter, Bifidobacterium, Collinsella, Eggerthella, Gardnerella, Gordonibacter, or any combination thereof in the subject's gut and/or gastrointestinal tract.

In an aspect, a disclosed method of increasing resistance to a drug-resistant E. coli infection and/or colonization in a subject can further comprise administering to the subject one or more compositions that inhibit proliferation of Collinsella in the subject's gut and/or gastrointestinal tract. In an aspect, a disclosed method of increasing resistance to a drug-resistant E. coli infection and/or colonization in a subject can further comprise administering to the subject one or more compositions that inhibit proliferation of Gardnerella and/or Gordonibacter in the subject's gut and/or gastrointestinal tract. In an aspect, a disclosed method of increasing resistance to a drug-resistant E. coli infection and/or colonization in a subject can further comprise administering to the subject one or more compositions that inhibit proliferation of one or more bacteria belonging to the Firmicutes phylum in the subject's gut and/or gastrointestinal tract. In an aspect, a disclosed method of increasing resistance to a drug-resistant E. coli infection and/or colonization in a subject can further comprise administering to the subject one or more compositions that inhibit proliferation of Enterococcus, Lactobacillus, Streptococcus, Weissella, or any combination thereof in the subject's gut and/or gastrointestinal tract. In an aspect, a disclosed method of increasing resistance to a drug-resistant E. coli infection and/or colonization in a subject can further comprise administering to the subject one or more compositions that inhibit proliferation of Lactobacillus and/or Streptococcus in the subject's gut and/or gastrointestinal tract.

In an aspect, a disclosed method of increasing resistance to a drug-resistant E. coli infection and/or colonization in a subject can further comprise administering to the subject one or more compositions that decrease and/or disrupt the intestinal carriage and persistence of one or more bacteria. In an aspect, a disclosed method of increasing resistance to a drug-resistant E. coli infection and/or colonization in a subject can further comprise administering to the subject one or more compositions that increase and/or stimulate proliferation of one or more bacteria in the subject's gut microbiome and/or gastrointestinal tract. In an aspect, a disclosed method of increasing resistance to a drug-resistant E. coli infection and/or colonization in a subject can further comprise administering to the subject one or more compositions that increase and/or stimulate proliferation of one or more bacteria belonging to the Bacteroidetes phylum in the subject's gut microbiome and/or gastrointestinal tract. In an aspect, a disclosed method of increasing resistance to a drug-resistant E. coli infection and/or colonization in a subject can further comprise administering to the subject one or more compositions that increase and/or stimulate proliferation of one or more bacteria belonging to the Verrucomicrobia phylum in the subject's gut microbiome and/or gastrointestinal tract. In an aspect, a disclosed method of increasing resistance to a drug-resistant E. coli infection and/or colonization in a subject can further comprise administering to the subject one or more compositions that increase and/or stimulate proliferation of Alistipes and/or Akkermansia the subject's gut microbiome and/or gastrointestinal tract. In an aspect, a lower proportional abundance of Verrucomicrobia can indicate that the subject is not likely to be responsive to antibiotic treatment. In an aspect, a higher proportional abundance of Verrucomicrobia can indicate that the subject is likely to be responsive to antibiotic treatment. In an aspect, a higher proportional abundance of Alistipes can indicate that the subject is likely to be responsive to antibiotic treatment. In an aspect, a lower proportional abundance of Alistipes can inhibit that the subject is not likely to be responsive to antibiotic treatment. In an aspect, a higher proportional abundance of Akkermansia can inhibit that the subject is likely to be responsive to antibiotic treatment. In an aspect, a lower proportional abundance of Akkermansia can inhibit that the subject is not likely to be responsive to antibiotic treatment.

In an aspect of a disclosed method of increasing resistance to a drug-resistant E. coli infection and/or colonization in a subject, a subject at risk can be any subject. In an aspect, an at-risk subject can comprise an adult, a young adult, a child, a toddler, or an infant. In an aspect, an at-risk subject can have cancer. In an aspect, an at-risk subject can be receiving chemotherapy. In an aspect, an at-risk subject can be receiving immunosuppressive therapy. In an aspect, an at-risk subject that does not have an infection and/or is not suspected of having an infection. In an aspect, following administration, the colonization of a pathogenic E. coli in the subject's intestinal tract and/or gut can be inhibited and/or prevented. In an aspect, a disclosed method of increasing resistance to a drug-resistant E. coli infection and/or colonization in a subject can further comprise promoting respiratory health in a subject. In an aspect, a disclosed biotherapeutic can inhibit and/or prevent the growth of one or more E. coli in one or more parts of the subject's respiratory system. In an aspect, a disclosed method of increasing resistance to a drug-resistant E. coli infection and/or colonization in a subject can further comprise characterizing the microbiome of a biological sample. In an aspect, a disclosed microbiome can comprise the gut microbiome, the gastrointestinal microbiome, or both. In an aspect, a disclosed method of increasing resistance to a drug-resistant E. coli infection and/or colonization in a subject can further comprise obtaining a biological sample from the subject. In an aspect, a disclosed biological sample can comprise a stool sample, a fecal swab, or any combination thereof.

In an aspect of a disclosed method of increasing resistance to a drug-resistant E. coli infection and/or colonization in a subject, characterizing the microbiome can comprise (i) collecting a biological sample from the subject; (ii) extracting nucleic acid from the subject's biological sample; and (iii) sequencing the extracted nucleic acid. In an aspect, sequencing the extracted nucleic acid can generate sequence data. In an aspect, characterizing the microbiome can further comprise analyzing the sequence data using taxonomic classification. In an aspect, using taxonomic classification can comprise PCR amplification. In an aspect, PCR amplification can comprise using primers targeting the 16S rRNA gene. In an aspect, PCR amplification can comprise primers targeting the V4 variable region of the 16S rRNA gene. In an aspect, disclosed primers can comprise a pair of primers.

In an aspect, a disclosed method of increasing resistance to a drug-resistant E. coli infection and/or colonization in a subject can further comprise identifying the one or more pathogenic bacteria. In an aspect, a disclosed method of increasing resistance to a drug-resistant E. coli infection and/or colonization in a subject can further comprise diagnosing the subject with an infection.

In an aspect of a disclosed method of increasing resistance to a drug-resistant E. coli infection and/or colonization in a subject, a disclosed therapeutically effective dose can comprise a total of at least 100 bacterial CFUs of the one or more probiotics, a total of at least 200 to at least 300 bacterial CFUs of the one or more probiotics, a total of at least 300 to at least 400 bacterial CFUs of the one or more probiotics, a total of at least 400 to at least 500 bacterial CFUs of the one or more probiotics, a total of at least 500 to at least 600 bacterial CFUs of the one or more probiotics, a total of at least 600 to at least 700 bacterial CFUs of the one or more probiotics, a total of at least 700 to at least 800 bacterial CFUs of the one or more probiotics, a total of at least 800 to at least 900 bacterial CFUs of the one or more probiotics, a total of at least 900 to at least 1000 bacterial CFUs of the one or more probiotics, or a total of at least 1000 bacterial CFUs of one or more disclosed probiotics (such as, for example, at least one strain from the bacterial genus Alistipes and/or at least one strain from the bacterial genus Akkermansia).

In an aspect of a disclosed pharmaceutical formulation, a disclosed therapeutically effective dose can comprise a total of at least 1000 to at least 2000 bacterial CFUs of the one or more probiotics, a total of at least 2000 to at least 3000 bacterial CFUs of the one or more probiotics, a total of at least 3000 to at least 4000 bacterial CFUs of the one or more probiotics, a total of at least 4000 to at least 5000 bacterial CFUs of the one or more probiotics, a total of at least 5000 to at least 6000 bacterial CFUs of the one or more probiotics, a total of at least 6000 to at least 7000 bacterial CFUs of the one or more probiotics, a total of at least 8000 to at least 9000 bacterial CFUs of the one or more probiotics, or a total of at least 10,000 bacterial CFUs of one or more disclosed probiotics (such as, for example, at least one strain from the bacterial genus Alistipes and/or at least one strain from the bacterial genus Akkermansia).

In an aspect of a disclosed method of increasing resistance to a drug-resistant E. coli infection and/or colonization in a subject, a disclosed therapeutically effective dose can comprise a total of at least 100 bacterial CFUs of at least one strain from the bacterial genus Alistipes and/or at least one strain from the bacterial genus Akkermansia, a total of at least 200 to at least 300 bacterial CFUs of at least one strain from the bacterial genus Alistipes and/or at least one strain from the bacterial genus Akkermansia, a total of at least 300 to at least 400 bacterial CFUs of at least one strain from the bacterial genus Alistipes and/or at least one strain from the bacterial genus Akkermansia, a total of at least 400 to at least 500 bacterial CFUs of at least one strain from the bacterial genus Alistipes and/or at least one strain from the bacterial genus Akkermansia, a total of at least 500 to at least 600 bacterial CFUs of at least one strain from the bacterial genus Alistipes and/or at least one strain from the bacterial genus Akkermansia, a total of at least 600 to at least 700 bacterial CFUs of at least one strain from the bacterial genus Alistipes and/or at least one strain from the bacterial genus Akkermansia, a total of at least 700 to at least 800 bacterial CFUs of at least one strain from the bacterial genus Alistipes and/or at least one strain from the bacterial genus Akkermansia, a total of at least 800 to at least 900 bacterial CFUs of at least one strain from the bacterial genus Alistipes and/or at least one strain from the bacterial genus Akkermansia, a total of at least 900 to at least 1000 bacterial CFUs of at least one strain from the bacterial genus Alistipes and/or at least one strain from the bacterial genus Akkermansia, or a total of at least 1000 bacterial CFUs of at least one strain from the bacterial genus Alistipes and/or at least one strain from the bacterial genus Akkermansia.

In an aspect of a disclosed method of increasing resistance to a drug-resistant E. coli infection and/or colonization in a subject, a disclosed therapeutically effective dose can comprise a total of at least 1000 to at least 2000 bacterial CFUs of at least one strain from the bacterial genus Alistipes and/or at least one strain from the bacterial genus Akkermansia, a total of at least 2000 to at least 3000 bacterial CFUs of at least one strain from the bacterial genus Alistipes and/or at least one strain from the bacterial genus Akkermansia, a total of at least 3000 to at least 4000 bacterial CFUs of at least one strain from the bacterial genus Alistipes and/or at least one strain from the bacterial genus Akkermansia, a total of at least 4000 to at least 5000 bacterial CFUs of at least one strain from the bacterial genus Alistipes and/or at least one strain from the bacterial genus Akkermansia, a total of at least 5000 to at least 6000 bacterial CFUs of at least one strain from the bacterial genus Alistipes and/or at least one strain from the bacterial genus Akkermansia, a total of at least 6000 to at least 7000 bacterial CFUs of at least one strain from the bacterial genus Alistipes and/or at least one strain from the bacterial genus Akkermansia, a total of at least 8000 to at least 9000 bacterial CFUs of at least one strain from the bacterial genus Alistipes and/or at least one strain from the bacterial genus Akkermansia, or a total of at least 10,000 bacterial CFUs of at least one strain from the bacterial genus Alistipes and/or at least one strain from the bacterial genus Akkermansia.

In an aspect of a disclosed method of increasing resistance to a drug-resistant E. coli infection and/or colonization in a subject, a disclosed therapeutically effective dose can comprise a total of at least 10⁴ to at least 10⁵ bacterial CFUs of the one or more probiotics, a total of at least 10⁵ to at least 10⁶ bacterial CFUs of the one or more probiotics, a total of at least 10⁶ to at least 10⁷ bacterial CFUs of the one or more probiotics, a total of at least 10⁷ to at least 10⁸ bacterial CFUs of the one or more probiotics, a total of at least 10⁸ to at least 10⁹ bacterial CFUs of the one or more probiotics, a total of at least 10⁹ to at least 10¹⁰ bacterial CFUs of the one or more probiotics, a total of at least 10¹⁰ to at least 10¹¹ bacterial CFUs of the one or more probiotics, a total of at least 10¹¹ to at least 10¹² bacterial CFUs of the one or more probiotics, a total of at least 10¹² bacterial CFUs of the one or more probiotics, or more than 10¹² bacterial CFUs of one or more disclosed probiotics (such as, for example, at least one strain from the bacterial genus Alistipes and/or at least one strain from the bacterial genus Akkermansia).

In an aspect of a disclosed method of increasing resistance to a drug-resistant E. coli infection and/or colonization in a subject, a disclosed therapeutically effective dose can comprise at least 10³, 10⁴, 10⁵, 10⁶, 10⁷, 10⁸, 10⁹, 10¹⁰, 10¹¹, or 10¹² CFUs of a disclosed probiotic or a consortium of disclosed probiotics (such as, for example, at least one strain from the bacterial genus Alistipes and/or at least one strain from the bacterial genus Akkermansia), preferably at least 1.2×10³, 1.4×10³, 2×10³, or 3×10³ CFUs of a disclosed probiotic or a consortium of disclosed probiotics, at least 5×10³, 5.02×10³, 5.04×10³, 5.2×10³, or 5.4×10³ CFUs of a disclosed probiotic or a consortium of disclosed probiotics, at least 1.02×10⁴, 1.04×10⁴, 1.2×10⁴, 1.4×10⁴, 1.5×10⁴, 2×10⁴, or 3×10⁴ CFUs of a disclosed probiotic or a consortium of disclosed probiotics, at least 5×10⁴, 5.02×10⁴, 5.04×10⁴, 5.2×10⁴, or 5.4×10⁴ CFUs of a disclosed probiotic or a consortium of disclosed probiotics, at least 1.02×10⁵, 1.04×10⁵, 1.2×10⁵, 1.4×10⁵, 1.5×10⁵, 2×10⁵, or 3×10⁵ CFUs of a disclosed probiotic or a consortium of disclosed probiotics, at least 1.02×10⁶, 1.04×10⁶, 1.2×10⁶, 1.4×10⁶, 2×10⁶, or 3×10⁶ CFUs of a disclosed probiotic or a consortium of disclosed probiotics, at least 1.02×10⁷, 1.04×10⁷, 1.2×10⁷, 1.4×10⁷, 2×10⁷, or 3×10⁷ CFUs of a disclosed probiotic or a consortium of disclosed probiotics, at least 1.02×10⁸, 1.04×10⁸, 1.2×10⁸, 1.4×10⁸, 2×10⁸, or 3×10⁸ CFUs of a disclosed probiotic or a consortium of disclosed probiotics, at least 1.02×10⁹, 1.04×10⁹, 1.2×10⁹, 1.4×10⁹, 2×10⁹, or 3×10⁹ CFUs of a disclosed probiotic or a consortium of disclosed probiotics, at least 10¹⁰ CFUs of a disclosed probiotic or a consortium of disclosed probiotics, at least 1.02×10¹⁰, 1.04×10¹⁰, 1.2×10¹⁰, 1.4×10¹⁰, 2×10¹⁰, or 3×10¹⁰ CFUs of a disclosed probiotic or a consortium of disclosed probiotics, at least 1.02×10¹¹, 1.04×10¹¹, 1.2×10¹¹, 1.4×10¹¹, 2×10¹¹, or 3×10¹¹ CFUs of a disclosed probiotic or a consortium of disclosed probiotics, or at least 1.02×10¹², 1.04×10¹², 1.2×10¹², 1.4×10¹², 2×10¹², or 3×10¹² CFUs of a disclosed probiotic or a consortium of disclosed probiotics per dose.

In an aspect, a disclosed therapeutically effective dose can comprise any amount or an unlimited amount of bacterial CFUs of the one or more probiotics.

In an aspect, a disclosed method of increasing resistance to a drug-resistant E. coli infection and/or colonization in a subject can further comprise treating the subject. In an aspect, treating the subject can comprise treating the subject's respiratory infection. In an aspect, treating the subject can comprise treating the subject's non-respiratory infection.

In an aspect, a disclosed method of increasing resistance to a drug-resistant E. coli infection and/or colonization in a subject can further comprise administering to the subject a therapeutically effective amount of one or more anti-bacterial agents. Anti-bacterial agents and combinations of anti-bacterial agents are known to the art and discussed supr.

In an aspect, a disclosed method of increasing resistance to a drug-resistant E. coli infection and/or colonization in a subject can further comprise administering to the subject a therapeutically effective amount of (i) one or more active agents, (ii) one or more biologically active agents, (iii) one or more pharmaceutically active agents, (iv) one or more immune-based therapeutic agents, (v) one or more clinically approved agents, or (vi) any combination thereof. In an aspect, a therapeutically effective amount of an active agent, a biologically active agent, a pharmaceutically active agent, an immune-based therapeutic active agent, a clinically approved agent, or an anti-bacterial agent can be about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, a disclosed method of increasing resistance to a drug-resistant E. coli infection and/or colonization in a subject can further comprise administering to the subject one or more therapeutic agents. In an aspect, a disclosed therapeutic agent can comprise a biologically active agent, a pharmaceutically active agent, an anti-bacterial agent, an anti-fungal agent, an anti-viral agent, a corticosteroid, an analgesic, an immunostimulant, an immune-based product, or any combination thereof. In an aspect of a disclosed method of increasing resistance to a drug-resistant E. coli infection and/or colonization in a subject, administering can comprise intranasal administration, oral administration, sublingual administration, or any combination thereof.

In an aspect, a disclosed method of increasing resistance to a drug-resistant E. coli infection and/or colonization in a subject, a subject can be a healthy subject, for example, a subject that does not have an infection and/or is not suspected of having an infection.

In an aspect, a subject can be an adult, a child, or an infant. In an aspect, a subject can be a neonate. In an aspect, a subject can be a premature infant. In an aspect, a subject can be immune-compromised. In an aspect, a subject can have diabetes or a chronic disease (e.g., heart disease, kidney disease, or liver disease). In an aspect, a subject can have HIV. In an aspect, a subject can have cancer or has had cancer. In an aspect, a subject can be the recipient of one or more solid organ transplants. In an aspect, a female subject can be pregnant. In an aspect, a subject can have or can be suspected of having a urinary tract infection (UTI). In an aspect, a subject can be in a high-risk environment (e.g., an acute care facility, a hospital, a longer-term acute care hospital, an in-patient facility, an in-patient rehabilitation facility, or a battle-zone or active war zone). In an aspect, a subject can be in the midst of a humanitarian crisis.

In an aspect, a subject can be diagnosed with an infection of one or more parts of the respiratory system. In an aspect, a disclosed therapeutically effective amount of a biotherapeutic can comprise an amount sufficient to inhibit and/or prevent the growth of one or more pathogenic bacteria in one or more parts of the subject's gastrointestinal system.

In an aspect, a disclosed method of increasing resistance to a drug-resistant E. coli infection and/or colonization in a subject can comprise a 10%, 20%, 30%, 40%, 50%, 60%, 70%, 80%, 90%, 100%, or any amount of inhibiting and/or preventing the growth and/or spread of one or more pathogenic bacteria (e.g., ST131 (H30R1 and/or H30Rx)) when compared to a control subject (such as, for example, a subject that has not received a disclosed biotherapeutic). In an aspect, a disclosed method of increasing resistance to a drug-resistant E. coli infection and/or colonization in a subject can comprise a 10-20%, 20-30%, 30-40%, 40-50%, 50-60%, 60-70%, 70-80%, 80-90%, or 90-100% or any amount of inhibiting and/or preventing the growth and/or spread of one or more pathogenic bacteria (e.g., ST131 (H30R1 and/or H30Rx)) when compared to a control subject (such as, for example, a subject that has not received a disclosed biotherapeutic).

In an aspect, a disclosed method of increasing resistance to a drug-resistant E. coli infection and/or colonization in a subject can comprise a 10%, 20%, 30%, 40%, 50%, 60%, 70%, 80%, 90%, 100%, or any amount of inhibiting and/or preventing the adherence and/or colonization of one or more pathogenic bacteria (e.g., ST131 (H30R1 and/or H30Rx)) when compared to a control subject (such as, for example, a subject that has not received a disclosed biotherapeutic). In an aspect, a disclosed method of increasing resistance to a drug-resistant E. coli infection and/or colonization in a subject can comprise a 10-20%, 20-30%, 30-40%, 40-50%, 50-60%, 60-70%, 70-80%, 80-90%, or 90-100% or any amount of inhibiting and/or preventing the adherence and/or colonization of one or more pathogenic bacteria (e.g., ST131 (H30R1 and/or H30Rx)) when compared to a control subject (such as, for example, a subject that has not received a disclosed biotherapeutic).

In an aspect, a disclosed method of increasing resistance to a drug-resistant E. coli infection and/or colonization in a subject can comprise a 10%, 20%, 30%, 40%, 50%, 60%, 70%, 80%, 90%, 100%, or any amount of modulating the endocytosis or paracellular migration of one or more pathogenic bacteria (e.g., ST131 E. coli (i.e., H30R1 and/or H30Rx)) when compared to a control subject (such as, for example, a subject that has not received a disclosed biotherapeutic). In an aspect, a disclosed method of increasing resistance to a drug-resistant E. coli infection and/or colonization in a subject can comprise a 10-20%, 20-30%, 30-40%, 40-50%, 50-60%, 60-70%, 70-80%, 80-90%, or 90-100% or any amount of modulating the endocytosis or paracellular migration of one or more pathogenic bacteria (e.g., ST131 E. coli (i.e., H30R1 and/or H30Rx)) when compared to a control subject (such as, for example, a subject that has not received a disclosed biotherapeutic).

In an aspect, a disclosed method of increasing resistance to a drug-resistant E. coli infection and/or colonization in a subject can comprise a 10%, 20%, 30%, 40%, 50%, 60%, 70%, 80%, 90%, 100%, or any amount of inhibiting and/or preventing the spread of one or more pathogenic bacteria (e.g., ST131 (H30R1 and/or H30Rx) by competitive adherence and/or niche occupancy when compared to a control subject (such as, for example, a subject that has not received a disclosed biotherapeutic). In an aspect, a disclosed method of increasing resistance to a drug-resistant E. coli infection and/or colonization in a subject can comprise a 10-20%, 20-30%, 30-40%, 40-50%, 50-60%, 60-70%, 70-80%, 80-90%, or 90-100% or any amount of inhibiting and/or preventing the spread of one or more pathogenic bacteria (e.g., ST131 (H30R1 and/or H30Rx)) by competitive adherence and/or niche occupancy when compared to a control subject (such as, for example, a subject that has not received a disclosed biotherapeutic).

In an aspect, a disclosed method of increasing resistance to a drug-resistant E. coli infection and/or colonization in a subject can comprise repeating the administering of one or more disclosed biotherapeutics, one or more disclosed secreted factors, a disclosed consortium of probiotics, one or more disclosed probiotics, or any combination thereof. For example, in an aspect, repeating the administering can comprise administering one or more times daily (e.g., 1, 2, 3, or 4 times). In an aspect, repeating the administering can comprise administering on several consecutive days (e.g., for 1, 2, 3, 4, 5, 6, 7, 8, 9, or 10 days, or more than 10 days), weeks (e.g., for 1, 2, 3, 4, 5, 6, 7, 8, 9, or 10 weeks, or more than 10 weeks), or months (e.g., for 1, 2, 3, 4, 5, or 6 months, or more than 6 months). In an aspect, a disclosed biotherapeutic, a disclosed secreted factor, a disclosed consortium of probiotics, a disclosed probiotic, or any combination thereof can be administered 1-3 times per day for about 7 to 21 days.

In an aspect, a disclosed method of increasing resistance to a drug-resistant E. coli infection and/or colonization in a subject in a subject can comprise monitoring the subject. In an aspect, monitoring the subject can comprise monitoring the subject for the development of adverse effects. In an aspect, in the absence of adverse effects, a disclosed method of increasing resistance to a drug-resistant E. coli infection and/or colonization in a subject can comprise continuing to treat the subject. In an aspect, continuing to treat the subject can comprise continuing to administer to the subject one or more disclosed biotherapeutics, one or more disclosed secreted factors, a disclosed consortium of probiotics, one or more disclosed probiotics, or any combination thereof. In an aspect, in the presence of adverse effects, a disclosed method of reducing the risk of developing a pathogenic E. coli infection can comprise modifying one or more steps of the method. In an aspect, modifying one or more steps of a disclosed method can comprise modifying the administering step. In an aspect, modifying the administering step can comprise changing the amount of one or more disclosed biotherapeutics, one or more disclosed secreted factors, a disclosed consortium of probiotics, one or more disclosed probiotics, or any combination thereof administered to the subject, changing the frequency of administration of one or more disclosed biotherapeutics, one or more disclosed secreted factors, a disclosed consortium of probiotics, one or more disclosed probiotics, or any combination thereof, changing the duration of administration of one or more disclosed biotherapeutics, one or more disclosed secreted factors, a disclosed consortium of probiotics, one or more disclosed probiotics, or any combination thereof, changing the route of administration of one or more disclosed biotherapeutics, one or more disclosed secreted factors, a disclosed consortium of probiotics, one or more disclosed probiotics, or any combination thereof, or any combination of disclosed changes.

In an aspect, a disclosed method of increasing resistance to a drug-resistant E. coli infection and/or colonization in a subject can comprise manipulating the microbiome to inhibit and/or prevent infection caused by one or more pathogenic bacteria, such as an infection caused by ST131 E. coli (i.e., H30R1 and/or H30Rx). In an aspect, a disclosed method of increasing resistance to a drug-resistant E. coli infection and/or colonization in a subject can comprise manipulating the microbiome to decrease and/or minimize the risk of infection caused by one or more pathogenic bacteria, such as an infection caused by ST131 E. coli (i.e., H30R1 and/or H30Rx).

In an aspect, a disclosed method of increasing resistance to a drug-resistant E. coli infection and/or colonization in a subject can comprise subjecting the subject to one or more invasive or non-invasive diagnostic assessments. Diagnostic assessments are known to the art. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination thereof. In an aspect, a disclosed invasive diagnostic assessment can comprise a tissue biopsy or exploratory surgery.

In an aspect, a disclosed method of increasing resistance to a drug-resistant E. coli infection and/or colonization in a subject, techniques to monitor, measure, and/or assess the restoring one or more aspects of respiratory health can comprise qualitative (or subjective) means as well as quantitative (or objective) means. These means are known to the skilled person. For example, representative regulated variables and sensors relating to systemic homeostasis are discussed supra.

In an aspect, a disclosed method of increasing resistance to a drug-resistant E. coli infection and/or colonization in a subject, a disclosed composition, a disclosed pharmaceutical formulation, a disclosed biotherapeutic, a disclosed bacteria belonging to Alistipes genus and/or a disclosed bacteria belonging to Akkermansia, a disclosed secreted factor, a disclosed consortium of probiotics, a disclosed probiotic (e.g., Alistipes and/or Akkermansia), or any combination thereof can be administered in the absence of identifying the one or more pathogenic bacteria and/or in the absence of characterizing the microbiome. In an aspect of a disclosed method, a disclosed composition, a disclosed pharmaceutical formulation, a disclosed biotherapeutic, a disclosed bacteria belonging to Alistipes genus and/or a disclosed bacteria belonging to Akkermansia, a disclosed secreted factor, a disclosed consortium of probiotics, a disclosed probiotic (e.g., Alistipes and/or Akkermansia), or any combination thereof can be administered on demand.

In an aspect of a disclosed method of increasing resistance to a drug-resistant E. coli infection and/or colonization in a subject, a disclosed biotherapeutic can be formulated for administration as a pharmaceutical formulation. In an aspect, a disclosed pharmaceutical formulation comprising a probiotic, a consortium of probiotics, factors secreted from a probiotic, factors secreted from a consortium of probiotics, or any combination thereof, and a pharmaceutically acceptable carrier and/or excipient can be used in any disclosed method of increasing resistance to a drug-resistant E. coli infection and/or colonization in a subject. Any pharmaceutical formulation disclosed herein can be used in a disclosed method of reducing the risk of developing a pathogenic ST131 E. coli infection (i.e., H30R1 and/or H30Rx).

In an aspect, a disclosed method can further (i) reduce the risk of developing a colonization of drug-resistant E. coli, (ii) reduce the degree and/or severity of a drug-resistant E. coli infection, (iii) reduce the degree and/or severity of a drug-resistant E. coli colonization, (iv) reduce the degree and/or severity of a drug-resistant E. coli colonization, or (v) any combination thereof.

In an aspect, a disclosed method can further comprise administering to the subject one or more broad-spectrum antibiotic or antibiotic agents.

### F. Kits

Disclosed herein is a kit comprising a disclosed composition or a disclosed pharmaceutical formulation. Disclosed herein is a kit comprising a disclosed composition comprising one or more bacteria belonging to the Alistipes genus. Disclosed herein is a kit comprising a disclosed composition comprising one or more bacteria belonging to the Akkermansia genus. Disclosed herein is a kit comprising a disclosed composition comprising one or more bacteria belonging to the Alistipes genus and one or more bacteria belonging to the Akkermansia genus. Disclosed herein is a kit comprising a disclosed composition comprising one or more bacteria belonging to the Alistipes genus and one or more probiotics. Disclosed herein is a kit comprising a disclosed composition comprising one or more bacteria belonging to the Akkermansia genus and one or more probiotics. Disclosed herein is a kit comprising a disclosed composition comprising one or more bacteria belonging to the Alistipes genus and one or more bacteria belonging to the Akkermansia genus and one or more probiotics. Disclosed herein is a kit comprising a disclosed composition comprising one or more bacteria belonging to the Alistipes genus and one or more prebiotics. Disclosed herein is a kit comprising a disclosed composition comprising one or more bacteria belonging to the Akkermansia genus and one or more prebiotics. Disclosed herein is a kit comprising a disclosed composition comprising one or more bacteria belonging to the Alistipes genus and one or more bacteria belonging to the Akkermansia genus and one or more prebiotics. Disclosed herein is a kit comprising a disclosed composition comprising one or more agents that can reduce the intestinal carriage associated with the colonization of drug-resistant E. coli bacteria, thereby reducing the risk of an infection. Disclosed herein is a kit comprising a disclosed composition comprising one or more agents that can reduce the proportional abundance of one or more bacteria associated with the carriage and/or persistence of drug-resistant E. coli bacteria, thereby reducing the degree and/or severity of colonization by a drug-resistance E. coli. Disclosed herein is a kit comprising a disclosed composition comprising one or more agents that can inhibit proliferation of Asaccharobacter, Bifidobacterium, Collinsella, Eggerthella, Gardnerella, Gordonibacter, or any combination thereof in the gut microbiome of the subject. Disclosed herein is a kit comprising a disclosed composition comprising one or more agents that can increase proliferation of Alistipes and/or Akkermansia.

In an aspect, a disclosed kit can further comprise one or more anti-bacterial agents, one or more therapeutic agents, or any combination thereof. “Anti-bacterial agents” and “Therapeutic Agents” are known to the art and are described supra. For example, anti-bacterial agents and combinations of anti-bacterial agents are known to the art and discussed supra.

In an aspect, a disclosed kit can further comprise one or more bacteriophages capable of killing E. coli. In an aspect, a disclosed E. coli-killing bacteriophage can comprise BP539, BP700, BP753, BP814, BP953, BP954, BP970, BP1002, BP1151, BP1155, BP1168, BP1176, BP1197, BP1226, BP1229, or any combination thereof. In an aspect, a disclosed E. coli-killing bacteriophage can comprise MV36.2, JJ2050.2, C3, C19T, JJ6.1, or any combination thereof.

In an aspect, the one or more disclosed biotherapeutics, disclosed secreted factors, disclosed consortiums of probiotics, disclosed probiotics, or any combination thereof can promote respiratory health in a subject, can reduce the risk of developing an infection caused by pathogenic bacteria, can reduce the risk of developing a ST131 E. coli (i.e., H30R1 and/or H30Rx) infection in a subject, can treat and/or prevent ST131 E. coli (i.e., H30R1 and/or H30Rx) colonization and/or infection, can modulate microbial diversity and/or composition, or any combination thereof. In an aspect, the one or more disclosed therapeutic agents in a disclosed kit can treat, prevent, inhibit, and/or ameliorate one or more comorbidities in a subject.

In an aspect, a disclosed kit can comprise at least two components constituting the kit. Together, the components constitute a functional unit for a given purpose (such as, for example, treating a subject diagnosed with or suspected of having an infection such as a respiratory infection). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise (i) one or more of a disclosed biotherapeutic, a disclosed secreted factor, a disclosed consortium of probiotics, a disclosed probiotic, a disclosed pharmaceutical formulation, or any combination thereof, and (ii) a label or package insert with instructions for use. In an aspect, suitable containers include, for example, bottles, vials, syringes, blister pack, etc. The containers can be formed from a variety of materials such as glass or plastic. The container can hold one or more of a disclosed biotherapeutic, a disclosed secreted factor, a disclosed consortium of probiotics, a disclosed probiotic, or any combination, a disclosed pharmaceutical formulation, or any combination thereof, and can have a sterile access port (for example the container may be an intravenous solution bag or a vial having a stopper pierceable by a hypodermic injection needle). The label or package insert can indicate one or more of a disclosed biotherapeutic, a disclosed secreted factor, a disclosed consortium of probiotics, a disclosed probiotic, or any combination, a disclosed pharmaceutical formulation, or any combination thereof can be used for promoting respiratory health in a subject, for reducing the risk of developing an infection caused by pathogenic bacteria, for reducing the risk of developing a ST131 E. coli (i.e., H30R1 and/or H30Rx) infection in a subject, for treating and/or preventing ST131 E. coli (i.e., H30R1 and/or H30Rx) colonization and/or infection, for modulating microbial diversity and/or composition, or any combination thereof. A kit can comprise additional components necessary for administration such as, for example, other buffers, diluents, filters, needles, and syringes. In an aspect, a disclosed kit can be used to promote respiratory health in a subject, to reduce the risk of developing an infection caused by pathogenic bacteria, to reduce the risk of developing a ST131 E. coli (i.e., H30R1 and/or H30Rx) infection in a subject, to treat and/or prevent ST131 E. coli (i.e., H30R1 and/or H30Rx) colonization and/or infection, to modulate microbial diversity and/or composition, or any combination thereof.

### VII. EXAMPLES

There is thus an urgent need to develop alternative approaches to preventing infections caused by E. coli ST-131 (including ST131-H30R and ST131-H30Rx). Escherichia coli sequence type 131 and its fluoroquinolone resistant H30R subclone lineage (ST131-H30R) is a leading cause of antimicrobial resistant E. coli infections globally. ST131-H30R is commonly carried in the gut as an opportunistic pathogen before subsequent infection of the colonized host.

The Examples that follow are illustrative of specific aspects of the invention, and various uses thereof. They set forth for explanatory purposes only and are not to be taken as limiting the invention.

### Experimental Aims

Escherichia coli (E. coli) sequence type 131 (ST131), especially its recently emerged H30R subclone and the two main clonal subsets within H30R (H30R1 and H30Rx) cause a substantial portion of anti-microbial resistant infections in the U.S. and worldwide. (Johnson J R, et al. (2017) Open Forum Infect Dis. 4: ofx089). Both H30R1 and H30Rx are characterized by fluoroquinolone resistance. Additionally, many H30R strains-especially within H30Rx—are co-resistant to multiple other antimicrobial agents, most notably-advanced beta-lactams due to plasmidic or chromosomal extended-spectrum beta-lactamases and, increasingly, carbapenemases.

Gut colonization by ST131-H30R significantly increases the risk for subsequent infection. (Tchesnokova V L, et al. (2020) Clin Infect Dis. 70:937-939). Individuals can become colonized by antibiotic-resistant E. coli via horizontal transmission, such as through exposures from international travel (Langelier C, et al. (2019) Emerg Infect Dis. 25:1380-1383) within households (Valverde A, et al. (2008) J Clin Microbiol. 46:2796-2799) or in the healthcare setting (Hilty M, et al. (2012) Clin Infect Dis. 55:967-975).

A key feature of ST131-H30R (including the H30R1 and H30Rx subsets) that underlies its pandemic emergence is its ability to stably colonize the human gastrointestinal tract (Tchesnokova V L, et al. (2020) Clin Infect Dis. 70:937-939; Johnson J R, et al. (2022) J Infect Dis. 225:2197-2207). This is mediated, at least in part, through bacterial factors such as FimH, (Sokurenko E V, et al. (1998) Proc Natl Acad Sci USA. 95:8922-8926; Connell I, et al. (1996) Proc Natl Acad Sci USA. 93:9827-9832), fluoroquinolone resistance (Johnson J R, et al. (2012) Antimicrob Agents Chemother. 56:2364-2370), and accessory traits such as iron uptake systems and protectins (Johnson J R, et al. (2022) J Infect Dis. 225:2197-2207). However, not all individuals exposed to known carriers of ST131-H30R become colonized (Johnson J R, et al. (2016) Open Forum Infect Dis. 3 (3): ofw129; Torres E, et al. (2018) Clin Microbiol Infect. 24:618-623).

Studies of probiotics have found that certain gut microbiome features are associated with failed probiotic engraftment, indicating that microbiome composition can determine whether colonization occurs after exposure to a new bacterium. (Zmora N, et al. (2018) Cell. 174:1388-1405.e21). This potential microbiome-mediated resistance to gut bacterial colonization may extend to opportunistic pathogens such as ST131-H30R, which if true could lead to probiotic solutions to protect against ST131-H30R colonization. Previous studies, including a longitudinal study of 27 Dutch nursing home residents, (Ducarmon Q R, et al. (2021) Genome Med. 13:54) have examined the association of gut microbiome composition and host colonization by fluoroquinolone-resistant E. coli (Liss M A, et al. (2019) Investig Clin Urol. 60:75-83) or multidrug-resistant organisms. (Araos R, et al. (2019) Front Microbiol. 10:2260). However, the influence of gut microbiome on gut colonization by the major pandemic subclone ST131-H30R is unknown.

Here, the primary study objective was to determine how the gut microbiome correlates with carriage, persistence, and loss of ST131-H30R. To this end, members of households having at least one individual carrying fluoroquinolone-resistant E. coli and/or ST131 were identified and characterized. Using the combination of a cross-sectional study of 519 participants and a nested longitudinal household study of 141 participants, how the gut microbiome composition correlated with the temporal dynamics of ST131 carriage was determined.

### Materials and Methods

Fecal swabs from consenting subjects were collected according to a protocol approved by the University of Minnesota Institutional Review Board (1412M58401), Minneapolis VA Health Care System IRB (4490-A), and George Washington University IRB (121508). As described elsewhere (Johnson J R, et al. (2022) J Infect Dis. 225:2197-2207), study subjects included military veterans under care at the Minneapolis Veterans Affairs Medical Center (MVAMC) and their household members. Veterans were recruited prospectively (2014 through 2018) by sending invitations for study participation to all newly discharged MVAMC inpatients and randomly selected outpatients. Veterans who agreed to participate were encouraged to refer all available adult and child household members and pets. Swabs were mailed to the research laboratory at room temperature in commercial transport medium, along with basic demographic information. Other clinical data were not obtained.

To assess persistent ST131 carriage, subjects whose initial fecal swab yielded fluoroquinolone-resistant E. coli and/or ST131 (as detected using the below described methods) were offered serial fecal sampling, along with their household members. Serial sampling was done monthly for six months, then every three months, until the first of the following occurred: the end of the study period, the subject/household declined further follow-up, or on two consecutive sampling occasions no household member yielded the household's initial strain of interest.

### Culture Methods

In the research laboratory, fecal swabs provided by study participants were screened for fluoroquinolone-resistant E. coli by selective culture (using Tergitol-7 agar plates with and without ciprofloxacin 4 μg/mL), for overnight incubation at 37 °C. Individual colonies of presumptive E. coli—i.e., lactose-positive colonies with a characteristic E. coli morphology-were assessed for species identity using indole and citrate phenotype (indole positive, citrate-negative).

Molecular Characterization of E. coli Isolates

Standard protocols were used for detection of fluoroquinolone-resistant ST131-H30 isolates using highly sensitive and specific established PCR-based assays, as previously reported (Johnson J R, et al. (2022) J Infect Dis. 225:2197-2207).

In brief, up to 10 presumptive E. coli colonies per plate were screened for clonality by using random amplified polymorphic DNA (RAPD) analysis. Using duplicate boiled lysates for template DNA and relevant positive and negative controls, one representative colony per unique RAPD profile per sample was screened for ST131 by PCR and ST131 isolates were screened to identify the H30 and H30Rx subclones. Fluoroquinolone-resistant ST131-H30 isolates were classified as ST131-H30R, and H30R isolates that tested negative for H30Rx were classified as H30R1.

### ST131-H30R Carriage Pattern Categorization

Based on their E. coli strain carriage pattern, participants were assigned to one of five categories. These included: (i) sustained positive (i.e., participants with ST131-H30R at all time points); (ii) exposed, sustained negative (i.e., participants exposed to a ST131-H30R positive household member but who remained ST131-H30R negative); (iii) sustained loss (i.e., participants with at least one ST131-H30R isolate, followed by at least two samplings without ST131-H30R, and who never re-acquired ST131-H30R); (iv) unexposed negative (i.e., participants without ST131-H30R and not exposed to a household member with ST131-H30R); and (v) other (i.e., participants not falling into the above four categories).

### Sample Processing and Microbiome Characterization

DNA isolation and purification were performed using the collected fecal swabs. Total DNA was extracted from 200 μl of undiluted swab eluent using the MagAttract PowerMicrobiome DNA/RNA kit (Qiagen Inc., Valencia, CA, USA) and eluted into 100 μL of elution buffer and stored at −80 ° C until analysis.

The prevalence and proportional abundance of specific bacterial taxa in each fecal swab were characterized by sequencing the 16S rRNA gene V3-V4 region as described in Fadrosh et al. (Microbiome (2014) 2:6) using MiSeq Reagent Kit v3 (600-cycle) (Illumina Inc., San Diego, CA). A negative extraction control (NEC) was included with each batch of extraction and sequenced to assess for cross contamination. No-template controls (NTCs) and positive-template controls (PTCs) were included to assess for cross-contamination and verify PCR performance.

An in-house pipeline built with published tools was used for amplicon data processing. Briefly, primers were clipped by cutadapt v2.4 (Mohamed M, et al. (2016) Antimicrob Agents Chemother. 60:4638-4645) and resultant sequences quality-trimmed using Trimmomatic v0.39 (Fadrosh D W, et al. (2014) Microbiome. 2:6) and then processed with DADA2 v1.10 (Martin M. (2011) EMBnet.journal. 17:10) to identify amplicon sequence variants (ASVs). ASVs were queried using BLAST v.2.2.30 (Bolger A M, et al. (2014) Bioinformatics. 30:2114-2120) with cut off values of 99% nucleotide identity and 100% query coverage to remove bloomed ASVs (Callahan B J, et al. (2016) Nat Methods. 13:581-583) (available at https://github.com/knightlab-analyses/bloomanalyses/blob/master/data/newbloom.all.fna). After removing bloom ASVs, the remaining ASVs were classified at 80% bootstrap confidence level using the Naïve Bayesian Classifier (v.2.12). (Altschul S F, et al. (1990) J Mol Biol. 215:403-410). Additional details can be found at https://github.com/araclab/mb_analysis.

Samples with under 1000 reads were excluded from analysis. After filtering out rare taxa (any genera accounting for <0.0025% of the entire dataset), the resultant sequencing data were used to calculate the prevalence and the proportional abundance of each taxon for each sample (i.e., number of 16S rRNA gene sequences assigned to a taxon divided by the total number of 16S rRNA sequences). Data from this study is available at SRA project number PRJNA838578

### Comparative Gut Microbiome Analysis

Cross-sectional data was used to identify gut microbiome features that are associated with ST131-H30R carriage, then used longitudinal data to determine how the identified gut microbiome features correlate with ST131-H30R carriage and loss. Samples from pets were excluded from all analyses. Primary analyses focused on comparing ST131-negative participants to H30R-positive participants, given the clinical ambiguity of non-H30R ST131 strain carriage. Supplemental analyses included comparisons across four mutually exclusive carriage subcategories (ST131-negative, non-H30R ST131, ST131-H30R1, ST131-H30Rx).

The cross-sectional study included, for ST131 carriers, the first ST131-positive sample and, for ST131 non-carriers, the first sample overall, which by definition was ST131-negative. Alpha-diversity was measured using the Shannon diversity index. Among bacterial phyla with ≥10% prevalence (i.e., proportion of subjects positive), phyla associated with ST131-H30R carriage were identified using analyses as described below.

Longitudinal data was used to identify gut bacterial taxa associated with ST131-H30R carriage. For this, three sets of candidate genera were considered. The first set comprised genera that were members of phyla identified as being associated with ST131-H30R carriage in the cross-sectional study and also presented in at least 25% of the overall study population. The second set comprised the 10 most-informative genera, as identified by using the mean decrease in Gini values in conjunction with the largest number of inclusions in trees in random forest models. The third set comprised two genera that were regarded a priori as being of interest: Bacteroides and Escherichia/Shigella. For the random forest model, a breakpoint was selected for the number of taxa to be included by identifying an elbow on a scree plot based on the Gini and margin values.

Chi-squared tests and Wilcoxon Rank-Sum tests were used to identify associations between ST131-H30R carriage and the prevalence and proportional abundance of phyla and genera. Correlations between ST131-H30R carriage and the proportional abundance of phyla and genera were evaluated among longitudinal samples using a mixed effects logistic regression model, allowing for repeated measures by individual. Statistical analyses were conducted in SAS, version 9.4, and R, version 3.3.1.

### Example 1

### Characterization of Participant Demographics and ST131 Carriage

Of the 519 participants, 62.8% were veterans, 31.6% were a veteran's spouse, and 5.6% were a child or non-spouse adult in the household (Table 1). The initial fecal screening by conventional culture and PCR identified 78 (15.0%) participants as carriers of ST131 and/or fluoroquinolone-resistant E. coli. All members of households with an ST131 E. coli carrier were invited to undergo longitudinal surveillance; an average of 1.6 participants were enrolled per household. In total, 141 participants-including 44 of 49 H30R-positive participants (mean, 8.5 samples each [SD=3.8]) and 73 of 441 ST131-negative participants (mean, 5.5 samples each [SD=2.3])—were followed longitudinally.

TABLE 1
Demographic and Other Characteristics of Participants

| | No. (column %) | | | | | | | |
|---|---|---|---|---|---|---|---|---|
| | | | | | ST131 | | | |
| | Non-ST131 | | Any ST131 | | Non-H30R | | H30R | |
| Baseline Characteristics | n = 441 | | n = 78 | | n = 29 | | n = 49 | |
| Participant | | | | | | | | |
| Veteran (patient) | 279 | (63.3%) | 47 | (60.3%) | 18 | (62.1%) | 29 | (59.2%) |
| Child | 17 | (3.9%) | 2 | (2.6%) | 1 | (3.5%) | 1 | (2.0%) |
| Spouse | 136 | (30.8%) | 28 | (35.9%) | 10 | (34.5%) | 18 | (36.7%) |
| Other Adult | 9 | (2.0%) | 1 | (1.3%) | 0 | (—) | 1 | (2.0%) |
| Patient Type | | | | | | | | |
| Inpatient | 186 | (42.2%) | 25 | (32.1%) | 14 | (48.3%) | 12 | (24.5%) |
| Outpatient | 255 | (57.8%) | 53 | (68.0%) | 15 | (51.7%) | 37 | (75.5%) |
| Longitudinal Visits | | | | | | | | |
| ≥2 longitudinal visits | 84 | (19.0%) | 57 | (73.1%) | 22 | (75.9%) | 36 | (73.5%) |

### Example 2

Characterization of Prevalence and Dynamics of Intestinal E. coli and ST131

According to genus-level sequencing data, at baseline 463 (89.2%) of the 519 study participants carried Escherichia. The proportional abundance of Escherichia at baseline ranged from 0% to 92.1% (mean 18.8%, SD 19.1) among the 519 participants.

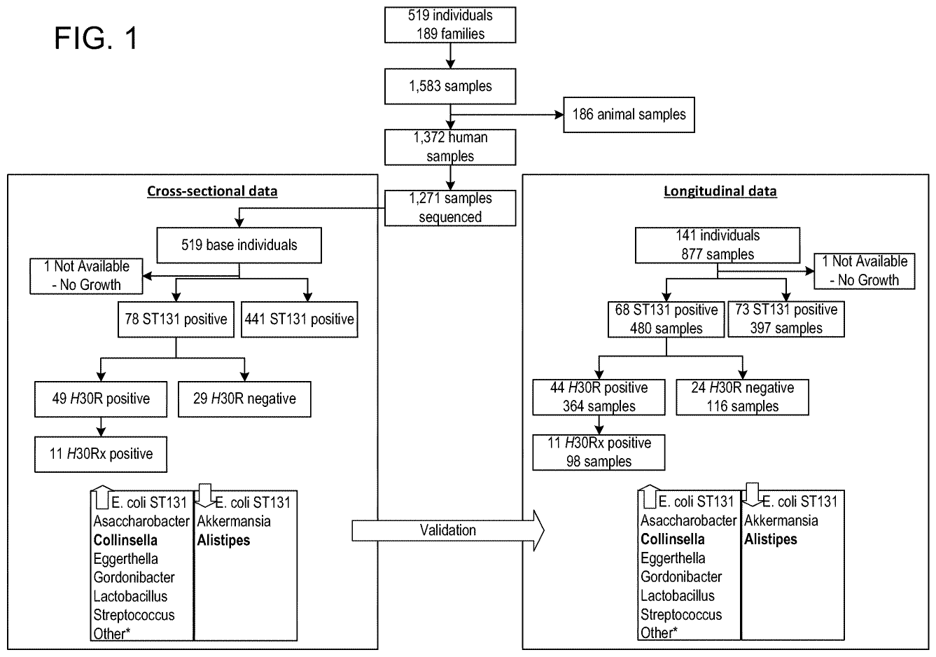

According to PCR testing, 78 (15.0%) of the 519 participants had ST131 in one or more samples, including 49 (9.2%) with H30R, among which 38 (7.1%) were H30R1 and 11 (2.1%) were H30Rx (FIG. 1). Of the 141 participants with longitudinal data, 25 (18%) were persistently H30R-positive (i.e., sustained positives), 11 (8%) were persistently H30R-negative despite household exposure (i.e., exposed, sustained negatives), 14 (10%) were H30R-positive, then persistently H30R-negative (i.e., sustained loss), 62 (44%) were H30R-negative in the absence of household exposure (unexposed negatives), and 27 (19%) exhibited other patterns.

### Example 3

Cross-Sectional Analysis for Gut Microbiome Characteristics Associated with ST131-H30R Carriage

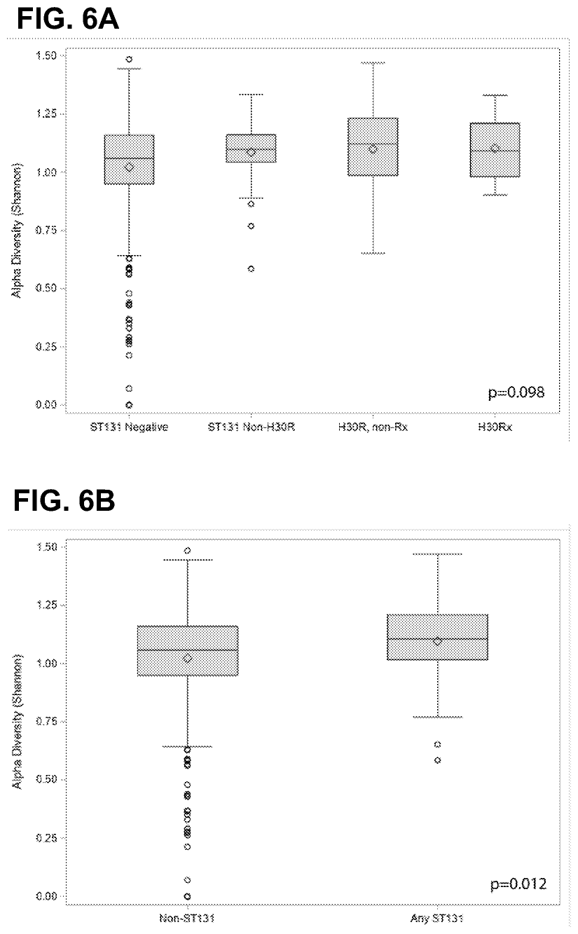

To identify gut bacterial taxa associated with carriage of ST131-H30R, a cross-sectional analysis involving all 519 participants was performed, focusing initially on phyla, then genera. Alpha diversity was similar across all ST131 sub-categories (non-H30R, H30R, and H30Rx; p=0.95), but was significantly lower when comparing ST131-negative individuals compared with ST131-positive individuals (p=0.012, FIG. 6A-FIG. 6B).

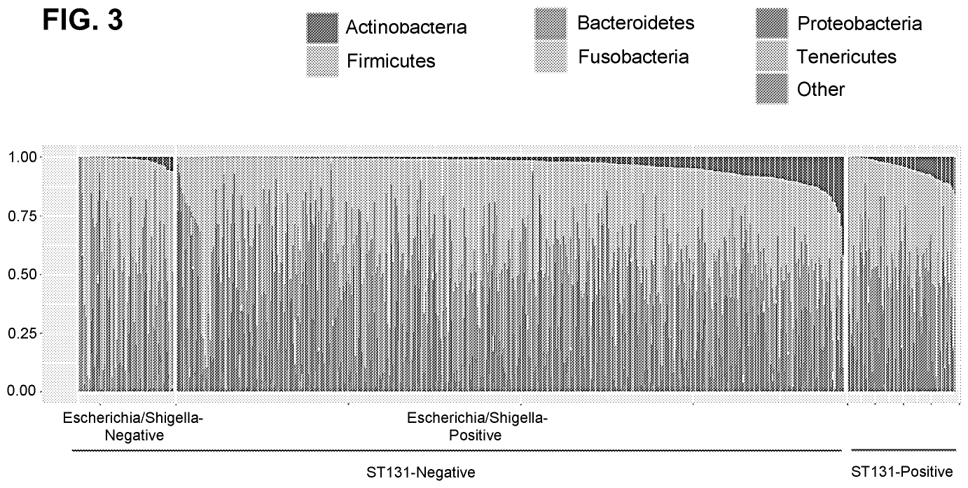

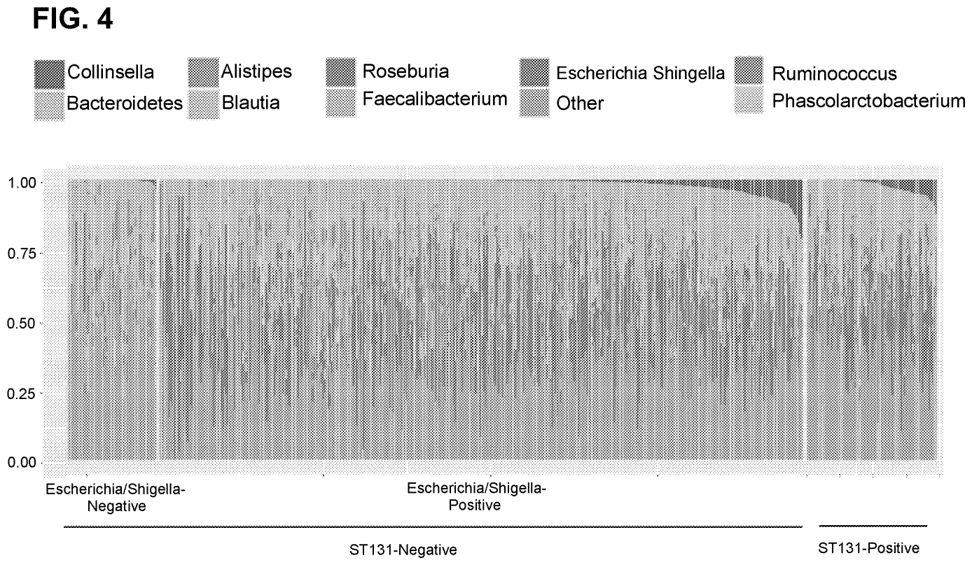

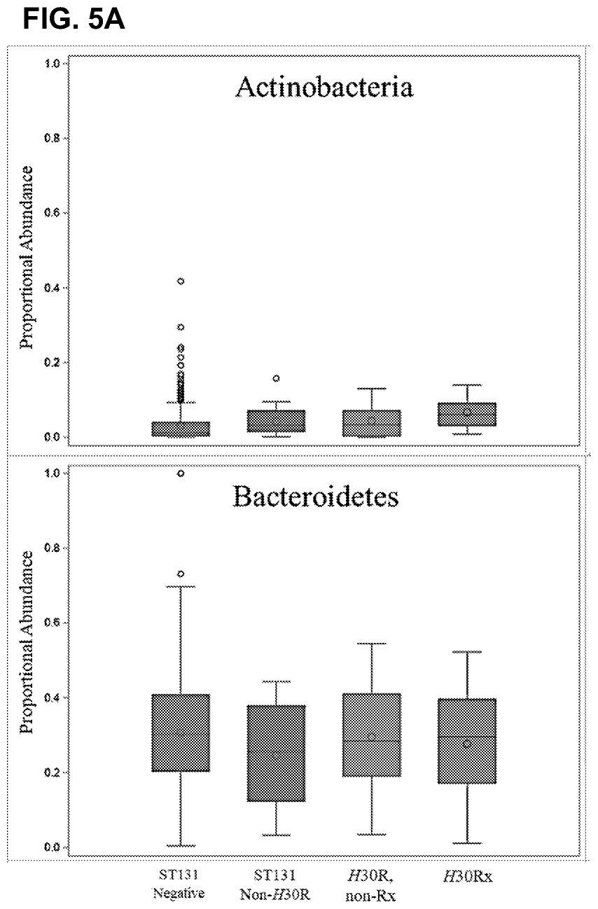

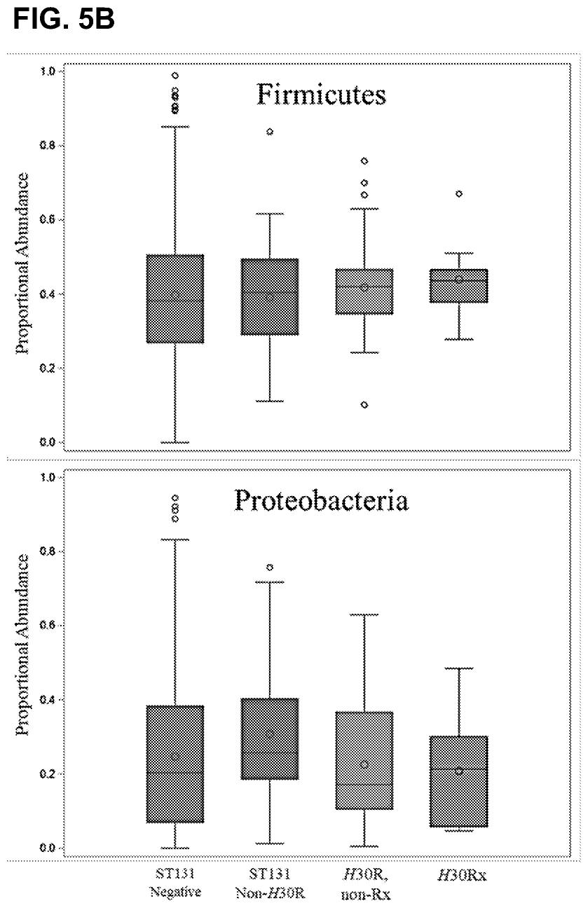

At the phylum level, ST131-H30R carriage was associated with both a higher proportional abundance of Actinobacteria and, conversely, a lower proportional abundance of Verrucomicrobia. Specifically, H30R-positive participants had a significantly higher proportional abundance of Actinobacteria (mean 4.9%, SD 4.1), as compared to ST131-negative participants (mean 3.1%, SD 4.6) (p=0.002) (Table 2). Additionally, H30Rx-positive participants had the highest abundance of Actinobacteria (mean 6.7%, SD 4.0), whereas participants with no detectable Escherichia had the lowest abundance (mean 1.3%, SD 1.6) (Table 3, FIG. 3). By contrast, H30R-positive participants had a significantly lower proportional abundance of Verrucomicrobia (mean 0.3%, SD 1.0) than did ST131-negative participants (mean 1.3%, SD 3.4) (p=0.04).

TABLE 2A
Prevalence and Mean Proportional Abundance of Genera in Cross-Sectional
Analysis.

| | | Prevalence^{b}, Column Percent | | | | | |
|---|---|---|---|---|---|---|---|
| | | | | C. | | | |
| | | A. | B. | ST131, | D. | | |
| | | non- | any- | non- | ST131, | p value | |
| | Random | ST131 | ST131 | H30R | H30R | A. vs | A. vs |
| Phylum or Genus | Forest^{a} | N = 441 | N = 78 | N = 29 | N = 49 | B. | D. |
| Actinobacteria | | 94 | 96 | 100 | 94 | 0.51 | 0.90 |
| Asaccharobacter^{d} | X | 25 | 46 | 48 | 45 | <0.001 | 0.004 |
| Bifidobacterium | | 62 | 71 | 72 | 69 | 0.15 | 0.30 |
| Collinsella | X | 67 | 82 | 90 | 78 | 0.007 | 0.12 |
| Eggerthella | X | 47 | 62 | 72 | 55 | 0.02 | 0.31 |
| Gardnerella | X | 7 | 26 | 38 | 18 | <0.001 | 0.006 |
| Gordonibacter | | 32 | 54 | 55 | 53 | <0.001 | 0.003 |
| Bacteroidetes | | 100 | 100 | 100 | 100 | 1.00 | 1.00 |
| Alistipes | X | 88 | 82 | 90 | 78 | 0.15 | 0.04 |
| Bacteroides | | 100 | 100 | 100 | 100 | 0.55 | 0.64 |
| Firmicutes | | 100 | 100 | 100 | 100 | 0.55 | 0.64 |
| Enterococcus | X | 43 | 53 | 55 | 51 | 0.10 | 0.26 |
| Lactobacillus | X | 33 | 65 | 62 | 67 | <0.001 | <0.001 |
| Streptococcus | X | 68 | 88 | 97 | 84 | <0.001 | 0.02 |
| Weissella | X | 2 | 9 | 14 | 6 | 0.002 | 0.11 |
| Fusobacteria | | 13 | 14 | 14 | 14 | 0.78 | 0.79 |
| Lentisphaerae | | 10 | 5 | 0 | 8 | 0.16 | 0.65 |
| Proteobacteria | | 98 | 100 | 100 | 100 | 0.20 | 0.31 |
| Escherichia | | 88 | 99 | 100 | 98 | 0.003 | 0.03 |
| Oxalobacter^{d} | X | 4 | 12 | 3 | 16 | 0.004 | <0.001 |
| Synergistetes | | 13 | 13 | 0 | 20 | 0.98 | 0.15 |
| Verrucomicrobia | | 52 | 38 | 34 | 41 | 0.048 | 0.22 |
| Akkermansia | | 52 | 38 | 34 | 41 | 0.03 | 0.14 |

Bold denotes p < 0.05.
^{a}X indicates the taxon was selected as one of the top-10 most informative genera for predicting ST131 carriage using the random forest model.
^{b}Percent of participants in a group with a given taxon.
^{c}Mean proportional contribution of each taxon to each participant's gut microbiome (proportional abundance for a given sample was calculated as the number of reads of the given taxon divided by the total number of reads in that sample).
^{d}A genetic near neighbor of the taxon.

TABLE 2B
Mean Proportional Abundance of Genera in Cross-Sectional Analysis.

| | Proportional abundance^{c}, mean (SD) | | | | | |
|---|---|---|---|---|---|---|
| | A. | B. | C. | D. | | |
| | non- | any | ST131, | ST131, | p value | |
| | ST131 | ST131 | non-H30R | H30R | A. | A. |
| Phylum or Genus | N = 441 | N = 78 | N = 29 | N = 49 | vs B. | vs D. |
| Actinobacteria | 3.1(4.6) | 4.7(3.9) | 4.3(3.6) | 4.9(4.1) | <0.001 | 0.001 |
| Asaccharobacter^{d} | 0.1(0.1) | 0.1(0.2) | 0.1(0.2) | 0.1(0.2) | <0.001 | 0.008 |
| Bifidobacterium | 0.9(2.4) | 0.8(1.2) | 0.8(1.2) | 0.7(1.3) | 0.32 | 0.78 |
| Collinsella | 1.1(2.5) | 2.1(2.5) | 1.9(2.3) | 2.3(2.7) | <0.001 | 0.001 |
| Eggerthella | 0.2(0.4) | 0.6(1.1) | 0.7(1.0) | 0.6(1.2) | 0.001 | 0.18 |
| Gardnerella | 0.0(0.3) | 0.2(1.0) | 0.1(0.3) | 0.3(1.3) | <0.001 | 0.004 |
| Gordonibacter | 0.3(1.0) | 0.5(1.2) | 0.5(1.2) | 0.5(1.2) | <0.001 | 0.01 |
| Bacteroidetes | 30.5(15.7) | 27.6(13.0) | 24.5(13.0) | 29.4(12.8) | 0.20 | 0.78 |
| Alistipes | 2.6(3.8) | 1.8(2.9) | 1.2(1.4) | 2.1(3.5) | 0.01 | 0.08 |
| Bacteroides | 23.9(14.7) | 22.2(11.8) | 20.4(11.3) | 23.3(12.1) | 0.52 | 0.78 |
| Firmicutes | 39.7(18.3) | 40.9(14.3) | 39.2(16.5) | 41.9(12.9) | 0.35 | 0.22 |
| Enterococcus | 0.8(3.1) | 2.8(8.0) | 4.5(10.5) | 1.9(6.0) | 0.03 | 0.17 |
| Lactobacillus | 0.3(1.2) | 1.3(3.3) | 1.0(3.0) | 1.5(3.5) | <0.001 | <0.001 |
| Streptococcus | 1.2(3.9) | 2.0(4.5) | 1.5(2.0) | 2.3(5.4) | <0.001 | 0.001 |
| Weissella | 0.0(0.3) | 0.0(0.2) | 0.0(0.1) | 0.0(0.2) | 0.003 | 0.10 |
| Fusobacteria | 0.2(1.2) | 0.1(0.8) | 0.1(0.2) | 0.2(1.0) | 0.87 | 0.85 |
| Lentisphaerae | 0.0(0.1) | 0.0(0.0) | — | 0.0(0.0) | 0.14 | 0.61 |
| Proteobacteria | 24.8(20.4) | 25.5(17.5) | 31.3(18.6) | 22.0(16.0) | 0.35 | 0.74 |
| Escherichia | 18.7(19.6) | 19.5(16.0) | 22.8(16.8) | 17.6(15.4) | 0.07 | 0.37 |
| Oxalobacter^{d} | 0.0(0.0) | 0.0(0.0) | 0.0(0.0) | 0.0(0.0) | 0.004 | <0.001 |
| Synergistetes | 0.1(0.3) | 0.4(3.0) | — | 0.6(3.7) | 0.95 | 0.16 |
| Verrucomicrobia | 1.3(3.4) | 0.3(0.9) | 0.2(0.8) | 0.3(1.0) | 0.003 | 0.04 |
| Akkermansia | 1.3(3.4) | 0.3(0.9) | 0.2(0.8) | 0.3(1.0) | 0.005 | 0.02 |

Bold denotes p < 0.05.
^{a}X indicates the taxon was selected as one of the top-10 most informative genera for predicting ST131 carriage using the random forest model.
^{b}Percent of participants in a group with a given taxon.
^{c}Mean proportional contribution of each taxon to each participant's gut microbiome (proportional abundance for a given sample was calculated as the number of reads of the given taxon divided by the total number of reads in that sample).
^{d}A genetic near neighbor of the taxon.

By contrast with Actinobacteria and Verrucomicrobia, the phyla Bacteroidetes, Firmicutes, and Proteobacteria occurred in nearly all participants, and did not differ significantly in prevalence or abundance by ST131 status or ST131 subclone carriage. Firmicutes was the most proportionally abundant of these phyla (mean 39.9%, SD 17.8), followed by Bacteroidetes (mean 30.1%, SD 15.4) and Proteobacteria (mean 24.9%, SD 20.0).

TABLE 3A
Prevalence of genera in cross-sectional analysis, stratified
by Escherichia and ST131 subclone categories.

| | Prevalence^{a}, percent | | | | |
|---|---|---|---|---|---|
| | Non-ST131 | | ST131-positive | | |
| | Escherichia | | ST131 | H30R, | |
| | Negative | Positive | non-H30R | non-Rx | H30Rx |
| Phylum or genus | n = 55 | n = 386 | n = 29 | n = 38 | n = 11 |
| Actinobacteria | 87.3 | 95.3 | 100 | 92.1 | 100 |
| Asaccharobacter | 5.5 | 28.2 | 48.3 | 42.1 | 54.6 |
| Bifidobacterium | 52.7 | 63.2 | 72.4 | 65.8 | 81.8 |
| Collinsella | 41.8 | 70.2 | 89.7 | 71.1 | 100 |
| Eggerthella | 47.3 | 47.4 | 72.4 | 55.3 | 54.6 |
| Gardnerella | 1.8 | 7.8 | 37.9 | 18.4 | 18.2 |
| Gordonibacter | 20.0 | 33.7 | 55.2 | 50.0 | 63.6 |
| Bacteroidetes | 100 | 100 | 100 | 100 | 100 |
| Alistipes | 87.3 | 88.1 | 89.7 | 76.3 | 81.8 |
| Bacteroides | 98.2 | 99.7 | 100 | 100 | 100 |
| Firmicutes | 96.4 | 100 | 100 | 100 | 100 |
| Enterococcus | 38.2 | 43.3 | 55.2 | 52.6 | 45.5 |
| Lactobacillus | 18.2 | 35.5 | 62.1 | 68.4 | 63.6 |
| Streptococcus | 60.0 | 68.7 | 96.6 | 81.6 | 90.9 |
| Weissella | 1.8 | 2.3 | 13.8 | 7.9 | 0 |
| Fusobacteria | 7.3 | 13.7 | 13.8 | 13.2 | 18.2 |
| Lentisphaerae | 5.5 | 10.9 | 0 | 5.3 | 18.2 |
| Proteobacteria | 83.6 | 100 | 100 | 100 | 100 |
| Escherichia | 0 | 100 | 100 | 100 | 90.9 |
| Oxalobacter* | 1.8 | 4.2 | 3.5 | 18.4 | 9.1 |
| Synergistetes | 12.7 | 13.0 | 0 | 15.8 | 36.4 |
| Verrucomicrobia | 41.8 | 53.4 | 34.5 | 42.1 | 36.4 |
| Akkermansia | 41.8 | 53.4 | 34.5 | 42.1 | 36.4 |

*Genetic near-neighbor of the given taxon
^{a}percent of participants in a group with a given taxon
^{b} mean proportional contribution of each taxa to the gut microbiome of each participant, averaged across each group. PerMANOVA comparing proportional abundance at the phylum level across groups (Non-ST131, ST131 non-H30R, H30R non-Rx, H30Rx), p value = 0.158

TABLE 3B
Mean proportional abundance of genera in cross-sectional analysis,
stratified by Escherichia and ST131 subclone categories.

| | Proportional Abundance^{b}, mean (SD) | | | | | | | | | |
|---|---|---|---|---|---|---|---|---|---|---|
| | Non-ST131 | | | | ST131-positive | | | | | |
| | Escherichia | | | | ST131 | | H30R, | | | |
| | Negative | | Positive | | non-H30R | | non-Rx | | H30Rx | |
| Phylum or genus | n = 55 | | n = 386 | | n = 29 | | n = 38 | | n = 11 | |
| Actinobacteria | 1.3 | (1.6) | 3.3 | (4.8) | 4.3 | (3.6) | 4.4 | (4.1) | 6.7 | (4.0) |
| Asaccharobacter | 0.0 | (0.0) | 0.1 | (0.1) | 0.1 | (0.2) | 0.1 | (0.2) | 0.2 | (0.2) |
| Bifidobacterium | 0.6 | (1.3) | 1.0 | (2.5) | 0.8 | (1.2) | 0.7 | (1.3) | 0.8 | (1.0) |
| Collinsella | 0.2 | (0.6) | 1.3 | (2.7) | 1.9 | (2.3) | 1.7 | (2.2) | 4.1 | (3.4) |
| Eggerthella | 0.2 | (0.3) | 0.2 | (0.4) | 0.7 | (1.0) | 0.7 | (1.3) | 0.2 | (0.4) |
| Gardnerella | 0.0 | (0.0) | 0.0 | (0.3) | 0.1 | (0.3) | 0.2 | (1.2) | 0.5 | (1.5) |
| Gordonibacter | 0.0 | (0.1) | 0.3 | (1.1) | 0.5 | (1.2) | 0.6 | (1.4) | 0.4 | (0.6) |
| Bacteroidetes | 37.2 | (21.9) | 29.6 | (14.4) | 24.5 | (13.0) | 29.7 | (12.2) | 28.2 | (15.4) |
| Alistipes | 3.7 | (4.1) | 2.4 | (3.7) | 1.3 | (1.4) | 2.0 | (3.7) | 2.5 | (2.5) |
| Bacteroides | 28.4 | (21.9) | 23.3 | (13.3) | 20.4 | (11.3) | 23.9 | (12.3) | 21.5 | (11.8) |
| Firmicutes | 45.6 | (26.1) | 38.9 | (16.8) | 39.2 | (16.5) | 41.0 | (13.5) | 45.0 | (10.5) |
| Enterococcus | 1.5 | (4.5) | 0.7 | (2.8) | 4.5 | (10.5) | 2.3 | (6.8) | 0.4 | (0.7) |
| Lactobacillus | 0.2 | (1.0) | 0.3 | (1.2) | 1.0 | (3.0) | 1.7 | (3.9) | 0.5 | (1.1) |
| Streptococcus | 1.4 | (3.0) | 1.2 | (4.0) | 1.5 | (2.0) | 1.1 | (1.9) | 6.4 | (10.2) |
| Weissella | 0.1 | (0.6) | 0.0 | (0.2) | 0.0 | (0.1) | 0.0 | (0.2) | — | |
| Fusobacteria | 0.1 | (0.6) | 0.2 | (1.3) | 0.1 | (0.2) | 0.2 | (1.1) | 0.0 | (0.0) |
| Lentisphaerae | 0.0 | (0.0) | 0.0 | (0.1) | — | | 0.0 | (0.0) | 0.0 | (0.0) |
| Proteobacteria | 13.8 | (19.5) | 26.3 | (20.0) | 31.3 | (18.6) | 22.9 | (16.4) | 19.1 | (15.0) |
| Escherichia | — | | 21.3 | (19.6) | 22.8 | (16.8) | 17.7 | (15.4) | 17.4 | (15.9) |
| Oxalobacter* | 0.0 | | 0.0 | | 0.0 | | 0.0 | | 0.0 | |
| Synergistetes | 0.1 | (0.3) | 0.1 | (0.3) | — | | 0.7 | (4.2) | 0.2 | (0.8) |
| Verrucomicrobia | 2.0 | (4.1) | 1.2 | (3.3) | 0.2 | (0.8) | 0.4 | (1.1) | 0.1 | (0.2) |
| Akkermansia | 2.0 | (4.1) | 1.2 | (3.3) | 0.2 | (0.8) | 0.4 | (1.1) | 0.1 | (0.2) |

*Genetic near-neighbor of the given taxon
^{a} percent of participants in a group with a given taxon
^{b}mean proportional contribution of each taxa to the gut microbiome of each participant, averaged across each group. PerMANOVA comparing proportional abundance at the phylum level across groups (Non-ST131, ST131 non-H30R, H30R non-Rx, H30Rx), p value = 0.158

### Example 4

Cross-Sectional Analysis for Genera and Species Associated with ST131-H30R Carriage

At the genus level, although multiple genera were positively associated with H30R carriage, only Alistipes was negatively associated with H30R carriage. Specifically, of the genera examined, Gardnerella, Gordonibacter, Lactobacillus, Streptococcus, and a phylogenetic near neighbor of Asacharobacter all exhibited a higher prevalence and greater proportional abundances in association with carriage of ST131 and H30R, and Collinsella, Eggerthella, and Weissella exhibited similar association with carriage of ST131 (but not H30R).

By contrast, Alistipes and Akkermansia were negatively associated with H30R carriage. Specifically, as compared with H30R-positive participants, ST131-negative participants had a higher prevalence or proportional abundance of Alistipes (p=0.04 and p=0.08, respectively), and a higher proportional abundance of Akkermansia (p=0.02), although a similar prevalence of Akkermansia (p=0.14). Species-level associations are provided in supplemental analyses for Alistipes, along with the three most-prevalent genera associated with ST131 carriage (Collinsella, Lactobacillus, Streptococcus) (Table 5A and Table 5B).

### Example 5

### Longitudinal Associations Between Genera and H30R Carriage

Next, a longitudinal analyses of the gut microbiome was performed and was limited to the genus level, from 25 participants with either of two clinically relevant H30R colonization patterns: exposed, sustained negative (participants with a H30R-positive household member who remained H30R-negative for the duration of the study) and sustained loss (H30R-positive individuals who became H30R-negative during the study). On average, participants contributed 7.6 visits.

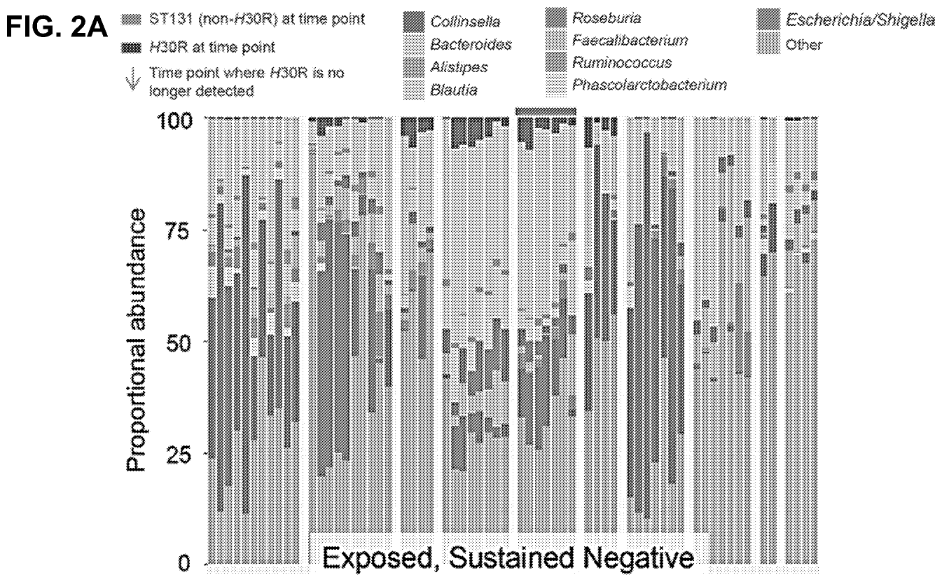

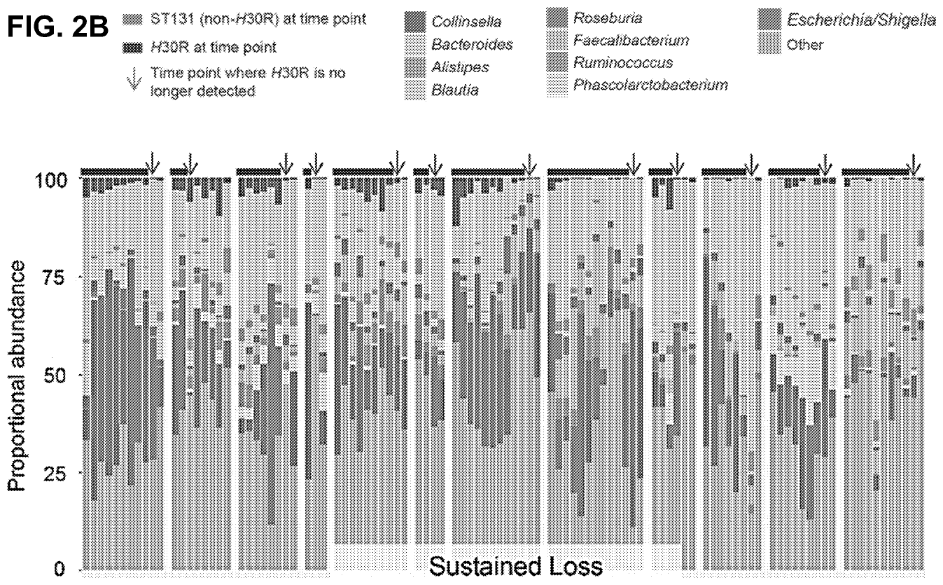

Findings from the cross-sectional analysis were corroborated in the longitudinal analysis by using a mixed-effects logistic regression model that controlled for intra-individual variation over time and included all genera of interest from Table 2. Alistipes was the only genus significantly associated with absence of H30R (p=0.005), whereas Collinsella was the only genus associated with presence of H30R (p=0.035) (Table 4). No other taxa displayed significant associations with H30R carriage. Among participants with sustained loss of H30R, Collinsella decreased in median proportional abundance with loss of H30R, from 1.5% to 0.5% (FIG. 2A-FIG. 2B). Conversely, median proportional abundance of Alistipes increased from 0.8% to 2.0% after loss of H30R. Collinsella and Alistipes median proportional abundance were both low (0.2% and 0.4%, respectively) for participants who were exposed to a H30R-positive family member but remained ST131-negative.

TABLE 4
Genera Associated with H30R Carriage Compared with ST131-Negative
Status Across Longitudinally Collected Samples from Exposed
Sustained Negative and Sustained Loss Participants.

| | Model | Std. | t | Probability > |
|---|---|---|---|---|
| | Estimate | Error | value | \|t\| |
| Actinobacteria | | | | |
| Asaccharobacter | −109.5 | 188.1 | −0.58 | 0.56 |
| Bifidobacterium | −25.1 | 42.3 | −0.59 | 0.55 |
| Collinsella | 31.7 | 14.9 | 2.13 | 0.035 |
| Eggerthella | 43.6 | 45.1 | 0.97 | 0.34 |
| Gardnerella | −6.6 | 64.4 | −0.10 | 0.92 |
| Gordonibacter | −5.1 | 23.5 | −0.22 | 0.83 |
| Bacteroidetes | | | | |
| Alistipes | −66.3 | 23.4 | −2.83 | 0.005 |
| Bacteroides | 0.4 | 2.7 | 0.15 | 0.89 |
| Firmicutes | | | | |
| Enterococcus | −0.5 | 4.3 | −0.11 | 0.92 |
| Lactobacillus | 4.9 | 22.3 | 0.22 | 0.83 |
| Streptococcus | 37.1 | 23.3 | 1.59 | 0.11 |
| Weissella | −99.1 | 221.6 | −0.45 | 0.66 |
| Proteobacteria | | | | |
| Escherichia | 0.2 | 2.0 | 0.08 | 0.93 |
| Oxalobacter* | 2929.2 | 2474.3 | 1.18 | 0.24 |
| Verrucomicrobia | | | | |
| Akkermansia | 19.5 | 28.9 | 0.67 | 0.50 |

*Genetic near-neighbor of a given taxon
^{a} Evaluated using a mixed effects logistic regression model using taxa proportional abundance

In this study of veterans and their household members, specific phyla and genera in the gut microbiome were consistently associated with ST131 and H30R carriage. In cross-sectional comparisons, the phylum Actinobacteria and certain common genera, including Lactobacillus, Streptococcus, and Collinsella (a member of Actinobacteria), were associated with presence of ST131 and H30R. In longitudinal analyses, the genus Collinsella again was associated with H30R-positivity, whereas the genus Alistipes was associated with absence and loss of ST131 and H30R. These findings identify potential opportunities for influencing gut colonization by ST131 and its H30R subclone.

The H30R subclone of E. coli, which has emerged as an important multidrug-resistant pathogen, causes a significant portion of antimicrobial-resistant infections in the U.S. and worldwide. (Johnson J R, et al. (2017) Open Forum Infect Dis. 4: ofx089; Amir A, et al. (2017) mSystems. 2 (2): e00199-16). Although H30R is known to be an excellent gut colonizer (Johnson J R, et al. (2022) J Infect Dis. 225:2197-2207), the gut microbiome features associated with acquisition, persistence, and loss of H30R carriage are poorly understood. To our knowledge, this study provides the first description of the bacterial phyla and genera associated with H30R gastrointestinal carriage. If the observed associations represent a causal effect of specific microbiota on H30R colonization, interventions that shift those taxa could have important public health benefits.

Collinsella is a member of the phylum Actinobacteria which was associated with presence of ST131 and H30R in both the cross-sectional and longitudinal analyses (specifically, C. aerofaciens; Table 5). Collinsella may be an important health-relevant constituent of the gut microbiome. (Wang Q, et al. (2007) Appl Environ Microbiol. 73:5261-5267). Presence of Collinsella has been associated with diverse diseases, including irritable bowel syndrome, gestational diabetes mellitus, and atherosclerosis, (Nicolas-Chanoine M-H, et al. (2014) Clin Microbiol Rev. 27:543-574; Tourlousse D M, et al. (2020) Microbiol Resour Announc. 9 (16): E00134-20; Kassinen A, et al. (2007) Gastroenterology. 133:24-33), whereas increased abundance of Collinsella aerofaciens has been associated with rheumatoid arthritis. (Karlsson F H, et al. (2012) Nat Commun. 3:1245). Low pro-inflammatory effects, and confer increased susceptibility to impaired glucose tolerance. (Ferrocino I, et al. (2018) Sci Rep. 8:12216). Collinsella aerofaciens has also been linked with increased gut permeability and inflammation, specifically with production of IL-17A, along with high levels of alphaaminoadipic acid and asparagine. (Karlsson F H, et al. (2012) Nat Commun. 3:1245). Conceivably, the associations observed between Collinsella with ST131 and H30R may be due to Collinsella-mediated modulation of the host inflammatory and immune systems in an ST131-favoring manner. If so, this would make Collinsella a potential intervention target. Alternatively, these associations may reflect nutritional or other exposures that jointly favor Collinsella, ST131, and H30R. Identification of such causal factors could have even broader health benefits. Evaluating causal mechanisms may require co-culture models, metagenomic, or meta-transcriptomic approaches.

TABLE 5A
Prevalence of Species in Cross-Sectional Analysis, by Individuals
with Differing Categories of E. coli ST131 and ST131 Subclone
(H30R and Non-H30R) Carriage Status

| | Prevalence^{b}, column percent | | | | | |
|---|---|---|---|---|---|---|
| | A. | | C. | | | |
| | Non- | B. | ST131, | D. | | |
| | ST131 | Any | non- | ST131 | P value | |
| Genus or | N = | ST131 | H30R | H30R | A. | A. |
| species | 441 | N = 78 | N = 29 | N = 49 | vs. B | vs. D. |
| Collinsella | 67 | 82 | 90 | 78 | 0.007 | 0.12 |
| C. aerofaciens | 53 | 79 | 86 | 75 | <0.001 | 0.004 |
| C. intestinalis | 15 | 26 | 31 | 23 | 0.01 | 0.13 |
| C. tanakaei | 4 | 5 | 3 | 6 | 0.52 | 0.38 |
| Alistipes | 88 | 82 | 90 | 78 | 0.15 | 0.04 |
| A. putredinis | 57 | 52 | 48 | 54 | 0.44 | 0.73 |
| A. finegoldii | 34 | 34 | 28 | 38 | 0.99 | 0.60 |
| A. onderdonkii | 49 | 48 | 41 | 52 | 0.92 | 0.66 |
| A. shahii | 63 | 61 | 55 | 65 | 0.72 | 0.85 |
| A. timonensis | 5 | 8 | 3 | 10 | 0.36 | 0.14 |
| Lactobacillus | 33 | 65 | 62 | 67 | <0.001 | <0.001 |
| L. crispatus | 8 | 19 | 28 | 15 | 0.001 | 0.09 |
| L. gasseri | 6 | 14 | 17 | 13 | 0.02 | 0.13 |
| L. reuteri | 3 | 14 | 14 | 15 | <0.001 | <0.001 |
| L. salivarius | 3 | 9 | 7 | 10 | 0.01 | 0.01 |
| L. animalis | 1 | 5 | 7 | 4 | 0.004 | 0.04 |
| L. iners | 11 | 48 | 48 | 48 | <0.001 | <0.001 |
| L. mucosae | 1 | 4 | 7 | 2 | 0.01 | 0.22 |
| Streptococcus | 68 | 88 | 97 | 84 | <0.001 | 0.02 |
| S. anginosus | 9 | 19 | 28 | 15 | 0.01 | 0.20 |
| S. infantarius | 5 | 8 | 14 | 4 | 0.42 | 0.71 |
| S. mitis | 4 | 9 | 7 | 10 | 0.09 | 0.07 |
| S. mutans | 6 | 25 | 21 | 27 | <0.001 | <0.001 |
| S. salivarius | 44 | 75 | 86 | 69 | <0.001 | 0.001 |
| S. sinensis | 18 | 39 | 38 | 40 | <0.001 | 0.001 |

Prevalence and mean proportional abundance of genera and species, by E. coli ST131 carriage status (no ST131, any ST131, and ST131 subsets of non-H30R and any H30R).
Species that had at least 0.1% proportional abundance and 1% overall prevalence were included.

TABLE 5B
Mean Proportional Abundance of Species in Cross-Sectional Analysis,
by Individuals with Differing Categories of E. coli ST131 and
ST131 Subclone (H30R and Non-H30R) Carriage Status

| | Proportional abundance^{c}, mean (SD) | | | | | |
|---|---|---|---|---|---|---|
| | | | C. | | | |
| | A. | B. | ST131 | D. | | |
| | Non- | Any | non-, | ST131 | P value | |
| Genus or | ST131 | ST131 | H30R | H30R | A. | A. |
| species | N = 441 | N = 78 | N = 29 | N = 49 | vs. B | vs. D. |
| Collinsella | 1.1 (2.5) | 2.1 (2.5) | 1.9 (2.3) | 2.3 (2.7) | <0.001 | 0.001 |
| C. aerofaciens | 0.8 (2.1) | 1.8 (2.4) | 1.6 (2.2) | 2 (2.5) | <0.001 | <0.001 |
| C. intestinalis | 0.1 (1.1) | 0.3 (1) | 0.3 (1) | 0.3 (1) | 0.01 | 0.11 |
| C. tanakaei | 0.1 (0.7) | 0 (0.1) | 0.0 (0) | 0.0 (0.2) | 0.52 | 0.38 |
| Alistipes | 2.6 (3.8) | 1.8 (2.9) | 1.2 (1.4) | 2.1 (3.5) | 0.01 | 0.08 |
| A. putredinis | 0.6 (0.9) | 0.5 (1) | 0.4 (0.9) | 0.5 (1.1) | 0.09 | 0.28 |
| A. finegoldii | 0.3 (0.8) | 0.3 (1.1) | 0.1 (0.3) | 0.3 (1.3) | 0.69 | 0.94 |
| A. onderdonkii | 0.9 (2.9) | 0.5 (1.2) | 0.3 (0.6) | 0.6 (1.4) | 0.61 | 0.94 |
| A. shahii | 0.8 (1.6) | 0.5 (0.9) | 0.5 (0.8) | 0.6 | 0.39 | 0.93 |
| | | | | (0 [symbol] | | |
| A. timonensis | 0.1 (0.4) | 0.0 (0.2) | 0.0 (0) | 0.0 (0.2) | 0.40 | 0.16 |
| Lactobacillus | 0.3 (1.2) | 1.3 (3.3) | 1.0 (3.0) | 1.5 (3.5) | <0.001 | <0.001 |
| L. crispatus | 0.0 (0.1) | 0.1 (1) | 0.3 (1.6) | 0.0 (0.2) | 0.001 | 0.10 |
| L. gasseri | 0.0 (0.3) | 0.1 (0.4) | 0.1 (0.2) | 0.1 (0.5) | 0.02 | 0.11 |
| L. reuteri | 0.0 (0.3) | 0.1 (0.2) | 0.0 (0.1) | 0.1 (0.3) | <0.001 | <0.001 |
| L. salivarius | 0.0 (0.2) | 0.1 (0.6) | 0.0 (0) | 0.2 (0.7) | 0.01 | 0.01 |
| L. animalis | 0.0 (0.1) | 0.2 (1.6) | 0.0 (0) | 0.3 (2) | 0.004 | 0.04 |
| L. iners | 0.0 (0.4) | 0.4 (1.7) | 0.4 (1.4) | 0.4 (1.9) | <0.001 | <0.001 |
| L. mucosae | 0.0 (0.2) | 0.0 (0.3) | 0.0 (0.1) | 0.1 (0.4) | 0.01 | 0.22 |
| Streptococcus | 1.2 (3.9) | 2.0 (4.5) | 1.5 (2.0) | 2.3 (5.4) | <0.001 | 0.001 |
| S. anginosus | 0.1 (1.1) | 0.1 (0.2) | 0.1 (0.2) | 0.1 (0.2) | 0.01 | 0.18 |
| S. infantarius | 0.1 (2) | 0.1 (0.7) | 0.2 (1.1) | 0.0 (0) | 0.43 | 0.75 |
| S. mitis | 0.3 (2.7) | 0.6 (3.9) | 0.1 (0.3) | 0.9 (4.9) | 0.09 | 0.08 |
| S. mutans | 0.0 (0.1) | 0.0 (0.1) | 0.0 (0.0) | 0.1 (0.2) | <0.001 | <0.001 |
| S. salivarius | 0.0 (0.2) | 0.1 (0.3) | 0.1 (0.2) | 0.1 (0.4) | <0.001 | <0.001 |
| S. sinensis | 0.5 (1.7) | 0.9 (1.5) | 0.8 (1.3) | 1.0 (1.6) | <0.001 | 0.001 |

Prevalence and mean proportional abundance of genera and species, by E. coli ST131 carriage status (no ST131, any ST131, and ST131 subsets of non-H30R and any H30R).
Species that had at least 0.1% proportional abundance and 1% overall prevalence were included. [symbol]
indicates data missing or illegible when filed

By contrast with Collinsella, the genus Alistipes was negatively associated with H30R prevalence and ST131 proportional abundance. It has contrastingly been implicated as a risk factor for certain conditions, such as hypertension (Chen J, et al. (2016) Genome Med. 8:43), but protective against others, including atrial fibrillation, cardiovascular disease (Gomez-Arango L F, et al. (2018) Gut Microbes. 9:189-201; Kim S, et al. (2018) Clinical Science. 132:701-718; Jie Z, et al. (2017) Nat Commun. 8:845), ulcerative colitis (Zuo K, et al. (2019) GigaScience. 8 (6): giz058), and liver fibrosis (Cui X, et al. (2018) Sci Rep. 8:635). Studies have linked Alistipes with anti-inflammatory effects in the gut. (Dziarski R, et al. (2016) PLOS ONE. 11: e0146162; Campion D, et al. (2019) World J Hepatol. 11:489-512). These anti-inflammatory effects may be mediated by production of the short-chain fatty acids propionate and acetate (Andoh A. (2016) Digestion. 93:176-181; Arpaia N, et al. (2013) Nature. 504:451) or through (Dziarski R, et al. (2016) PLOS ONE. 11: e0146162; Campion D, et al. (2019) World J Hepatol. 11:489-512) other immuno-modulatory mechanisms, leading to reduced inflammation in the gut and other organ systems. (Polansky O, et al. (2016) Appl Environ Microbiol. 82:1569-1576; Oliphant K, et al. (2019) Microbiome. 7 (1): 91). As with Collinsella, hypothesis testing regarding possible inflammation-mediated mechanisms for the observed relationship between Alistipes and H30R may be worthy of future consideration given the tremendous public health importance of H30R colonization.

Interestingly, multiple gut microbiome studies have observed an inverse correlation between Alistipes and both Escherichia and Streptococcus (Gomez-Arango L F, et al. (2018) Gut Microbes. 9:189-201; Jie Z, et al. (2017) Nat Commun. 8:845; Oliphant K, et al. (2019) Microbiome. 7 (1): 91). Conceivably, reductions in Alistipes may contribute independently to both disease pathogenesis and ST131 and H30R carriage. Further evaluations of interactions between these key taxa may have implications for inflammation and disease and may elucidate potential targets for reductions in ST131-H30R colonization such as targeted probiotics and diet modifications.

The consistency of findings between prevalence and proportional abundance measures, and also between cross-sectional and longitudinal analyses, support validity of the findings with regards to both sensitivity of detecting ST131-H30R isolates and also conclusions regarding Alistipes, Collinsella, and other taxa of interest.

In summary, gastrointestinal carriage of ST131, and specifically H30R, a leading antimicrobial-resistant pathogen, is associated with presence and abundance of Collinsella and certain other genera, whereas absence and loss of H30R are associated with Alistipes. If these associations involving the gut microbiota represent causal pathways that reduce ST131 carriage, bacteria negatively associated with ST131 carriage may be useful directly as a probiotic species, or indirectly via a prebiotic or other dietary manipulations that may promote their growth and thereby protect against ST131 colonization. Conversely, bacteria positively associated with ST131 carriage may be potential targets for vaccines or narrow-spectrum therapeutics such as bacteriophages that aim to mitigate an important public health threat.

### Summary of Examples

Using a large cross-sectional study, random forest models were used to identify bacteria associated with ST131-H30R carriage. Participants with ST131-H30R exhibited higher proportional abundance of Actinobacteria and Collinsella. Conversely, Alistipes was more common in participants without ST131-H30R carriage. These gut microbiome patterns were confirmed in an extensive longitudinal study of individuals and their household members, where Collinsella was present more frequently among persistent ST131-H30R carriers, while Alistipes increased in conjunction with loss of ST131-H30R carriage.

These data indicated that multiple gut bacteria corresponded with E. coli ST131-H30R gut carriage, persistence, and loss. Collinsella has been previously linked with gut inflammation and permeability, whereas Alistipes has been linked with anti-inflammatory effects and has been reported as inversely correlated with Escherichia carriage. This study supports these findings using longitudinal analyses, and extends them specifically to ST131-H30R, showing higher Collinsella abundance alongside persistent ST131-H30R carriage, and a significant increase in Alistipes abundance in conjunction with persistent loss of ST131-H30R. These gut microbiome taxa (Alistipes, Collinsella), or the pathways they represent, may provide targets for therapeutic interventions through which to reduce or prevent ST131-H30R carriage, thereby decreasing transmission and infections from this critical multi-drug resistant opportunistic pathogen.

What is claimed is:

1. A method of reducing the degree and/or severity of a drug-resistant E. coli colonization in a subject, the method comprising:
administering to a subject in need thereof a therapeutically effective amount of a composition,
wherein, following the administering step, the composition reduces the proportional abundance of one or more bacteria associated with the carriage and/or persistence of drug-resistant E. coli bacteria, thereby reducing the degree and/or severity of colonization by a drug-resistance E. coli.

2. The method of claim 1, wherein the composition comprises one or more bacteria belonging to the Alistipes genus and/or Akkermansia genus.

3. The method of claim 2, wherein the Alistipes genus comprises Alistipes finegoldii, Alistipes putredinis, Alistipes onderdonkii, Alistipes shahii, Alistipes indistinctus, Alistipes senegalensis, Alistipes timonensis, Alistipes obesi, Alistipes ihumii, Alistipes inops, Alistipes megaguti, Alistipes provencensis, Alistipes massiliensis, or any combination thereof.

4. The method of claim 1, wherein the composition inhibits proliferation of Asaccharobacter, Bifidobacterium, Collinsella, Eggerthella, Gardnerella, Gordonibacter, or any combination thereof in the gut microbiome of the subject.

5. The method of claim 1, wherein the one or more E. coli bacteria comprise sequence type 131 (ST131).

6. The method of claim 5, wherein the one or more ST131 E. coli bacteria comprise a H30R subclone.

7. The method of claim 6, wherein the H30R subclone comprises H30R1 or H30Rx.

8. The method of claim 1, wherein the drug resistant E. coli bacteria are multi-drug resistant.

9. The method of claim 1, wherein the drug resistant E. coli bacteria are fluoroquinolone resistant.

10. The method of claim 1, further comprising measuring the prevalence and/or proportional abundance of one or more bacteria in the subject's gut microbiome.

11. The method of claim 10, wherein measuring the prevalence and/or proportional abundance of one or more bacteria in the subject's gut microbiome comprises obtaining one or more times a biological sample from the subject.

12. The method of claim 11, wherein a first obtaining step is performed prior to administering any treatment to the subject.

13. The method of claim 12, wherein a second obtaining step is performed after administering any treatment to the subject.

14. The method of claim 11, further comprising comparing the prevalence and/or proportional abundance of the one or more bacteria in the subject's gut microbiome to one or more standard levels, and if the prevalence and/or proportional abundance of the one or more bacteria in the subject's gut microbiome is similar to that of one or more subjects having demonstrated responsiveness to treatment, then characterizing the subject as likely to be responsive to treatment.

15. The method of claim 11, further comprising comparing the prevalence and/or proportional abundance of the one or more bacteria in the subject's gut microbiome to one or more standard levels, and if the prevalence and/or proportional abundance of the one or more bacteria in the subject's gut microbiome is dissimilar to that of one or more subjects having demonstrated responsiveness to treatment, then characterizing the subject as likely to be non-responsive to treatment.

16. The method of claim 1, wherein, following the administering step, (i) the proportional abundance of Asaccharobacter, Bifidobacterium, Collinsella, Eggerthella, Gardnerella, Gordonibacter, or any combination thereof in the gut microbiome of the subject is decreased, and/or (ii) the proportional abundance of Alistipes and/or Akkermansia in the gut microbiome of the subject is increased.

17. The method of claim 5, wherein, following the administering step, the proportional abundance of one or more E. coli sequence type 131 (ST131) bacteria in the gut microbiome of the subject is decreased.

18. The method of claim 1, further comprising treating the subject by administering to the subject a composition comprising (i) one or more agents that reduce the intestinal carriage associated with the colonization of drug-resistant E. coli bacteria, (ii) one or more agents that reduce the proportional abundance of one or more bacteria associated with the carriage and/or persistence of drug-resistant E. coli bacteria, (iii) one or more agents that inhibit proliferation of Asaccharobacter, Bifidobacterium, Collinsella, Eggerthella, Gardnerella, Gordonibacter, or any combination thereof in the gut microbiome of the subject, (iv) one or more agents that increase proliferation of Alistipes and/or Akkermansia, or (v) any combination thereof.

19. The method of claim 18, further comprising repeating the treating step one or more times until the subject experiences an alleviation and/or a diminishment of one or more symptoms.

20. A method of reducing the risk of developing a drug-resistant E. coli infection, the method comprising:
administering to a subject at risk of developing a drug-resistant E. coli infection a therapeutically effective amount of a composition,
wherein, following the administering step, the composition reduces the intestinal carriage associated with the colonization of drug-resistant E. coli bacteria, thereby reducing the risk of an infection.
